SEQUENCE LISTING (19 sequences)

SEQ ID NO: 1 - DNA, 2220 bp, human
ccgcacaactgtaaccgctgccccggccgccgcccgctccttctcgggccggcgggcacagagcgcagcgcggcggggccggcggcatggctgtgtcctggaggagctggctcgccaacgaaggggttaaacacctctgcctgttcatctggctctccatgaatgtcctgcttttctggaaaaccttcttgctgtataaccaagggccagagtatcactacctccaccagatgttggggctaggattgtgtctaagcagagcctcagcatctgttcttaacctcaactgcagccttatccttttacccatgtgccgaacactcttggcttacctccgaggatcacagaaggttccaagcaggagaaccaggagattgttggataaaagcagaacattccatattacctgtggtgttactatctgtattttctcaggcgtgcatgtggctgcccatctggtgaatgccctcaacttctcagtgaattacagtgaagactttgttgaactgaatgcagcaagataccgagatgaggatcctagaaaacttctcttcacaactgttcctggcctgacaggggtctgcatggtggtggtgctattcctcatgatcacagcctctacatatgcaataagagtttctaactatgatatcttctggtatactcataacctcttctttgtcttctacatgctgctgacgttgcatgtttcaggagggctgctgaagtatcaaactaatttagatacccaccctcccggctgcatcagtcttaaccgaaccagctctcagaatatttccttaccagagtatttctcagaacattttcatgaacctttccctgaaggattttcaaaaccggcagagtttacccagcacaaatttgtgaagatttgtatggaagagcccagattccaagctaattttccacagacttggctttggatttctggacctttgtgcctgtactgtgccgaaagactttacaggtatatccggagcaataagccagtcaccatcatttcggtcataagtcatccctcagatgtcatggaaatccgaatggtcaaagaaaattttaaagcaagacctggtcagtatattactctacattgtcccagtgtatctgcattagaaaatcatccatttaccctcacaatgtgtccaactgaaaccaaagcaacatttggggttcatcttaaaatagtaggagactggacagaacgatttcgagatttactactgcctccatctagtcaagactccgaaattctgcccttcattcaatctagaaattatcccaagctgtatattgatggtccttttggaagtccatttgaggaatcactgaactatgaggtcagcctctgcgtggctggaggcattggagtaactccatttgcatcaatactcaacaccctgttggatgactggaaaccatacaagcttagaagactatactttatttgggtatgcagagatatccagtccttccgttggtttgcagatttactctgtatgttgcataacaagttttggcaagagaacagacctgactatgtcaacatccagctgtacctcagtcaaacagatgggatacagaagataattggagaaaaatatcatgcactgaattcaagactgtttataggacgtcctcggtggaaacttttgtttgatgaaatagcaaaatataacagaggaaaaacagttggtgttttctgttgtggacccaattcactatccaagactcttcataaactgagtaaccagaacaactcatatgggacaagatttgaatacaataaagagtctttcagctgaaaacttttgccatgaagcaggactctaaagaaggaatgagtgcaatttctaagactttgaaactcagcggaatcaatcagctgtgttatgccaaagaatagtaaggttttcttatttatgattatttgaaaatggaaatgtgagaatgtggcaacatgaccgtcacattacatgtttaatctggaaaccaaagagaccctgaagaatatttgatgtgatgattcattttcagttctcaaattaaaagaaaactgttagatgcacactgttgattttcatggtggattcaagaactccctagtgaggagctgaacttgctcaatctaaggctgattgtcgtgttcctctttaaattgtttttggttgaacaaatgcaagattgaacaaaattaaaaat

SEQ ID NO: 2 - protein, 578 aa, human
MAVSWRSWLANEGVKHLCLFIWLSMNVLLFWKTFLLYNQGPEYHYLHQMLGLGLCLSRASASVLNLNCSLILLPMCRTLLAYLRGSQKVPSRRTRRLLDKSRTFHITCGVTICIFSGVHVAAHLVNALNFSVNYSEDFVELNAARYRDEDPRKLLFTTVPGLTGVCMVVVLFLMITASTYAIRVSNYDIFWYTHNLFFVFYMLLTLHVSGGLLKYQTNLDTHPPGCISLNRTSSQNISLPEYFSEHFHEPFPEGFSKPAEFTQHKFVKICMEEPRFQANFPQTWLWISGPLCLYCAERLYRYIRSNKPVTIISVISHPSDVMEIRMVKENFKARPGQYITLHCPSVSALENHPFTLTMCPTETKATFGVHLKIVGDWTERFRDLLLPPSSQDSEILPFIQSRNYPKLYIDGPFGSPFEESLNYEVSLCVAGGIGVTPFASILNTLLDDWKPYKLRRLYFIWVCRDIQSFRWFADLLCMLHNKFWQENRPDYVNIQLYLSQTDGIQKIIGEKYHALNSRLFIGRPRWKLLFDEIAKYNRGKTVGVFCCGPNSLSKTLHKLSNQNNSYGTRFEYNKESFS

SEQ ID NO: 3 - DNA, 1893 bp, mouse
ttcggtcctgagcaccgagcagggcgcagcactccccgcgccggcggcatggcggtgtcctggaggagctggctggccaacgaaggggttaaacacctctgcctgctcatttggctgtccctaaacgttctacttttctggaaaaccttcctgctgtacaaccaagggccagaatactactacattcaccaaatgttgggcctaggattgtgtttaagcagagcatctgcatctgtcctgaacctcaactgcagcctcatccttttacctatgtgccggacagtcctggcttatcttcgaggatcacagaaggtccctagcaggagaacaagaagattgttggataaaagcaagactctacacatcacatgtggtgtaactatctgtattttctcaggtgtgcatgtagccgcccacttggtgaatgccctcaacttttcagtgaactacagtgaagatttccttgaactgaatgcagcaagataccagaatgaggatcccagaaagcttctcttcacaaccattcctggtctgacgggtgtctgcatggtggtggtattgttcctcatggttacagcttctacctacgcaataagagtttctaattatgatatcttctggtatactcacaacctcttctttgtcttctacatgctgctgctgttgcatgtttcaggtggtttgttgaagtatcagacaaatgtagacactcaccctcctggctgcattagtcttaaccagacatcatcccagaatatgtccataccagactacgtctcagaacattttcatggatctttgcctcgagggttttcaaaattagaagatcgttaccagaaaacacttgtgaagatttgcctggaagaacccaagttccaagctcatttcccacagacctggatttggatttctggacctttgtgcctttattgtgcggagagactttaccgatgcatcaggagcaacaaacctgtcaccatcatctcagtcatcaatcatccctctgatgtaatggaactccgtatgatcaaagaaaactttaaagcaagacctggtcagtatattattctccattgccccagtgtatcagcattagaaaaccacccatttactctcacaatgtgtcctactgaaaccaaagcaacatttggtgtccactttaaagtagtaggagactggacagaacgattccgggatttgctactgcctccatcaagtcaagactctgagattctacccttcattcactctagaaattaccctaagttatacattgatggtccatttggaagcccatttgaggagtcactgaactatgaagttagtctctgtgtggctggaggcattggagtcactccatttgcatcgatactaaacactctactggatgactggaaaccatacaagttaagaagactgtactttatctgggtgtgcagagacatccaatcattccagtggtttgcagatttactctgtgtgttgcataacaagttttggcaagaaaacagacctgactttgtgaacatccagctgtacctcagtcaaacagatgggattcagaagataattggagaaaaatatcacacactgaattcgagacttttcattgggcgtcctcggtggaaacttttatttgatgaaatagcaaaatgtaacagagggaaaacagttggagttttctgctgtggacccagttctatttccaagactcttcatagtttgagtaaccggaacaactcatatgggacaaaatttgaatacaataaagaatccttcagctgaaaccttaggagactactggggactttaaagaggaacaagtgcaatttctaagacttagagactcagctgaatcaaacagctgtgctatgccaaagaataccaagggtt

SEQ ID NO: 4 - protein, 578 aa, mouse
MAVSWRSWLANEGVKHLCLLIWLSLNVLLFWKTFLLYNQGPEYYYIHQMLGLGLCLSRASASVLNLNCSLILLPMCRTVLAYLRGSQKVPSRRTRRLLDKSKTLHITCGVTICIFSGVHVAAHLVNALNFSVNYSEDFLELNAARYQNEDPRKLLFTTIPGLTGVCMVVVLFLMVTASTYAIRVSNYDIFWYTHNLFFVFYMLLLLHVSGGLLKYQTNVDTHPPGCISLNQTSSQNMSIPDYVSEHFHGSLPRGFSKLEDRYQKTLVKICLEEPKFQAHFPQTWIWISGPLCLYCAERLYRCIRSNKPVTIISVINHPSDVMELRMIKENFKARPGQYIILHCPSVSALENHPFTLTMCPTETKATFGVHFKVVGDWTERFRDLLLPPSSQDSEILPFIHSRNYPKLYIDGPFGSPFEESLNYEVSLCVAGGIGVTPFASILNTLLDDWKPYKLRRLYFIWVCRDIQSFQWFADLLCVLHNKFWQENRPDFVNIQLYLSQTDGIQKIIGEKYHTLNSRLFIGRPRWKLLFDEIAKCNRGKTVGVFCCGPSSISKTLHSLSNRNNSYGTKFEYNKESFS

SEQ ID NO: 5 - DNA, 462 bp, human
gtcagcctctgcgtggctggaggcattggagtaactccatttgcatcaatactcaacaccctgttggatgactggaaaccatacaagcttagaagactatactttatttgggtatgcagagatatccagtccttccgttggtttgcagatttactctgtatgttgcataacaagttttggcaagagaacagacctgactatgtcaacatccagctgtacctcagtcaaacagatgggatacagaagataattggagaaaaatatcatgcactgaattcaagactgtttataggacgtcctcggtggaaacttttgtttgatgaaatagcaaaatataacagaggaaaaacagttggtgttttctgttgtggacccaattcactatccaagactcttcataaactgagtaaccagaacaactcatatgggacaagatttgaatacaataaagagtctttcagc

SEQ ID NO: 6 - protein, 154 aa, human
VSLCVAGGIGVTPFASILNTLLDDWKPYKLRRLYFIWVCRDIQSFRWFADLLCMLHNKFWQENRPDYVNIQLYLSQTDGIQKIIGEKYHALNSRLFIGRPRWKLLFDEIAKYNRGKTVGVFCCGPNSLSKTLHKLSNQNNSYGTRFEYNKESFS

SEQ ID NO: 7 - DNA, 462 bp, mouse
gttagtctctgtgtggctggaggcattggagtcactccatttgcatcgatactaaacactctactggatgactggaaaccatacaagttaagaagactgtactttatctgggtgtgcagagacatccaatcattccagtggtttgcagatttactctgtgtgttgcataacaagttttggcaagaaaacagacctgactttgtgaacatccagctgtacctcagtcaaacagatgggattcagaagataattggagaaaaatatcacacactgaattcgagacttttcattgggcgtcctcggtggaaacttttatttgatgaaatagcaaaatgtaacagagggaaaacagttggagttttctgctgtggacccagttctatttccaagactcttcatagtttgagtaaccggaacaactcatatgggacaaaatttgaatacaataaagaatccttcagc

SEQ ID NO: 8 - protein, 154 aa, mouse
VSLCVAGGIGVTPFASILNTLLDDWKPYKLRRLYFIWVCRDIQSFQWFADLLCVLHNKFWQENRPDFVNIQLYLSQTDGIQKIIGEKYHTLNSRLFIGRPRWKLLFDEIAKCNRGKTVGVFCCGPSSISKTLHSLSNRNNSYGTKFEYNKESFS

SEQ ID NO: 9 - DNA, 21 bp, Artificial Sequence
  Synthetic
tgttgggcctaggattgtgtt

SEQ ID NO: 10 - DNA, 29 bp, Artificial Sequence
  Synthetic
aagcagagcatctgcatctgtcctcaacc

SEQ ID NO: 11 - DNA, 21 bp, Artificial Sequence
  Synthetic
aaaaggatgaggctgcagttg

SEQ ID NO: 12 - DNA, 23 bp, Artificial Sequence
  Synthetic
tggtcatgcagcattaaactttg

SEQ ID NO: 13 - DNA, 21 bp, Artificial Sequence
  Synthetic
cattgtcccacattggtctcc

SEQ ID NO: 14 - DNA, 20 bp, Artificial Sequence
  Synthetic
cggctaccacatccaaggaa

SEQ ID NO: 15 - DNA, 22 bp, Artificial Sequence
  Synthetic
tgctggcaccagacttgccctc

SEQ ID NO: 16 - DNA, 18 bp, Artificial Sequence
  Synthetic
gctggaattaccgcggct

SEQ ID NO: 17 - DNA, 20 bp, Artificial Sequence
  Synthetic
ttggccagccagctcctcca

SEQ ID NO: 18 - DNA, 20 bp, Artificial Sequence
  Synthetic
taggccagcaagctcctaca

SEQ ID NO: 19 - DNA, 20 bp, Artificial Sequence
  Synthetic
cgtcacgctcagctcaccgt

(19) United States
(12) Patent Application Publication
(10) Pub. No.: US 2007/0037883 A1
(43) Pub. Date: Feb. 15, 2007

(54) Therapeutic compositions

(76) Inventors: Gregory James Dusting, KEW, VICTORIA (AU); Grant Raymond Drummond, Healesville, Victoria (AU); Christopher Graeme Sobey, Lara, Victoria (AU)

Correspondence Address: JENNIFER M MCCALLUM, PH D, ESQ;THE MCCALLUM LAW FIRM, LLC, 685 BRIGGS STREET, PO BOX 929, ERIE, CO, 80516, US

(21) Appl. No.: 10/547,613

(22) PCT Filed: Sep. 14, 2004
(86) PCT No.: PCT/AU04/00254

§ 371 Date: Jan. 3, 2006

Related U.S. Application Data
(60) Provisional application No. 60/450,892, filed on Feb. 28, 2003.

(51) Int. Cl. A61K 31/185

(52) U.S. Cl. 514/553

(57) ABSTRACT

The present invention provides compounds, compositions and methods for inhibiting or reducing reactive oxygen species (ROS) production in cells, such as in cells of the vascular system and in particular the smooth muscle-containing vasculature and/or endothelial cell-containing vasculature and/or adventitial fibroblast-containing vasculature. ROS production may also be inhibited in non-vascular cells of animals including mammals such as humans. Non-vascular cells contemplated herein include nerve cells, stem cells, progenitor cells and some cancer and rumor cells. More particularly, the present invention provides agents and even more particularly, cell-impermeable agents, capable of modulating NADPH oxidase activity, function or levels, thereby controlling superoxide production and production of downstream ROS. The present invention particularly enables agents which are selective against a form of Nox4-containing NADPH oxidase which has a portion of the enzyme such as all or part of the Nox4 component extracellularly exposed.

### BACKGROUND OF THE INVENTION

1. Field of the Invention

The present invention provides compounds, compositions and methods for inhibiting or reducing reactive oxygen species' (ROS) production in cells, such as in cells of the vascular system and in particular the smooth muscle-containing vasculature and/or endothelial cell-containing vasculature and/or adventitial fibroblast-containing vasculature and/or non-vascular systems. ROS production may also be inhibited in non-vascular cells of animals including mammals such as humans. Non-vascular cells contemplated herein include nerve cells, stem cells, progenitor cells and some cancer and tumor cells. More particularly, the present invention provides agents and even more particularly, cell-impermeable agents, capable of modulating NADPH oxidase activity, function or levels, thereby controlling superoxide production and production of downstream ROS. The present invention particularly enables agents which are selective against a form of Nox4-containing NADPH oxidase which has a portion of the enzyme such as all or part of the Nox4 component extracellularly exposed.

2. Description of the Prior Art

Bibliographic details of the publications referred to by author in this specification are collected at the end of the description.

Reference to any prior art in this specification is not, and should not be taken as, an acknowledgment or any form of suggestion that this prior art forms part of the common general knowledge in any country.

Reactive oxygen species (ROS) such as hypochlorite, lipid peroxides, peroxynitrite, hydrogen peroxide and hydroxyl radicals as well as the parent species, superoxide, are strongly implicated in the pathogenesis of atherosclerosis, cell proliferation, hypertension and reperfusion injury. Not only is superoxide production, for example, in the arterial wall increased by all risk factors for atherosclerosis, but ROS also induce many “proatherogenic” cellular responses in vitro. These include inactivating endothelium-derived nitric oxide (NO) [Gryglewski et al., Nature 320: 454-456, 1986; Paravicini et al., Circulation Research 91: 54-61, 2002; Dusting et al., Clinical and Experimental Pharmacology and Physiology 25: S34-41, 1998], up-regulating adhesion molecule expression [Lo et al., Am. J. Physiol. 264: L406-412, 1993], stimulating the proliferation and migration of vascular smooth muscle cells (VSMCs) [Griendling and Ushio-Fukai, J. Lab. Clin. Med. 132: 9-15, 1998] and oxidatively modifying lipoproteins [Lynch and Frei, J. Lipid Res. 34: 1745-1753, 1993]. These observations have led to the “oxidative stress hypothesis” of atherogenesis and have sparked interest in the potential benefits of antioxidants as treatments for atherosclerosis. While there is epidemiological evidence of a reduced risk of cardiovascular disease in individuals with a high dietary intake of antioxidants such as vitamins E and C, randomized trials have failed to demonstrate any clinical benefit of antioxidant therapy in individuals at high risk of cardiovascular events [Yusuf et al., N. Engl. J. Med. 342: 154-160, 2000]. There are many reasons why conventional antioxidants may be ineffective against vascular disease. These include poor bioavailability of antioxidants at the site of disease due to insufficient absorption or compartmentalization in aqueous versus lipid phases, as well as slow reaction kinetics of ROS with antioxidants compared to their rapid reaction with important biomolecules such as NO and lipoproteins. In addition, conventional antioxidants act by causing the one electron reduction of superoxide. This results in formation of H₂O₂, which is a proatherogenic molecule in its own right and precursor to even more damaging ROS such as HOCl⁻ and OH^{●}. Clearly, there is a need to devise strategies that remove superoxide rapidly without causing generation of downstream ROS.

The major source of ROS in blood vessels is a superoxide-producing NADPH oxidase [Griendling et al., Cir. Res. 86: 494-501, 2000], similar to the enzyme responsible for the respiratory burst of phagocytes [Babior, Blood 93: 1464-1476, 1999]. NADPH oxidases are made up of a membrane-bound cytochrome b558 domain and three cytosolic protein subunits, p47phox, p67phox and a small G-protein, Rac. The cytochrome domain is a heterodimeric protein comprising a 22 KDa α-subunit, as well as a larger, flavin-containing β-subunit that is required for substrate binding and electron transfer from NADPH to molecular oxygen. When activated, the cytosolic components translocate to the membrane components to allow assembly of the active oxidase enzyme. NADPH oxidase is turned on with intimal hyperplasia induced by periarterial collars [Paravicini et al., 2002, supra; Dusting et al., 1998, supra], genetic hypercholesterolemia [Drummond et al., Circulation 104: II-71, 2001], arterial balloon injury [Shi et al., Arterioscler Thromb. Vasc. Biol. 21: 739-745, 2001], vein grafting [West et al., Arterioscler Thromb. Vasc. Biol. 21: 189-194, 2001] and hypertension [Beswick et al., Hypertension 38: 1107-1111, 2001]. Increased vascular superoxide generation by NADPH oxidase has also been linked to clinical risk factors for atherosclerosis in humans and to impaired endothelial NO function in patients with coronary artery disease [Guzik et al., Cir. Res. 86: E85-90, 2000]. Importantly, targeted disruption of the p47phox subunit of NADPH oxidase in mice clearly reduces superoxide generation in VSMCs and retards significantly hypercholesterolemia-induced atherosclerosis in these animals [Barry-Lane et al., J. Clin. Invest. 108: 1513-1522, 2001]. Collectively, these data provide strong evidence that increased NADPH oxidase activity is not just a symptom of atherosclerosis but a major causative factor in the pathogenesis of the disease.

Recent studies suggest that the nature of the NADPH-binding β-subunit of NADPH oxidase varies depending on cell type. Thus, while gp91phox is associated with p22phox in neutrophils [Babior, 1999, supra], homologs of this protein may be important for NADPH oxidase activity in VSMCs and endothelial cells (Ago et al., Circulation 109(2):227-233, 2004). The first clue that VSMCs express an isoform of NADPH oxidase that is distinct from that of phagocytes was the observation that VSMCs lack gp91phox [Ushio-Fukai et al., J. Biol. Chem. 271: 23317-23321, 1996]. Subsequently, it was shown that VSMCs and whole aortas from gp91phox-null mice were still able to generate superoxide in response to NADPH (substrate for NADPH oxidase) [Barry-Lane et al., 2001, supra; Souza et al., Am. J. Physiol. Heart Circ. Physiol. 280: H658-667, 2001]. Given that gp91phox acts as the critical catalytic subunit of the neutrophil oxidase [Babior, 1999, supra], an alternative catalytic subunit must be involved.

Recently, two novel homologs of gp91phox, termed Nox1 and Nox4, were identified in cultured rat VSMCs [Lassegue et al., Circ. Res. 88: 888-894, 2001]. Nox4 has also been referred to by the name renox. Both of these proteins contain binding sites for NADPH, flavin adenine dinucleotide (FAD) and a heme-moiety, making them strong candidates for the crucial catalytic subunit of vascular NADPH oxidases [Lambeth et al., Trends Biochem. Sci. 25: 459-461, 2000]. Although Nox4 is expressed in VSMCs and whole blood vessels from rabbits and mice, Nox1 expression has not been detected in any of these preparations [Paravicini et al., 2002, supra; Dusting et al., 1998, supra]. Likewise, studies by other groups on freshly isolated human and rat arteries demonstrated a very low Nox1:Nox4 ratio (i.e. <0.5%) [Ritchie et al., European Journal of Pharmaology 461: 171-179, 2003], suggesting that Nox4 is likely to have a greater role in vascular superoxide production than Nox1.

There is a need to identify compounds which can specifically target NADPH oxidase in particular cells such as cells of the vascular and non-vascular systems to thereby reduce direct and downstream generation of ROS. Such compounds are useful in treating a variety of events and conditions including pathologies such as atherosclerosis and arteriosclerosis, cadiovascular complications of Type I and II diabetes, intimal hyperplasia, coronary heart disease, cerebral, coronary or arterial vasospasm, endothelial dysfunction, heart failure including congetive heart failure, sepsis, peripheral artery disease, restenosis and restenosis after angioplasty, stroke, vascular complications after organ transplantation, cardiovascular complications arising from viral and bacterial infections as well as any conditions which may be independent or secondary to another condition including mycardial infarction, hypertension, formation of atherosclerotic plaques, platelet aggregations, angina, aneurysm, transient ischemic attack, abnormal oxygen flow and/or delivery, atrophy or organ damage, pulmonary embolus, thrombotic or a generalized arterial or venous condition including endothelial dysfunction, a thrombotic event including deep vein thrombosis or damage to vessels of the circulatory system or stent failure or trauma caused by a stent, pacemaker or other prosthetic device as well as reperfusion injury including any injury caused after ischemia by restoration of blood flow and oxygen delivery, gangrene, (cancer and/or abnormal tumor), stem or progenitor cell proliferation, respiratory disease (eg. asthma, bronchitis, allergic rhinits and adult respiratory distress syndrome), skin disease (psoriasis, eczema and dermatitis), and various disorders of bone metabolisms (oestoporosis, hyperparathyroidism, oestosclorosis, oestoporasis and periodontits) and renal failure.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of the sequence identifiers is provided in Table 1. A sequence listing is provided after the claims.

The present invention is predicated in part on the identification of an extracellularly exposed component of NADPH oxidase such as all or part of Nox4 (also known as renox) which is a critical catalytic subunit of NADPH oxidase expressed by a variety of cells within the vascular and non-vascular systems. The vascular system includes smooth muscle-containing vasculature and/or endothelial cell-containing vasculature and/or adventitial fibroblast-containing vasculature. Non-vascular systems include nerve cells, cancer cells, fibroblasts and stem and progenitor cells. The form of NADPH oxidase of interest is the form comprising Nox4 which is distinguishable from the gp91phox-containing NADPH oxidase isoform present in leukocytes and phagocytes due to the extracellular expression of all or a portion of Nox4 which contains the NADPH binding doman. The leukocytes and phagocytic isoforms of NADPH oxidase comprise a Nox4 homolog, i.e. gp91phox. The present invention provides, therefore, compounds which selectively inhibit NADPH oxidases which contain an extracellularly exposed Nox4 NADPH binding site. Particularly useful compounds include cell impermeable Nox4 antagonists or inhibitors. The ability to selectively inhibit the Nox4-containing forms of NADPH oxidase enables inhibition of superoxide generation and downstream reactive oxygen species (ROS) formation such as hypochlorite, lipid peroxides, peroxynitrite, hydrogen peroxide and hydroxyl radicals from cells such as vascular smooth muscle cells (VSMC) endothelial-cells and/or adventitial fibroblast vasculature which is proposed to be responsible at least in part for the development of pathologies such as atherosclerosis and arteriosclerosis, cadiovascular complications of Type I and II diabetes, intimal hyperplasia, coronary heart disease, cerebral, coronary or arterial vasospasm, endothelial dysfunction, heart failure including congetive heart failure, sepsis, peripheral artery disease, restenosis and restenosis after angioplasty, stroke, vascular complications after organ transplantation, cardiovascular complications arising from viral and bacterial infections as well as any conditions which may be independent or secondary to another condition including mycardial infarction, hypertension, formation of atherosclerotic plaques, platelet aggregations, angina, aneurysm, transient ischemic attack, abnormal oxygen flow and/or delivery, atrophy or organ damage, pulmonary embolus, thrombotic or a generalized arterial or venous condition including endothelial dysfunction, a thrombotic event including deep vein thrombosis or damage to vessels of the circulatory system or stent failure or trauma caused by a stent, pacemaker or other prosthetic device as well as reperfusion injury including any injury caused after ischemia by restoration of blood flow and oxygen delivery, gangrene, (cancer and/or abnormal tumor), stem or progenitor cell proliferation, respiratory disease (eg. asthma, bronchitis, allergic rhinits and adult respiratory distress syndrome), skin disease (psoriasis, eczema and dermatitis), and various disorders of bone metabolisms (oestoporosis, hyperparathyroidism, oestosclorosis, oestoporasis and periodontits) and renal failure.

The present invention provides, therefore, compounds which inhibit an NADPH oxidase comprising an extracellularly exposed Nox4 in particular cells such as VSMC— and/or endothelial-containing vasculature and/or adventitial fibroblast-containing vasculature and fibroblasts, stem cells, nerve cells and cancer cells. The compounds of the present invention include small or large chemical molecules, peptides, polypeptides and proteins, antibodies (including polyclonal, monoclonal, deimmunized chimeric recombinant and synthetic immunointeractive molecule) and nucleic acid molecules or their analogs including antisense oligonucleotides, sense oligonucleotides or full length sense nucleic acid molecules useful in inducing co-suppression or other RNAi-mediated gene silencing events. Reference to “RNAi” includes “siRNA”. The nucleic acid molecules may also be modified such as comprising a C5 propynl modification or a full phosphorothioate modification. Particularly useful compounds are cell impermeable Nox4 inhibitors and/or antagonists.

Particularly useful compounds contemplated by the present invention are benzamide and aryl sulphonates and derivatives or analogs such as suramin or its derivatives, analogs or homologs. Other useful compounds include Reactive blue-2 [1-amino-4[[4[[4-chloro-6-[[n-sulfophenyl]amino]-1,3,5-triazin-2-yl]amino]-3-sulfophenyl]amino]-9,10-dihydro-9,10-dioxo-2-anthracene-sulfonic acid wherein n is 3 or 4] and PPADS [pyridoxal phosphate-6-axo(benzene-2,4-disulfonic acid)] or 4-[[-formyl-5-hydroxy-6-methyl-3-[(phos-phonooxy)methyl]-2-pyridinyl]azo]-1,3-benzenedisulfonic acid. Tempol (4-hydroxy-2,2,6,6-tetramethyl piperidinoxyl) and DPI (diphenyleneiodonium) or derivatives, analogs or homologs thereof as well as a range of genetic agents for use in gene therapy such as nucleotide anti-sense and sense molecules are also contemplated for use in accordance with the present invention. Other useful compounds include agonists of Nox4-inhibitor interaction. An “agonist” includes a compound which potentiates the inhibitory activity of Nox4 antagonists such as suramin.

The present invention provides pharmaceutical compositions comprising the compounds of the present invention and contemplates methods of treating or preventing or otherwise ameliorating the symptoms of or associated with pathologies such as atherosclerosis and arteriosclerosis, cardiovascular complications of Type I and II diabetes, intimal hyperplasia, coronary heart disease, cerebral, coronary or arterial vasospasm, endothelial dysfunction, heart failure including congestive heart failure, sepsis, peripheral artery disease, restenosis and restenosis after angioplasty, stroke, vascular complications after organ transplantation, cardiovascular complications arising from viral and bacterial infections as well as any conditions which may be independent or secondary to another condition including myocardial infarction, hypertension, formation of atherosclerotic plaques, platelet aggregations, angina, aneurysm, transient ischemic attack, abnormal oxygen flow and/or delivery, atrophy or organ damage, pulmonary embolus, thrombotic or a generalized arterial or venous condition including endothelial dysfunction, a thrombotic event including deep vein thrombosis or damage to vessels of the circulatory system or stent failure or trauma caused by a stent, pacemaker or other prosthetic device as well as reperfusion injury including any injury caused after ischemia by restoration of blood flow and oxygen delivery, gangrene, (cancer and/or abnormal tumor), stem or progenitor cell proliferation, respiratory disease (eg. asthma, bronchitis, allergic rhinitis and adult respiratory distress syndrome), skin disease (psoriasis, eczema and dermatitis), and various disorder of bone metabolisms (oestoporosis, hyperparathyroidism, oestosclorosis, oestoporasis and periodontits) and renal failure.

A summary of sequence identifiers used throughout the subject specification is provided in Table 1.

TABLE 1
Summary of sequence identifiers

| Sequence ID NO: | Description |
|---|---|
| 1 | mRNA sequence encoding human Nox4 |
| 2 | Amino acid sequence of human Nox4 |
| 3 | mRNA sequence encoding mouse Nox4 |
| 4 | Amino acid sequence of mouse Nox4 |
| 5 | mRNA sequence encoding extracellular C-terminal |
| | portion of human Nox4 |
| 6 | Amino acid sequence of extracellular C-terminal |
| | portion of mouse human Nox4 |
| 7 | mRNA sequence encoding extracellular C-terminal |
| | portion of mouse Nox4 |
| 8 | Amino acid sequence of extracellular C-terminal |
| | portion of mouse mouse Nox4 |
| 9 | primer |
| 10 | probe |
| 11-14 | primer |
| 15 | probe |
| 16 | primer |
| 17-19 | oligonucleotide |

A list of abbreviations used herein is provided in Table 2.

TABLE 2
Abbreviations

| Abbreviation | Ddescription |
|---|---|
| ROS | reactive oxygen species¹ |
| VSMC | vascular smooth muscle cell |
| NO | nitric oxide |
| FAD | flavin adenine dinucleotide |
| tempol | 4-hydroxy-2,2,6,6-tetramethyl piperidinoxyl |
| DPI | diphenyleneiodonium |
| ICAM-1 | intercellular adhesion molecule-1 |
| VCAM-1 | vascular cell adhesion molecule-1 |
| MCP-1 | monocyte chemoattractant protein-1 |
| IEL | internal elastic lamina |
| BrdU | bromodeoxyuridine |
| PCR | polymerase chain reaction |
| DHE | dihydroethidium |
| DCFH-DA | 2′-7′dichlorofluorescein diacetate |
| PPADS | [pyridoxal phosphate-6-axo(benzene-2,4-disulfonic acid)] |

¹includes superoxide, hydrogen peroxide, hydroxyl radicals, peroxynitrite, hypochlorite, lipid peroxides, etc.

### BRIEF DESCRIPTION OF THE FIGURES

FIG. 1 is a diagrammatic and tabular representation of the chemical structure of suramin and the structural features of suramin analogs [Jentsch et al., J. Gen. Virol. 68: 2183-2192, 1987; U.S. Pat. No. 5,173,509].

FIG. 2 is a graphical representation showing the effect of suramin as a selective inhibitor of Nox4-containing vascular NADPH-oxidase. Suramin (≦100 μM fully inhibits 100 μM NADPH-driven activity of Nox4-containing NADPH-oxidase in mouse vascular smooth muscle cells (VSMC, a) and endothelium, but has little effect on gp91phox-containing NADPH-oxidase in the mouse macrophage cell line J774 (b) (*P<0.05 vs Control).

FIG. 3 is a representation of the mRNA and corresponding amino acid sequence of human Nox4. The highlighted regions encode or comprise the amino acid constituting the putative suramin binding site [http://www.biochem.emory.edu/labs/dlambe/noxfamilypage.html#noxfamily]; the underlined sequences encode or comprise the predicted extracellular C-terminal domain

FIG. 4 is a representation of the mRNA and corresponding amino acid sequence of mouse Nox4. The highlighted regions encode or comprise the amino acid constituting the putative suramin binding site [http://www.biochem.emory.edu/labs/dlambe/noxfamilypage.html#noxfamily]; the underlined sequences encode or comprise the predicted extracellular C-terminal domain.

FIGS. 5A-C are graphical representations showing characterization of NADPH-dependent superoxide production in cultured mouse VSMCs. In (A), superoxide production was measured after 45 mins of incubation with increasing concentrations of NADPH. In (B), VSMCs were incubated for 45 mins with 100 μM NADPH, either alone or in the presence of vehicle (DMSO 0.1%) or increasing concentrations of DPI. In (C), VSMCs were incubated for 24 h in DMEM containing 5% v/v FBS and either vehicle (DMSO 0.1%) or increasing concentrations of apocynin. Cells were then treated for 45 mins with 100 μM NADPH in the absence or presence of vehicle or apocynin. In all experiments, superoxide was measured by 5 μmol/L lucigenin-enhanced chemiluminescence. Values (mean±SEM from four to eight experiments) are expressed either as counts per second per viable cell (A) or as a percentage of the counts per second per viable cell obtained in cells treated with NADPH alone (B & C). *P<0.05 vs vehicle.

FIGS. 6A and 6B are graphical representations showing Nox4 mRNA expression in cultured mouse VSMCs. Total RNA, extracted from cultured mouse VSMCs or from freshly isolated mouse VSMCs and whole aortas, was reverse transcribed, and 5 ng of cDNA was then used in real-time PCR to examine expression of Nox4. In (A), upper panel is a representative trace of FAM (Nox4)-dependent fluorescence intensity versus PCR cycle number measured in a single VSMC culture (note reaction performed in triplicate). The lower panel shows VIC (18S)-dependent fluorescence versus PCR cycle number in the same reactions. As a control for genomic DNA contamination, real-time PCR was also performed using, as a template, the same RNA sample that had not been reverse transcribed (-RT). (B) Grouped data showing Nox4 expression relative to a “reference” sample in cultured VSMCs versus freshly isolated VSMCs and whole aortas. Values are mean±SEM from four to eight experiments. 5 ng of the cDNA was used for each real-time PCR reaction to examine the expression of Nox4 relative to 18S rRNA.

FIG. 7 is a graphical representation showing optimization experiment showing time- and concentration-dependent effects of Nox4 antisense on NADPH-driven superoxide production in cultured mouse VSMCs. VSMCs were incubated for up to 72 h with increasing concentrations of Nox4 antisense (sequence +13/+33). Cells were then incubated for 45 mins with NADPH (100 μmol/L) and superoxide was assayed using 5 μmol/L lucigenin-enhanced chemiluminescence. Values (mean±SEM from four experiments) are counts per second per viable cell number normalized as a percentage of the same values obtained in cells treated with the transfection reagent alone.

FIG. 8 is a graphical representation showing specific antisense effect of the +13/+33 Nox4 antisense sequence on NADPH-driven superoxide production in cultured mouse VSMCs. VSMCs were incubated for 24 h with the transfection reagent alone (8 μL/mL; open bars) or in the presence of 500 nmol/L antisense (closed bars), mismatch (hatched bars) or scrambled oligonucleotides (vertical lines) prior to being treated for 45 mins with NADPH (100 μmol/L). Superoxide production was then assayed using 5 μmol/L lucigenin-enhanced chemiluminescence. Values (mean±SEM from eight separate experiments) are counts per second per viable cell number normalized as a percentage of the same values obtained in untreated cells. *P<0.05, ***P<0.001.

FIG. 9 is a graphical representation showing specific antisense effect of the +13/+33 Nox4 antisense sequence on Nox4 mRNA expression in cultured mouse VSMCs. VSMCs were incubated for 24 h with the transfection reagent alone (8 μL/mL; open bars) or in the presence of 500 nmol/L antisense (closed bars), mismatch (hatched bars) or scrambled oligonucleotides (vertical lines). RNA was then extracted and reverse transcribed into cDNA, 5 ng of which was used as a template in subsequent real-time PCR to measure Nox4 expression. Nox4 expression was normalized to the 18S expression in the respective sample (ΔCt). The ΔCt value obtained in each sample was then further normalized by subtracting the ΔCt obtained in a “reference” sample (ΔΔCt) and this value was ultimately used in the equation 2^{ΔΔCt}. Values (mean±SEM) are from seven separate experiments. ***P<0.001.

FIGS. 10A and 10B are graphical representations showing effects of Reactive blue-2 and PPADs on NADPH-stimulated superoxide production in mouse vascular smooth muscle cells. Cells were incubated for 45 mins with 100 μM NADPH, either alone or in the presence of increasing concentrations of Reactive blue-2 (A) or PPADS (B). Superoxide production was then measured by 5 μM lucigenin-enhanced chemiluminescence. Values (mean±SEM from four to six experiments) are expressed as a percentage of the counts/second/viable cells obtained in untreated cells.

FIG. 11 shows PSORT predictions results of transmembrane domains and topology of (A) Nox4 and (B) gp91phox. (C) Schematic diagram showing predicted models of Nox4 and gp91phox. Note the NADPH binding site (white bars) is predominantly extracellular on Nox4 but intracellular on gp91phox. Left hand panels in (A) show membrane topology summaries while right hand panels showed the hydrophobicity plots (membrane domains boxed).

FIG. 12 is a graphical representation showing the effect of suramin on atherosclerotic lesion formation. Chronic administration of suramin (15 mg/kg per week for 4 months, n=5) to ApoE−/− mice from 12 weeks of age, and which were also fed a high-fat diet, results in a smaller lesion area over the whole aorta (a), and specifically in the thoracic and abdominal aortic segments (b) than in vehicle(saline)-treated mice (n=6). There was no effect on lesion size in the aortic arch (b). (*P<0.05 vs Vehicle-treated).

FIG. 13 is a graphical representation showing the effect of suramin on the increase in cerebral artery NADPH-oxidase activity after subarachnoid hemorrhage (SAH). Chronic administration of suramin (30-300 mg/kg over 7 days) has no effect on NADPH (100 μM)-driven superoxide production by the isolated basilar artery from control Sprague-Dawley rats (CON, n=17 vs SUR, n=5). By contrast, in rats subjected to SAH, suramin treatment in vivo prevents the increase in superoxide production observed in vitro 2 days after SAH (SAH, n=9 vs SAH+SUR, n=13). (*P<0.05 vs all other groups).

FIG. 14 is a graphical representation showing the effect of suramin on the impairment of endothelium-dependent dilatation in cerebral arteries in vivo after subarachnoid hemorrhage (SAH). Concentration-dependent increases in diameter of the rat basilar artery in vivo which normally occur in control rats in response to acetylcholine (CON, n=7) are markedly impaired in rats 2 days after SAH (SAH, n=8). By contrast, chronic administration of suramin (300 mg/kg s.c. over 7 days) prevents any significant reduction in the response to acetylcholine in vivo after SAH (SAH+SUR, n=8). (*P<0.05 vs SAH).

FIG. 15 is a graphical representation showing the effect of suramin on angiotensin II-induced hypertension. Chronic administration of suramin (150 mg/kg s.c. per week for 2 weeks) had no effect on the mean arterial blood pressure of control rats measured under ketamine-xylazine anaesthesia (CON, n=4 vs SUR, n=4). Chronic administration of angiotensin II alone (5 mg/kg s.c. over 1 week) caused marked hypertension (Ang II, n=7; *P<0.05 vs CON), whereas chronic treatment with suramin for 1 week prior to, and also during 1 week of angiotensin administration resulted in a smaller increase in arterial pressure (Ang II+SUR, n=8; *P<0.05 vs Ang II).

### DETAILED DESCRIPTION OF THE PREFERRED EMBODIMENTS

The present invention provides compounds which selectively target a sub-component of an NADPH oxidase which is substantially unique to a particular cell type and in particular to vascular cells. The latter cells include cells in the smooth muscle-containing vasculature and/or endothelial cell-containing vasculature and/or adventitial fibroblast-containing vasculature. The present invention is also applicable to non-vascular cells including fibroblasts, nerve cells, cancer cells and stem and progenitor cells. More particularly, the present invention provides compounds which target a sub-component which is extracellularly exposed. Even more particularly the sub-component is all or part of the Nox4 component of NADPH oxidase or a homolog of Nox4 present on particular cells such as but not limited to vascular smooth muscle cells (VSMCs) and/or endothelial cell-containing vasculature and/or adventitial fibroblast-containing vasculature and/or non-vascular systems. The present invention provides, therefore, antagonists which selectively target an NADPH oxidase comprising an extracellularly exposed portion of Nox4. The compounds of the present invention are selective in the sense that they do not substantially affect the homologous gp91phox component when the NADPH binding domain is located intraceullarly such as in the NADPH oxidase in leukocytes and phagocytes. In a preferred embodiment, the inhibitors and/or antagonists of Nox4 are cell impermeable compounds and are unable to target an intracellular compound of NADPH oxidase.

Before describing the present invention in detail, it is to be understood that unless otherwise indicated, the subject invention is not limited to specific formulation components, manufacturing methods, dosage regimens, or the like, as such may vary. It is also to be understood that the terminology used herein is for the purpose of describing particular embodiments only and is not intended to be limiting.

It must be noted that, as used in the subject specification, the singular forms “a”, “an” and “the” include plural aspects unless the context clearly dictates otherwise. Thus, for example, reference to a “solvent” includes a single solvent, as well as two or more solvents; reference to “an active agent” includes a single active agent, as well as two or more active agents; and so forth.

In describing and claiming the present invention, the following terminology will be used in accordance with the definitions set forth below.

The terms “compound”, “active agent”, “pharmacologically active agent”, “medicament”, “active” and “drug” are used interchangeably herein to refer to a chemical compound that induces a desired pharmacological, physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc. The term “compound” is not to be construed as a chemical compound only but extends to peptides, polypeptides and proteins and antibodies as well as genetic molecules such as RNA, DNA and chemical analogs thereof.

The present invention contemplates, therefore, compounds useful in preventing or ameliorating physiological and pathophysiological events or symptoms within the vasculature and non-vasculature systems. The term “vasculature” includes smooth muscle cell-containing vasculature and/or endothelial cell-containing vasculature and/or adventitial fibroblast-containing vasculature. Non-vascular systems contemlaced herein include fibroblasts, nerve cells, cancer cells, progenten and stem cells.

A condition or event associated with the vasculature includes a condition characterized or including pathological changes to any or all compartments or anatomical divisions of the cardiovascular system which includes the systemic vasculature of one or more organs. A condition or event associated with the VSMC- and/or endothelial cell- and/or adventitial fibroblast-containing vasculature and/or endothelial cell-containing vasculature and/or adventitial fibroblast-containing vasculature as contemplated herein includes pathologies such as atherosclerosis and arteriosclerosis, cadiovascular complications of Type I and II diabetes, intimal hyperplasia, coronary heart disease, cerebral, coronary or arterial vasospasm, endothelial dysfunction, heart failure including congetive heart failure, sepsis, peripheral artery disease, restenosis and restenosis after angioplasty, stroke, vascular complications after organ transplantation, cardiovascular complications arising from viral and bacterial infections as well as any conditions which may be independent or secondary to another condition including mycardial infarction, hypertension, formation of atherosclerotic plaques, platelet aggregations, angina, aneurysm, transient ischemic attack, abnormal oxygen flow and/or delivery, atrophy or organ damage, pulmonary embolus, thrombotic or a generalized arterial or venous condition including endothelial dysfunction, a thrombotic event including deep vein thrombosis or damage to vessels of the circulatory system or stent failure or trauma caused by a stent, pacemaker or other prosthetic device as well as reperfusion injury including any injury caused after ischemia by restoration of blood flow and oxygen delivery, gangrene, (cancer and/or abnormal tumor), stem or progenitor cell proliferation, respiratory disease (eg. asthma, bronchitis, allergic rhinits and adult respiratory distress syndrome), skin disease (psoriasis, eczema and dermatitis), and various disorders of bone metabolisms (oestoporosis, hyperparathyroidism, oestosclorosis, oestoporasis and periodontits) and renal failure.

The present invention further contemplates a method of treating cancer or ameliorating the systems associated with cancer by the administration of an inhibitor of an NADPH oxidase comprising an extracellularly exposed Nox4. Examples of cancers contemplated herein include, without being limited to, ABL1 protooncogene, AIDS Related Cancers, Acoustic Neuroma, Acute Lymphocytic Leukaemia, Acute Myeloid Leukaemia, Adenocystic carcinoma, Adrenocortical Cancer, Agnogenic myeloid metaplasia, Alopecia, Alveolar soft-part sarcoma, Anal cancer, Angiosarcoma, Aplastic Anaemia, Astrocytoma, Ataxia-telangiectasia, Basal Cell Carcinoma (Skin), Bladder Cancer, Bone Cancers, Bowel cancer, Brain Stem Glioma, Brain and CNS Tumors, Breast Cancer, CNS tumors, Carcinoid Tumors, Cervical Cancer, Childhood Brain Tumors, Childhood Cancer, Childhood Leukaemia, Childhood Soft Tissue Sarcoma, Chondrosarcoma, Choriocarcinoma, Chronic Lymphocytic Leukaemia, Chronic Myeloid Leukaemia, Colorectal Cancers, Cutaneous T-Cell Lymphoma, Dermatofibrosarcoma-protuberans, Desmoplastic-Small-Round-Cell-Tumour, Ductal Carcinoma, Endocrine Cancers, Endometrial Cancer, Ependymoma, Esophageal Cancer, Ewing's Sarcoma, Extra-Hepatic Bile Duct Cancer, Eye Cancer, Eye: Melanoma, Retinoblastoma, Fallopian Tube cancer, Fanconi Anaemia, Fibrosarcoma, Gall Bladder Cancer, Gastric Cancer, Gastrointestinal Cancers, Gastrointestinal-Carcinoid-Tumour, Genitourinary Cancers, Germ Cell Tumors, Gestational-Trophoblastic-Disease, Glioma, Gynaecological Cancers, Haematological Malignancies, Hairy Cell Leukaemia, Head and Neck Cancer, Hepatocellular Cancer, Hereditary Breast Cancer, Histiocytosis, Hodgkin's Disease, Human Papillomavirus, Hydatidiform mole, Hypercalcemia, Hypopharynx Cancer, IntraOcular Melanoma, Islet cell cancer, Kaposi's sarcoma, Kidney Cancer, Langerhan's-Cell-Histiocytosis, Laryngeal Cancer, Leiomyosarcoma, Leukaemia, Li-Fraumeni Syndrome, Lip Cancer, Liposarcoma, Liver Cancer, Lung Cancer, Lymphedema, Lymphoma, Hodgkin's Lymphoma, Non-Hodgkin's Lymphoma, Male Breast Cancer, Malignant-Rhabdoid-Tumour-of-Kidney, Medulloblastoma, Melanoma, Merkel Cell Cancer, Mesothelioma, Metastatic Cancer, Mouth Cancer, Multiple Endocrine Neoplasia, Mycosis Fungoides, Myelodysplastic Syndromes, Myeloma, Myeloproliferative Disorders, Nasal Cancer, Nasopharyngeal Cancer, Nephroblastoma, Neuroblastoma, Neurofibromatosis, Nijmegen Breakage Syndrome, Non-Melanoma Skin Cancer, Non-Small-Cell-Lung-Cancer-(NSCLC), Ocular Cancers, Oesophageal Cancer, Oral cavity Cancer, Oropharynx Cancer, Osteosarcoma, Ostomy Ovarian Cancer, Pancreas Cancer, Paranasal Cancer, Parathyroid Cancer, Parotid Gland Cancer, Penile Cancer, Peripheral-Neuroectodermal-Tumors, Pituitary Cancer, Polycythemia vera, Prostate Cancer, Rare-cancers-and-associated-disorders, Renal Cell Carcinoma, Retinoblastoma, Rhabdomyosarcoma, Rothmund-Thomson Syndrome, Salivary Gland Cancer, Sarcoma, Schwannoma, Sezary syndrome, Skin Cancer, Small Cell Lung Cancer (SCLC), Small Intestine Cancer, Soft Tissue Sarcoma, Spinal Cord Tumors, Squamous-Cell-Carcinoma-(skin), Stomach Cancer, Synovial sarcoma, Testicular Cancer, Thymus Cancer, Thyroid Cancer, Transitional-Cell-Cancer-(bladder), Transitional-Cell-Cancer-(renal-pelvis-/-ureter), Trophoblastic Cancer, Urethral Cancer, Urinary System Cancer, Uroplakins, Uterine sarcoma, Uterus Cancer, Vaginal Cancer, Vulva Cancer, Waldenstrom's-Macroglobulinemia and Wilms' Tumour.

By the terms “effective amount” or “therapeutically effective amount” of an agent as used herein are meant a sufficient amount of the agent to provide the desired therapeutic effect. Furthermore, an “effective Nox4-inhibiting amount” or “an effective NADPH oxidase inhibiting amount” of an agent is a sufficient amount of the agent to at least partially inhibit or ameliorate the symptoms mediated or caused by ROS production. One particularly useful measure is the reduction in superoxide production and downstream ROS. Of course, undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

By “pharmaceutically acceptable” carrier excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emusifying agents, pH buffering agents, preservatives, and the like.

Similarly, a “pharmacologically acceptable” salt, ester, emide, prodrug or derivative of a compound as provided herein is a salt, ester, amide, prodrug or derivative that this not biologically or otherwise undesirable.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease. Thus, for example, the present method of “treating” a patient in need of therapy of the vascular system encompasses both prevention of a condition, disease or disorder as well as treating the condition, disease or disorder. In any event, the present invention contemplates the treatment or prophylaxis of any condition resulting in production or the likelihood of production of superoxide and/or downstream ROS by various cells such as cells of the vascular system and non-vascular system.

“Patient” as used herein refers to a mammalian, preferably human, individual who can benefit from the pharmaceutical formulations and methods of the present invention. There is no limitation on the type of mammal that could benefit from the presently described pharmaceutical formulations and methods. A patient regardless of whether a human or non-human mammal may be referred to as an individual, subject, mammal, host or recipient.

Accordingly, the present invention provides compounds which modulate NADPH oxidase function, activity or levels thereby influencing the extent to which the enzyme can generate superoxide and downstream ROS. Such ROS include hypochlorite, lipid peroxides, peroxynitrite, hydrogen peroxide, hydroxyl radicals. In particular, the compounds of the present invention selectively inhibit NADPH oxidase in vascular cells such as VSMCs and endothelial cells by specifically targeting extracellularly exposed Nox4 which is the NADPH-binding β-subunit of NADPH oxidase in those cells. In some non-vascular cells, such as leukocytes and phagocytes, the NADPH-binding β-subunit is gp91phox. Consequently, the compounds of the present invention inhibit or reduce levels of extracellularly exposed Nox4 but have substantially less of an effect or preferably no effect on gp91phox. This means that the compounds of the present invention can selectively inhibit direct or downstream ROS production in smooth muscle cell-containing vasculature and/or endothelial cell-containing vasculature and/or fibroblast-containing vasculature following an event of the vasculature. The compounds may be selective for Nox4 or may be selective in the sense that they are cell impermeable and hence are unable to inhibit intracellular Nox4 components or gp91phox components.

The compounds of this aspect of the present invention may be large or small molecules, nucleic acid molecules, peptides, polypeptides or proteins or antibodies or hybrid molecules such as RNAi-complexes, ribozymes or DNAzymes.

Another aspect of the present invention provides a compound capable of interacting with a polypeptide comprising a sequence of amino acids set forth in SEQ ID NO:2 or SEQ ID NO:4 or having at least about 50% similarity thereto or interacting with a nucleic acid molecule comprising a nucleotide sequence as set forth in SEQ ID NO:1 or SEQ ID NO:3 or its complement or having at least about 50% identity to SEQ ID NO:1 or SEQ ID NO:3 or its complement or a nucleotide sequence capable of hybridizing to SEQ ID NO:1 or SEQ ID NO:3 or its complementary form under low stringency conditions wherein said compound acts as an antagonist of said polypeptide activity or function or expression of said nucleic acid molecules.

SEQ ID NO:2 represents the amino acid sequence of human Nox4. SEQ ID NO:1 is the nucleotide sequence encoding human Nox4.

The nucleotide sequence encoding mouse Nox4 is defined by SEQ ID NO:3. The corresponding amino acid sequence is defined by SEQ ID NO:4.

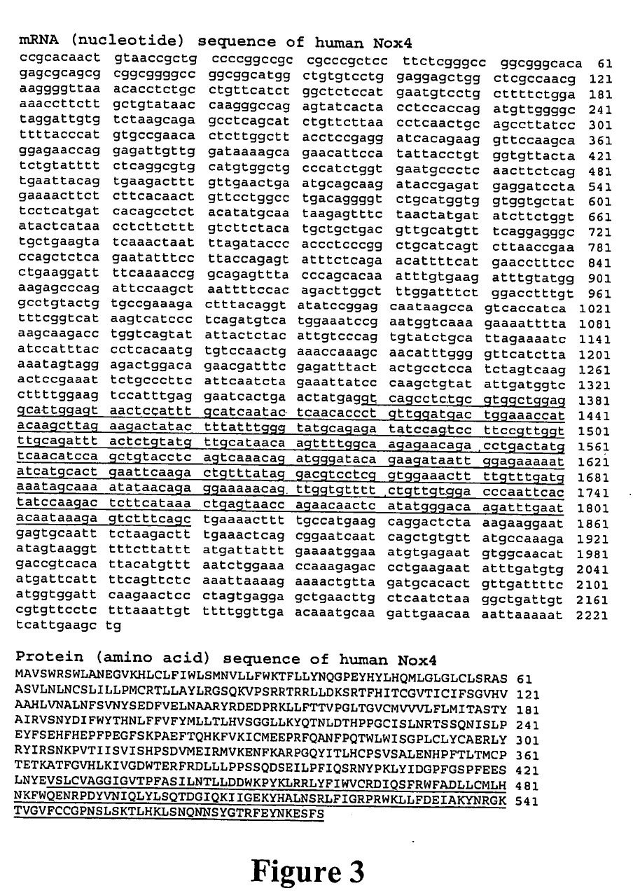

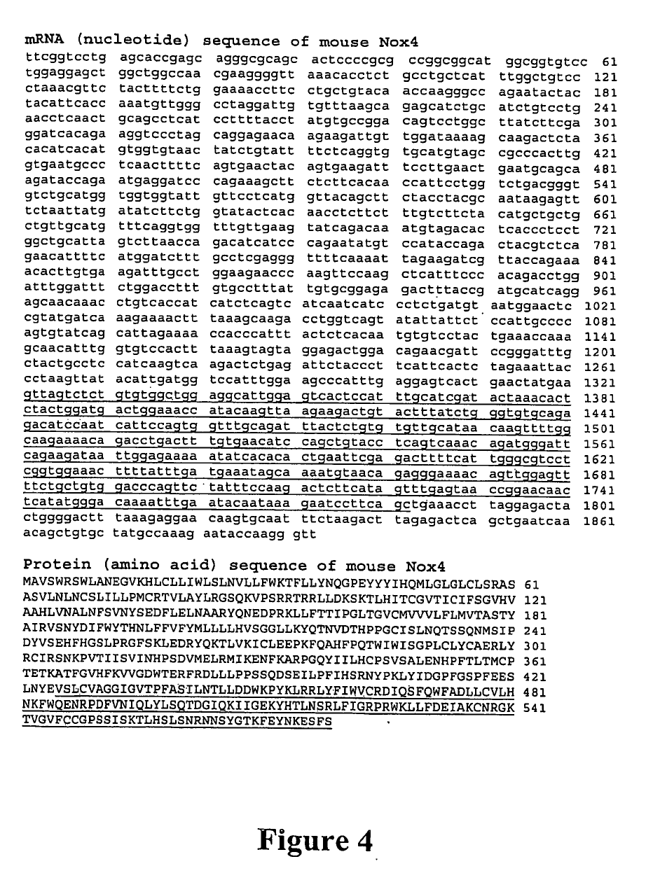

FIGS. 3 and 4 show the nucleotide and amino acid sequences of human and mouse Nox4, respectively. Importantly, the predicted extracellular domain of Nox4 is underlined (corresponding to SEQ ID NOs: 5 and 7, respectively and encoded by SEQ ID NOs:4 and 6).

Consequently, another aspect of the present invention provides a compound capable of interacting with a polypeptide comprising a sequence of amino acids set forth in SEQ ID NO:5 or SEQ ID NO:7 or having at least about 50% similarity thereto or interacting with a nucleic acid molecule comprising a nucleotide sequence as set forth in SEQ ID NO:4 or SEQ ID NO:6 or its complement or having at least about 50% identity to SEQ ID NO:4 or SEQ ID NO:6 or its complement or a nucleotide sequence capable of hybridizing to SEQ ID NO:4 or SEQ ID NO:6 or its complementary form under low stringency conditions wherein said compound acts as an antagonist of said polypeptide activity or function or expression of said nucleic acid molecules.

The present invention extends, however, to the targeting of Nox4 from any mammalian source such as from other primates, livestock animals, laboratory test animals, companion animals or captive wild animals.

Examples of laboratory test animals include mice, rats, rabbits, guinea pigs and hamsters. Rabbits and rodent animals, such as rats and mice, provide a convenient test system or animal model. Livestock animals include sheep, cows, pigs, goats, horses and donkeys. Non-mammalian animals such as zebrafish and amphibians (including cane toads) may also be a useful model.

The terms “similarity” or identity as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and amino acid sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, 1994-1998, Chapter 15).

The terms “sequence similarity” and “sequence identity” as used herein refer to the extent that sequences are identical or functionally or structurally similar on a nucleotide-by-nucleotide basis or an amino acid-by-amino acid basis over a window of comparison.

Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Preferably, the percentage similarity between a particular sequence and a reference sequence (nucleotide or amino acid) is at least about 60% or at least about 70% or at least about 80% or at least about 90% or at least about 95% or above such as at least about 96%, 97%, 98%, 99% or greater. Percentage similarities or identities between 50 and 100 are also contemplated.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The terms “nucleic acids”, “nucleotide” and “polynucleotide” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

The present invention extends to a portion or part or fragment of the Nox4 gene or its mRNA. A “portion or part or fragment” is defined as having a minimal size of at least about 8 nucleotides or preferably about 12-17 nucleotides or more preferably at least about 18-25 nucleotides and may have a maximal size of at least about 5000 nucleotides. Genomic equivalents larger than 5000 nucleotides may also be employed. This definition includes all sizes in the range of 8-5000 nucleotides. Thus, this definition includes nucleic acids of 12, 15, 20, 25, 40, 60, 80, 100, 200, 300, 400, 500 or 1000 nucleotides or nucleic acids having any number of nucleotides within these values (e.g. 13, 16, 23, 30, 28, 50, 72, 121, etc. nucleotides) or nucleic acids having more than 500 nucleotides or any number of nucleotides between 500 and the number shown in SEQ ID NO:1. The present invention includes all novel nucleic acids having at least 8 nucleotides derived from SEQ D NO: 1 or a complement or functional equivalent thereof.

The present invention provides methods of screening for drugs comprising, for example, contacting a prodrug with a Nox4 polypeptide or fragment thereof and assaying (i) for the presence of a complex between the drug and the Nox4 polypeptide or fragment, or (ii) for the presence of a complex between the Nox4 polypeptide or fragment and a ligand, by methods well known in the art. In such competitive binding assays, the Nox4 polypeptide or fragment is typically labeled. Free Nox4 polypeptide or fragment is separated from that present in a protein:protein complex and the amount of free (i.e. uncomplexed) label is a measure of the binding of the agent being tested to Nox4. One may also measure the amount of bound, rather than free, Nox4. It is also possible to label the ligand rather than the Nox4 and to measure the amount of ligand binding to Nox4 in the presence and in the absence of the drug being tested.

Another technique for drug screening provides high throughput screening for compounds having suitable binding affinity to Nox4 and is described in detail in Geysen (International Patent Publication No. WO 84/03564). Briefly stated, large numbers of different small peptide test compounds are synthesized on a solid substrate, such as plastic pins or some other surface. The peptide test compounds are reacted with Nox4 and washed. Bound Nox4 polypeptide is then detected by methods well known in the art. This method may be adapted for screening for non-peptide, chemical entities. This aspect, therefore, extends to combinatorial approaches to screening for Nox4 antagonists.

Purified Nox4 can be coated directly onto plates for use in the aforementioned drug screening techniques. However, non-neutralizing antibodies to the polypeptide can be used to capture antibodies to immobilize the Nox4 polypeptide on the solid phase.

The present invention also contemplates the use of competitive drug screening assays in which neutralizing antibodies capable of specifically binding the Nox4 polypeptide compete with a test compound for binding to the Nox4 polypeptide or fragments thereof.

In this manner, the antibodies can be used to detect the presence of any peptide which shares one or more antigenic determinants of the Nox4 polypeptide.

The above screening methods are not limited to assays employing only Nox4 but are also applicable to studying Nox4-protein complexes such as intact NADPH oxidase or membrane preparations comprising same. The effect of drugs on the activity of this complex is analyzed.

Furthermore, a range of assays are contemplated based on the discovery herein that at least a portion of the Nox4 component is extracellular or at least exists in equilibrium between external, internal or intraternal. This is considered further below.

In accordance with the present invention, benzamides and/or aryl sulphonates and/or derivatives or analogs and in particular sulfated benzamide and aryl sulphonates and derivatives or analogs are proposed to selectively inhibit Nox4. One particularly useful aryl sulphonate and derivative or analog for the practice of the present invention is suramin and its derivatives, analogs and functional homologs.

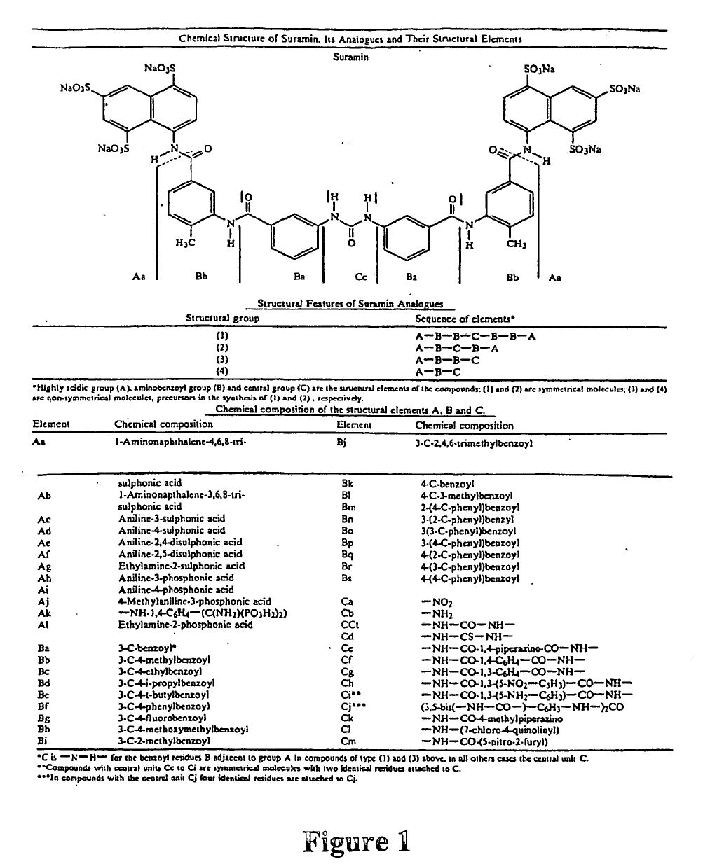

The structure of suramin is presented in FIG. 1. Reference herein to suramin includes the analogs disclosed by Jentsch et al., 1987, supra. A summary of these analogs is shown in FIG. 1 [Jentsch et al., 1987, supra; U.S. Pat. No. 5,173,509].

Additionally, the specific active suramin related analogs encompassed hereby and disclosed by Jentsch et al., 1987, supra in Tables 3 and 4 thereof at pages 2187 and 2188 have the structures and molecular weights as shown in Table 3:

TABLE 3

| Compound No. | Structure¹ | Molecular Weight² |
|---|---|---|
| 1³ | (Aa-Bb-Ba-)₂Cc | 1429.2 |
| 2 | (Ai-Ba-)₂Cc | 698.4 |
| 3 | (Ab-Bk-Bk-)₂Cc | 1401.1 |
| 4 | (Ab-Ba-Ba-)₂Cc | 1401.1 |
| 5 | (Aa-)₄Cj | 1096.2 |
| 6 | (Aa-Bg-)₂Cc | 1198.2 |
| 7 | (Aa-Bi-Ba-)₂Cc | 1429.2 |
| 8 | (Aa-Bn-)₂Cc | 1315.1 |
| 9 | (Ab-Bk-Ba-)₂Cc | 718.6 |
| 10 | (Aa-Bc-)₂Cc | 1219.0 |
| 11 | (Aa-Bb-)₂Cg | 1295.1 |
| 12 | (Aa-Bb-Ba-)₂Cf | 1535.5 |
| 13 | (Aa-Bb-)₂Cc | 1191.0 |
| 14 | (Aa-Bs-)₂Cc | 1315.1 |
| 15 | (Ab-)Cl | 610.5 |
| 16 | (Ab-Bk-Ba-)₂Cc | 1401.1 |
| 17 | (Ah-Bk-)₂Cc | 654.4 |
| 18 | (Aa-Bc-Ba-)₂Cc | 1457.9 |
| 19 | (Aa-Bi-)₂Cc | 1191.0 |
| 20 | (Aa-Ba-)₂Cc | 1162.0 |
| 21 | (Ai-Bk-)₂Cc | 698.4 |
| 22 | (Aa-)Cl | 610.5 |
| 23 | (Aa-Bb-Ba-)₂Cg | 1535.3 |
| 24 | (Af-Bb-Ba-)₂Cc | 674.6 |
| 25 | Aa-Bs-Ca | 1060.9 |
| 26 | Ab-Bk-Bk-Ca | 717.6 |
| 27 | (Ae-Bb-Ba-)₂Cc | 1060.9 |
| 28 | (Ah-Ba-)₂Cc | 654.5 |
| 29 | Ab-Bk-Bk-Ca | 687.6 |
| 30 | Aa-Bs-Cb | 644.5 |
| 31 | Aa-Bb-Cl | 730.5 |
| 32 | (Ac-Bb-Ba-)₂Ce | 920.9 |
| 33 | (Aa-Ba-Bb-)₂Cc | 1429.2 |
| 34 | (Aa-Bd-)₂Cc | 1247.1 |
| 35 | (Aa-Bd-Bd-Bd-)₂Ce | 1723.6 |
| 36 | (Aa-Bh-Ba-)₂Cc | 1489.3 |
| 37 | (Aa-Bb-Bb-)₂Ce | 1457.3 |
| 38 | (Aa-Bb-)₂Cc | 1301.1 |
| 39 | (Aa-Bb-)₂Ce | 1485.4 |
| 40 | (Aa-bj-Ba-)₂Ce | 1162.8 |
| 41 | (Ab-Bk-)₂Ce | 1429.2 |
| 42 | (Aa-Bl-Ba-)₂Ce | 1251.0 |
| 43 | (Aa-Bh-)₂Cc | 1191.0 |
| 44 | (Aa-Br-)₂Ce | 1351.1 |
| 45 | (Aa-Bg-Ba-)₂Ce | 1477.1 |
| 46 | (Aa-Bb-)Cm | 721.6 |
| 47 | (Aa-Bq-) | 1351.1 |
| 48 | (Aa-Bc-)₂Ce | 1275.1 |
| 49 | (Aa-Bm-)₂Ce | 1315.1 |
| 50 | (Aa-Be-Ba-)₂Ce | 1513.4 |
| 51 | (Aa-Bd-Ba-)₂Ce | 1485.4 |
| 52 | (Aa-Bf-)₂Cc | 1315.1 |
| 53 | (Aa-Bf-Ba-)₂Ce | 1553.4 |
| 54 | (Aa-Bj-)₂Ce | 1245.0 |
| 55 | (Aa-)Cm | 588.2 |
| 56 | (Ai-Bk-)₂Cd | 714.5 |
| 57 | (Ah-Ba-)₂Cd | 670.7 |

¹Synthesis of each of the suramin analogs (compound numbers 2-57) have been previously reported [Nickel et al., Arzneimittel-Forschung 36: 1153-57, 1986] and Holzmann et al., Biomedical Mass Spectrophotometry 12: 659-663, 1985]. A, B and C structural units are as defined above.
²Molecular weight of the sodium salt.
³Suramin (sodium salt)

In particular, the suramin binding sites on human and murine Nox4 are highlighted in FIGS. 3 and 4, respectively.

Accordingly, the present invention contemplates any compound which binds or otherwise interacts with the extracellularly exposed suramin or NADPH binding site as defined in FIG. 3 (human) and FIG. 4 (mouse) or its functional equivalent in other mammalian or non-mammalian animals.

The identification of suramin as a Nox4 antagonist enables strategies to be developed for determining the location of suramin binding site on Nox4. This enables the generation of agonists (i.e. potentiators) of suramin-Nox4 interaction as well as identifying other like antagonists.

In one approach, superoxide or other ROS production is demonstrated in VSMCs stimulated with NADPH. It is then shown that, under the same conditions, the addition of suramin blocks superoxide or other ROS production. VSMCs are then incubated with labeled suramin such as fluorescein-labeled suramin and NADPH-stimulated superoxide (or other ROS) production is measured using chemiluminescence to ensure that labeling has not affected the inhibitory activity of suramin. This being the case, the VSMCs are then visualized under high power using a confocal or fluoroescence microscope to demonstrate that the labeled suramin is bound to an extracellular site and has not penetrated the plasma membrane.

Epitope tagging is another approach. Suramin is an NADPH analog and may inhibit NADPH oxidase activity by occupying the NADPH binding site of the Nox4 subunit. Since the NADPH binding site of Nox4 is located on its C-terminal tail, epitope-tagging of this region and subsequent analysis of antibody binding in intact versus permeabilised cells enables determination of whether it is located on the intracellular or extracellular surface of the plasma membrane.

Total RNA is extracted from VSMCs and reverse transcribed using poly dT primers. The resulting cDNA is then used as a template to amplify the entire coding domain of Nox4 by polymerase chain reaction (PCR). The PCR product is inserted immediately upstream from a FLAG epitope in a GATEWAY (Reg. Trademark) expression vector (Invitroge, Calif., USA) or other suitable epitope-containing vector. The construct is then transiently expressed in suitable cells and binding of an antibody against the FLAG epitope (or other epitope) is then compared in intact versus permeabilized cells using confocal or fluorescence microscopy.

Other useful drugs which act in a similar manner to suramin are Reactive blue-2 and PPADS. Reactive blue-2 (also known as Basilen blue E-3G, Cibacron blue F3G-A and Procion blue H-B) is [1-amino-4[[4[[4-chloro-6-[[n-sulfophenyl]amino]-1,3,5-triazin-2-yl]amino]-3-sulfophenyl]amino]-9,10-dihydro-9,10-dioxo-2-anthracene-sulfonic acid wherein n is 3 or 4]. PPADS is 4-[[-formyl-5-hydroxy-6-methyl-3-[(phos-phonooxy)methyl]-2-pyridinyl]azo]-1,3-benzenedisulfonic acid.

Further useful drugs include superoxide scavengers such as tempol (4-hydroxy-2,2,6,6-tetramethyl piperidinoxyl) and compounds which block superoxide formation from NADPH oxidase such as suramin (described above), diphenyleneiodonium (DPI) and apocynin as well as molecules which bind to an extracellular portion of Nox4 thereby scavanging ROS.

The present invention is also useful for screening for other compounds which inhibit the Nox4 polypeptide. The Nox4 polypeptide or binding fragment thereof may be used in any of a variety of drug screening techniques, such as those described herein and in International Publication No. WO 97/02048.

A Nox4 antagonist includes a Nox4 variant polypeptide. The term “polypeptide” refers to a polymer of amino acids and its equivalent and does not refer to a specific length of the product, thus, peptides, oligopeptides and proteins are included within the definition of a polypeptide. This term also does not refer to or exclude modifications of the polypeptide, for example, glycosylations, aceylations, phosphorylations and the like. Included within the definition are, for example, polypeptides containing one or more analogs of an amino acid (including, for example, unnatural amino acids, etc.), polypeptides with substituted linkages as well as other modifications known in the art, both naturally and non-naturally occurring. Ordinarily, such polypeptides will be at least about 40% similar to the natural Nox4 sequence, preferably in excess of 90% and more preferably at least about 95% similar. Also included are proteins encoding by DNAs which hybridize under high or low stringency conditions to Nox4-encoding nucleic acids and closely related polypeptides or proteins retrieved by antisera to the Nox4 protein.

Substitutional variants typically contain the exchange of one amino acid for another at one or more sites within the protein and may be designed to modulate one or more properties of the polypeptide such as stability against proteolytic cleavage without the loss of other functions or properties. Amino acid substitutions may be made on the basis of similarity in polarity, charge, solubility, hydrophobicity, hydrophilicity and/or the amphipathic nature of the residues involved. Preferred substitutions are ones which are conservative, that is, one amino acid is replaced with one of similar shape and charge. Conservative substitutions are well known in the art and typically include substitutions within the following groups: glycine, alanine; valine, isoleucine, leucine; aspartic acid, glutamic acid; asparagine, glutamine; serine, threonine; lysine, arginine; and tyrosine, phenylalanine.

Certain amino acids may be substituted for other amino acids in a protein structure without appreciable loss of interactive binding capacity with structures such as, for example, antigen-binding regions of antibodies or binding sites on substrate molecules or binding sites on proteins interacting with the Nox4 polypeptide. Since it is the interactive capacity and nature of a protein which defines that protein's biological functional activity, certain amino acid substitutions can be made in a protein sequence and its underlying DNA coding sequence and nevertheless obtain a protein with like properties. In making such changes, the hydropathic index of amino acids may be considered. The importance of the hydrophobic amino acid index in conferring interactive biological function on a protein is generally understood in the art (Kyte and Doolittle, J. Mol. Biol. 157: 105-132, 1982). Alternatively, the substitution of like amino acids can be made effectively on the basis of hydrophilicity. The importance of hydrophilicity in conferring interactive biological function of a protein is generally understood in the art (U.S. Pat. No. 4,554,101). The use of the hydrophobic index or hydrophilicity in designing polypeptides is further discussed in U.S. Pat. No. 5,691,198.

The length of the polypeptide sequences compared for homology will generally be at least about 16 amino acids, usually at least about 20 residues, more usually at least about 24 residues, typically at least about 28 residues and preferably more than about 35 residues.

The present invention further contemplates chemical analogs of the Nox4 polypeptide. Again, these are generally antagonistic to Nox4 activity.

Analogs contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 4.

TABLE 4
Codes for non-conventional amino acids

| | | Non-conventional | |
|---|---|---|---|
| Non-conventional amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

The term “peptide mimetic” or “mimetic” is intended to refer to a substance which has some chemical similarity to Nox4 but which antagonizes the Nox4 polypeptide. A peptide mimetic may be a peptide-containing molecule that mimics elements of protein secondary structure (Johnson et al., “Peptide Turn Mimetics” in Biotechnology and Pharmacy, Pezzuto et al., Eds., Chapman and Hall, New York, 1993). The underlying rationale behind the use of peptide mimetics is that the peptide backbone of proteins exists chiefly to orient amino acid side chains in such a way as to facilitate molecular interactions such as those of antibody and antigen, enzyme and substrate or scaffolding proteins. A peptide mimetic is designed to permit molecular interactions similar to the natural molecule and, hence, compete for molecules which might otherwise generate ROS with the naturally occurring Nox4.

Again, the compounds of the present invention may be selected to target Nox4 alone or single or multiple compounds may be used to target Nox4 and one or more other NADPH oxidase components.

The Nox4 polypeptide or fragment employed in such a test may either be free in solution, affixed to a solid support, or borne on a cell surface. One method of drug screening utilizes eukaryotic or procaryotic host cells which are stably transformed with recombinant polynucleotides expressing the polypeptide or fragment, preferably in competitive binding assays. Such cells, either in viable or fixed form, can be used for standard binding assays. One may measure, for example, the formation of complexes between a Nox4 polypeptide or fragment and the agent being tested, or examine the degree to which the formation of a complex between a Nox4 polypeptide or fragment and a known ligand is aided or interfered with by the agent being tested.

The above methods and the methods further described below are also applicable to identifying agonists of Nox4-inhibitor interaction such as suramin-Nox4 interaction. Such agonists may be used in conjunction with suramin or other Nox4 inhibitors to enhance the inhibitory effects. These agonists act as potentiators of compounds which inhibit Nox4.

Antisense polynucleotide sequences are useful in preventing or diminishing the expression of the Nox4 locus, as will be appreciated by those skilled in the art. Polynucleotide vectors, for example, containing all or a portion of the Nox4 locus or other sequences from the Nox4 region (particularly those flanking the Nox4 locus) may be placed under the control of a promoter in an antisense orientation and introduced into a cell. Expression of such an antisense construct within a cell will interfere with Nox4 transcription and/or translation. Furthermore, co-suppression and mechanisms to induce RNAi (i.e. siRNA) may also be employed. Such techniques may be useful to inhibit genes which positively promote Nox4 expression. Alternatively, antisense or sense molecules may be administered directly. In this latter embodiment, the antisense or sense molecules may be formulated in a composition and then administered by any number of means to target cells.

A variation on antisense and sense molecules involves the use of morpholinos, which are oligonucleotides composed of morpholine nucleotide derivatives and phosphorodiamidate linkages (for example, Summerton and Weller, Antisense and Nucleic Acid Drug Development 7: 187-195, 1997). Such compounds are injected into embryos and the effect of interference with mRNA is observed.

In one embodiment, the present invention employs compounds such as oligonucleotides and similar species for use in modulating the function or effect of nucleic acid molecules encoding Nox4, i.e. the oligonucleotides induce transcriptional or post-transcriptional gene silencing. This is accomplished by providing oligonucleotides which specifically hybridize with one or more nucleic acid molecules encoding Nox4. As used herein, the terms “target nucleic acid” and “nucleic acid molecule encoding Nox4” have been used for convenience to encompass DNA encoding Nox4, RNA (including pre-mRNA and mRNA or portions thereof) transcribed from such DNA, and also cDNA derived from such RNA. The hybridization of a compound of the subject invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. One preferred result of such interference with target nucleic acid function is modulation of the expression of the Nox4 gene. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is often the preferred form of modulation of expression and mRNA is often a preferred target nucleic acid.

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e. under physiological conditions in the case of in vivo assays or therapeutic treatment, and under conditions in which assays are performed in the case of in vitro assays.

“Complementary” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other.

Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

According to the present invention, compounds include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these compounds may be introduced in the form of single-stranded, double-stranded, circular or hairpin oligomeric compounds and may contain structural elements such as internal or terminal bulges or loops. Once introduced to a system, the compounds of the invention may elicit the action of one or more enzymes or structural proteins to effect modification of the target nucleic acid. One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals.

In the context of the subject invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the compounds of this invention, the present invention comprehends other families of compounds as well, including but not limited to oligonucleotide analogs and mimetics such as those described herein.

The compounds in accordance with this invention preferably comprise from about 8 to about 80 nucleobases (i.e. from about 8 to about 80 linked nucleosides). One of ordinary skill in the art will appreciate that the invention embodies compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

The open reading frame (ORF) or “coding region” which is known in the art to refer to the region between the translation initiation codon and the translation termination codon, is a region which may be targeted effectively. Within the context of the present invention, one region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns”, which are excised from a transcript before it is translated. The remaining (and, therefore, translated) regions are known as “exons” and are spliced together to form a continuous mRNA sequence. Targeting splice sites, i.e. intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts”. It is also known that introns can be effectively targeted using antisense compounds targeted to, for example, DNA or pre-mRNA.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts, mixed salts and free acid forms are also included.

Many of the preferred features described above are appropriate for sense nucleic acid molecules.

The present invention extends to antisense and other nucleic acid molecules directed to other genes such as other portions of NADPH oxidase unique to particular target cells compared to other cells.

Following identification of a substance which modulates or affects polypeptide activity or gene expression or mRNA translation and/or which agonize (i.e. potentiate) the interaction between an inhibitor and Nox4, the substance may be further investigated. Furthermore, it may be manufactured and/or used in preparation, i.e. manufacture or formulation or a composition such as a medicament, pharmaceutical composition or drug. These may be administered to individuals in a method of treatment or prophylaxis. Alternatively, they may be incorporated into a patch, slow release capsule or implant or stent or other device inserted into vessels or tissue such as a catheter.

Thus, the present invention extends, therefore, to a pharmaceutical composition, medicament, drug or other composition including a stent, catheter, patch or slow release formulation comprising an antagonist of Nox4 activity or gene expression. Preferably, the medicament or drug is cell impermeable. Alternatively, it is selective for Nox4. In addition, the pharmaceutical composition may further contain an agonist of Nox4-inhibitor interaction or the agonist may be in a separate composition Another aspect of the present invention contemplates a method comprising administration of such a composition to a patient such as for treatment or prophylaxis of an event or condition of the systemic vasculature such as atherosclerosis or endothelial dysfunction. The compounds of the present invention may also be used in the manufacture of a medicament for the treatment or prophylaxis of an event or condition of the systemic vasculature. Furthermore, the present invention contemplates a method of making a pharmaceutical composition comprising admixing a compound of the instant invention with a pharmaceutically acceptable excipient, vehicle or carrier, and optionally other ingredients. Where multiple compositions are provided, such as with a Nox4 inhibitor and an agonist of Nox4-inhibitor interaction, then such compositions may be given simultaneously or sequentially. Sequential administration includes administration within nanoseconds, seconds, minutes, hours or days. Preferably, within seconds or minutes.

Such compositions are proposed to be useful in the treatment and/or prophylaxis of and pathologies such as atherosclerosis and arteriosclerosis, cadiovascular complications of Type I and II diabetes, intimal hyperplasia, coronary heart disease, cerebral, coronary or arterial vasospasm, endothelial dysfunction, heart failure including congetive heart failure, sepsis, peripheral artery disease, restenosis and restenosis after angioplasty, stroke, vascular complications after organ transplantation, cardiovascular complications arising from viral and bacterial infections as well as any conditions which may be independent or secondary to another condition including mycardial infarction, hypertension, formation of atherosclerotic plaques, platelet aggregations, angina, aneurysm, transient ischemic attack, abnormal oxygen flow and/or delivery, atrophy or organ damage, pulmonary embolus, thrombotic or a generalized arterial or venous condition including endothelial dysfunction, a thrombotic event including deep vein thrombosis or damage to vessels of the circulatory system or stent failure or trauma caused by a stent, pacemaker or other prosthetic device as well as reperfusion injury including any injury caused after ischemia by restoration of blood flow and oxygen delivery, gangrene, (cancer and/or abnormal tumor), stem or progenitor cell proliferation, respiratory disease (eg. asthma, bronchitis, allergic rhinits and adult respiratory distress syndrome), skin disease (psoriasis, eczema and dermatitis), and various disorders of bone metabolisms (oestoporosis, hyperparathyroidism, oestosclorosis, oestoporasis and periodontits) and renal failure.

The subject formulations may also be in the form of multicomponent pharmaceutical compositions comprising a Nox4 antagonist or antagonist of an extracellurlarly exposed portion of NADPH oxidase (eg. all or part of Nox4) and one or more agents selected from cholesterol lowering agents, antihypertensive agents, antidiabetic agents, antioxidants and anti-arrhythmic agents. Such formulations may also be referred to as multi-pharmaceutical packs and the individual active agents may be formulated together or admixed prior to use. Alternatively, they may be separately administered within seconds, minutes, hours, days or weeks of each other.

Accordingly, another aspect of the present invention contemplates a method for the treatment or prophylaxis of a condition in a mammal, said method comprising administering to said mammal an effective amount of a compound as described herein or a composition comprising same. Generally, the condition involves or is caused by ROS production by a Nox4-containing NADPH oxidase.

Preferably, the mammal is a human or laboratory test animal such as a mouse, rat, rabbit, guinea pig, hamster, zebrafish or amphibian. Conditions contemplated herein include pathologies such as atherosclerosis and arteriosclerosis, cadiovascular complications of Type I and II diabetes, intimal hyperplasia, coronary heart disease, cerebral, coronary or arterial vasospasm, endothelial dysfunction, heart failure including congetive heart failure, sepsis, peripheral artery disease, restenosis and restenosis after angioplasty, stroke, vascular complications after organ transplantation, cardiovascular complications arising from viral and bacterial infections as well as any conditions which may be independent or secondary to another condition including mycardial infarction, hypertension, formation of atherosclerotic plaques, platelet aggregations, angina, aneurysm, transient ischemic attack, abnormal oxygen flow and/or delivery, atrophy or organ damage, pulmonary embolus, thrombotic or a generalized arterial or venous condition including endothelial dysfunction, a thrombotic event including deep vein thrombosis or damage to vessels of the circulatory system or stent failure or trauma caused by a stent, pacemaker or other prosthetic device as well as reperfusion injury including any injury caused after ischemia by restoration of blood flow and oxygen delivery, gangrene, (cancer and/or abnormal tumor), stem or progenitor cell proliferation, respiratory disease (eg. asthma, bronchitis, allergic rhinits and adult respiratory distress syndrome), skin disease (psoriasis, eczema and dermatitis), and various disorders of bone metabolisms (oestoporosis, hyperparathyroidism, oestosclorosis, oestoporasis and periodontits) and renal failure.

A substance identified as a modulator of polypeptide function or gene activity may be a peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This might be desirable where the active compound is difficult or expensive to synthesize or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g. by substituting each residue in turn. Alanine scans of peptides are commonly used to refine such peptide motifs. These parts or residues constituting the active region of the compound are known as its “pharmacophore”.

Once the pharmacophore has been found, its structure is modeled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modeling process.

In a variant of this approach, the three-dimensional structure of the ligand and its binding partner are modeled. This can be especially useful where the ligand and/or binding partner change conformation on binding, allowing the model to take account of this in the design of the mimetic. The NADPH binding site on human and mouse Nox4 is shown in FIGS. 3 and 4, respectively. Modeling can be used to generate inhibitors which interact with the linear sequence or a three-dimensional configuration.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

The goal of rational drug design is to produce structural analogs of biologically active polypeptides of interest or of small molecules with which they interact (e.g. agonists, antagonists, inhibitors or enhancers) in order to fashion drugs which are, for example, more active or stable forms of the polypeptide, or which, e.g. enhance or interfere with the function of a polypeptide in vivo. See, e.g. Hodgson (Bio/Technology 9: 19-21, 1991). In one approach, one first determines the three-dimensional structure of a protein of interest (i.e. Nox4) by x-ray crystallography, by computer modeling or most typically, by a combination of approaches. Useful information regarding the structure of a polypeptide may also be gained by modeling based on the structure of homologous proteins. An example of rational drug design is the development of HIV protease inhibitors (Erickson et al., Science 249: 527-533, 1990). In addition, Nox4 may be analyzed by an alanine scan (Wells, Methods Enzymol. 202: 2699-2705, 1991). In this technique, an amino acid residue is replaced by Ala and its effect on the peptide's activity is determined. Each of the amino acid residues of the peptide is analyzed in this manner to determine the important regions of the peptide.

It is also possible to isolate a target-specific antibody, selected by a functional assay and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacore.

Again, similar methods may be used to identify compounds which potentiate the inhibitory effect of other compounds on Nox4. The potentiators may also be referred to herein as agonists of Nox4 inhibitors.

Thus, one may design drugs which have antagonistic activity towards Nox4 or Nox4 gene expression.

According to the present invention, a method is also provided of supplying wild-type or mutant Nox4 gene function to a cell. This is particularly useful when generating an animal model which highlight the effects of ROS production in VSMCs as well as other cells.

Alternatively, it may be part of a gene therapy approach. The Nox4 gene or a part of the gene may be introduced into the cell in a vector such that the gene remains extrachromosomal. In such a situation, the gene will be expressed by the cell from the extrachromosomal location. If a gene portion is introduced and expressed in a cell carrying a mutant Nox4 allele, the gene portion should encode a part of the Nox4 protein. Vectors for introduction of genes both for recombination and for extrachromosomal maintenance are known in the art and any suitable vector may be used. Methods for introducing DNA into cells such as electroporation calcium phosphate co-precipitation and viral transduction are known in the art.

Gene transfer systems known in the art may be useful in the practice of genetic manipulation. These include viral and non-viral transfer methods. A number of viruses have been used as gene transfer vectors or as the basis for preparing gene transfer vectors, including papovaviruses (e.g. SV40, Madzak et al., J. Gen. Virol. 73: 1533-1536, 1992), adenovirus (Berkner, Curr. Top. Microbiol. Immunol. 158: 39-66, 1992; Berkner et al., BioTechniques 6; 616-629, 1988; Gorziglia and Kapikian, J. Virol. 66: 4407-4412, 1992; Quantin et al., Proc. Natl. Acad. Sci. USA 89: 2581-2584, 1992; Rosenfeld et al., Cell 68: 143-155, 1992; Wilkinson et al., Nucleic Acids Res. 20: 2233-2239, 1992; Stratford-Perricaudet et al., Hum. Gene Ther. 1: 241-256, 1990; Schneider et al., Nature Genetics 18: 180-183, 1998), vaccinia virus (Moss, Curr. Top. Microbiol. Immunol. 158: 25-38, 1992; Moss, Proc. Natl. Acad. Sci. USA 93: 11341-11348, 1996), adeno-associated virus (Muzyczka, Curr. Top. Microbiol. Immunol. 158: 97-129, 1992; Ohi et al., Gene 89: 279-282, 1990; Russell and Hirata, Nature Genetics 18: 323-328, 1998), herpesviruses including HSV and EBV (Margolskee, Curr. Top., Microbiol. Immunol. 158: 67-95, 1992; Johnson et al., J. Virol. 66: 2952-2965, 1992; Fink et al., Hum. Gene Ther. 3: 11-19, 1992; Breakefield and Geller, Mol. Neurobiol. 1: 339-371, 1987; Freese et al., Biochem. Pharmacol. 40: 2189-2199, 1990; Fink et al., Ann. Rev. Neurosci. 19: 265-287, 1996), lentiviruses (Naldini et al, Science 272: 263-267, 1996), Sindbis and Semliki Forest virus (Berglund et al., Biotechnology 11: 916-920, 1993) and retroviruses of avian (Bandyopadhyay and Temin, Mol. Cell. Biol. 4: 749-754, 1984; Petropoulos et al., J. Viol. 66: 3391-3397, 1992], murine [Miller, Curr. Top. Microbiol. Immunol. 158: 1-24, 1992; Miller et al., Mol. Cell. Biol. 5: 431-437, 1985; Sorge et al., Mol. Cell. Biol. 4: 1730-1737, 1984; Mann and Baltimore, J. Virol. 54: 401-407, 1985; Miller et al., J. Virol. 62: 4337-4345, 1988) and human [Shimada et al., J. Clin. Invest. 88: 1043-1047, 1991; Helseth et al., J. Virol. 64: 2416-2420, 1990; Page et al., J. Virol. 64: 5270-5276, 1990; Buchschacher and Panganiban, J. Virol. 66: 2731-2739, 1982] origin.

Non-viral gene transfer methods are known in the art such as chemical techniques including calcium phosphate co-precipitation, mechanical techniques, for example, microinjection, membrane fusion-mediated transfer via liposomes and direct DNA uptake and receptor-mediated DNA transfer. Viral-mediated gene transfer can be combined with direct in vivo gene transfer using liposome delivery, allowing one to direct the viralvectors to the tumor cells and not into the surrounding non-dividing cells. Alternatively, the retroviral vector producer cell line can be injected into tumors. Injection of producer cells would then provide a continuous source of vector particles.

In an approach which combines biological and physical gene transfer methods, plasmid DNA of any size is combined with a polylysine-conjugated antibody specific to the adenovirus hexon protein and the resulting complex is bound to an adenovirus vector. The trimolecular complex is then used to infect cells. The adenovirus vector permits efficient binding, internalization and degradation of the endosome before the coupled DNA is damaged. For other techniques for the delivery of adenovirus based vectors, see U.S. Pat. No. 5,691,198.

Liposome/DNA complexes have been shown to be capable of mediating direct in vivo gene transfer. While in standard liposome preparations the gene transfer process is non-specific, localized in vivo uptake and expression have been reported in tumor deposits, for example, following direct in situ administration (Nabel, [1992; supra]).

If the polynucleotide encodes a sense or antisense polynucleotide or a ribozyme or DNAzyme, expression will produce the sense or antisense polynucleotide or ribozyme or DNAzyme. Thus, in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters include those described above. The expression vector may also include sequences, such as selectable markers and other sequences described herein.

Cells and animals which carry a mutant Nox4 allele or where one or both alleles are deleted can be used as model systems to study the effects of Nox4 in ROS production and/or to test for substances which have potential as inhibitory compounds. Mice, rats, rabbits, guinea pigs, hamsters, zebrafish and amphibians are particularly useful as model systems. A particularly useful insertion is a loxP sequence flanking a Nox4 gene which can be excised by cre. After a test substance is applied to the cells, the ability for ROS to be generated is determined.

The present invention provides, therefore, a mutation in or flanking a genetic locus encoding Nox4. The mutation may be an insertion, deletion, substitution or addition to the Nox4 coding sequence or its 5′ or 3′ untranslated region.

The animal model of the present invention is useful for screening for agents capable of ameliorating or mimicking the effects of Nox4. In one embodiment, the animal model produces low amounts of Nox4. Such an animal would exhibit low ROS production.

Another aspect of the present invention provides a genetically modified animal wherein said animal produces low amounts of Nox4 relative to a non-genetically modified animal of the same species. Reference to “low amounts” includes zero amounts or up to about 10% lower than normalized amounts.

Yet another aspect of the present invention provides multiple (i.e. two or more) genes which are modified. Examples of multiple genes include double Nox4 and other NADPH oxidase components.

The animal models of the present invention may be in the form of the animals including fish or may be, for example, in the form of embryos for transplantation. The embryos are preferably maintained in a frozen state and may optionally be sold with instructions for use.

The genetically modified animals may also produce larger amounts of Nox4.

Accordingly, another aspect of the present invention is directed to a genetically modified animal over-expressing genetic sequences encoding Nox4.

A genetically modified animal includes a transgenic animal, or a “knock-out” or “knock-in” animal as well as a conditional deletion mutant. Furthermore, co-suppression may be used to induce post-transcriptional gene silencing. Co-suppression includes induction of RNAi.

Two-hybrid screening is particularly useful in identifying other members of a biochemical or genetic pathway associated with Nox4. Two-hybrid screening conveniently uses Saccharomyces cerevisiae and Saccharomyces pombe. Nox4 interactions and screens for inhibitors can be carried out using the yeast two-hybrid system, which takes advantage of transcriptional factors that are composed of two physically separable, functional domains. The most commonly used is the yeast GAL4 transcriptional activator consisting of a DNA binding domain and a transcriptional activation domain. Two different cloning vectors are used to generate separate fusions of the GAL4 domains to genes encoding potential binding proteins. The fusion proteins are co-expressed, targeted to the nucleus and if interactions occur, activation of a reporter gene (e.g. lacZ) produces a detectable phenotype. In the present case, for example, S. cerevisiae is co-transformed with a library or vector expressing a cDNA GAL4 activation domain fusion and a vector expressing a Nox4-GAL4 binding domain fusion. If lacZ is used as the reporter gene, co-expression of the fusion proteins will produce a blue color. Small molecules or other candidate compounds which interact with Nox4 will result in loss of colour of the cells. This system can be used to screen for small molecules that inhibit the Nox4 function and, hence, protect the yeast against cell death and to determine the residues in Nox4 which are involved with ROS production. For example, reference may be made to the yeast two-hybrid systems as disclosed by Munder et al. (Appl. Microbiol. Biotechnol. 52(3): 311-320, 1999) and Young et al., Nat. Biotechnol. 16(10): 946-950, 1998). Molecules thus identified by this system are then re-tested in animal cells.

Antibodies directed to an extracellularly exposed portion of NADPH oxidase, such as Nox4 or a part thereof are also contemplated by the present invention. Such antibodies may be polyclonal or monoclonal antibodies but deimmunized or chimeric antibodies are particularly preferred. The antibodies may also be referred to a immunointeractive molecules and include recombinant and synthetic forms.

The present invention further provides therefore the application of biochemical techniques to render an immunointeractive molecule (eg. an antibody) derived from one animal or avian creature substantially non-immunogenic in another animal or avian creature of the same or different species. The biochemical process is referred to herein as “deimmunization”. Reference herein to “deimmunization” includes processes such as complementary determinant region (CDR) grafting, “reshaping” with respect to a framework region of an immunointeractive molecule and variable (v) region mutation, all aimed at reducing the immunogenicity of an immunointeractive molecule in a particular host (eg. a human subject). In the present case, the preferred immunointeractive molecule is an antibody such as a polyclonal or monoclonal antibody. In a most preferred embodiment, the immunointeractive molecule is a monoclonal antibody, derived from one animal or avian creature and which exhibits reduced immunogenicity in another animal or avian creature from the same or different species such as but not limited to humans.

Accordingly, one aspect of the present invention provides a variant of an immunointeractive molecule, said variant comprising a portion having specificity for an extracellularly exposed epitope on NADPH oxidase and which portion is derived from an immunointeractive molecule obtainable from one animal or avian creature wherein said variant exhibits reduced immunogenicity in another animal or avian creature from the same or different species.

As stated above, the preferred form of immunointeractive molecule is an antibody and in particular a monoclonal antibody.

Reference to “substantially non-immunogenic” includes reduced immunogenicity compared to a parent antibody, i.e. an antibody before exposure to deimmunization processes. The term “immunogenicity” includes an ability to provoke, induce or otherwise facilitate a humoral and/or T-cell mediated response in a host animal. Particularly convenient immunogenic criteria include the ability for amino acid sequences derived from a variable (v) region of an antibody to interact with MHC class II molecules thereby stimulating or facilitating a T-cell mediating response including a T-cell-assisted humoral response.

By “antibody” is meant a protein of the immunoglobulin family that is capable of combining, interacting or otherwise associating with an antigen. An antibody is, therefore, an antigen-binding molecule. An “antibody” is an example of an immunointeractive molecule and includes a polyclonal or monoclonal antibody. The preferred immunointeractive molecules of the present invention are monoclonal antibodies.

The term “antigen” is used herein in its broadest sense to refer to a substance that is capable of reacting in and/or inducing an immune response. Reference to an “antigen” includes an antigenic determinant or epitope. An extracellularly exposed portion of Nox4 is an example of a preferred antigen or epitope.

By “antigen-binding molecule” is meant any molecule that has binding affinity for a target antigen. It will be understood that this term extends to immunoglobulins (e.g. polyclonal or monoclonal antibodies), immunoglobulin fragments and non-immunoglobulin derived protein frameworks that exhibit antigen-binding activity. The terms “antibody” and “antigen-binding molecules” include deimmunized forms of these molecules.

By “antigenic determinant” or “epitope” is meant that part of an antigenic molecule against which a particular immune response is directed and includes a hapten. Typically, in an animal, antigens present several or even many antigenic determinants simultaneously. A “hapten” is a substance that can combine specificity with an antibody but cannot or only poorly induces an immune response unless bound to a carrier. A hapten typically comprises a single antigenic determinant or epitope.

As stated above, although the preferred antibodies of the present invention are deimmunized forms of murine monoclonal antibodies for use in humans, the subject invention extends to antibodies from any source and deimmunized for use in any host. Examples of animal and avian sources and hosts include humans, primates, livestock animals (e.g. sheep, cows, horses, pigs, donkeys), laboratory test animals (e.g. mice, rabbits, guinea pigs, hamsters), companion animals (e.g. dogs, cats), poultry bird (e.g. chickens, ducks, geese, turkeys) and game birds (e.g. pheasants).

Immunization and subsequent production of monoclonal antibodies can be carried out using standard protocols as for example described by Köhler and Milstein (Kohler et al., Nature 256: 495-499, 1975 and Kohler et al., Eur. J. Immunol. 6(7):511-519, 1976, Coligan et al., Current Protocols in Immunology, 1991-1997 or Toyama et al., Monoclonal Antibody, Experiment Manual, published by Kodansha Scientific, 1987). Essentially, an animal is immunized with an antigen-containing (eg. Nox4-containing sample) or fraction thereof by standard methods to produce antibody-producing cells, particularly antibody-producing somatic cells (e.g. B lymphocytes). These cells can then be removed from the immunized animal for immortalization. The antigen may need to first be associated with a carrier.

By “carrier” is meant any substance of typically high molecular weight to which a non- or poorly immunogenic substance (e.g. a hapten) is naturally or artificially linked to enhance its immunogenicity.

Immortalization of antibody-producing cells may be carried out using methods, which are well-known in the art. For example, the immortalization may be achieved by the transformation method using Epstein-Barr virus (EBV) (Kozbor et al., Methods in Enzymology 121:140, 1986). In a preferred embodiment, antibody-producing cells are immortalized using the cell fusion method (described in Coligan et al., Current Protocols in Immunology, 1991-1997), which is widely employed for the production of monoclonal antibodies. In this method, somatic antibody-producing cells with the potential to produce antibodies, particularly B cells, are fused with a myeloma cell line. These somatic cells may be derived from the lymph nodes, spleens and peripheral blood of primed animals, preferably rodent animals such as mice and rats. In the exemplary embodiment of this invention mice, spleen cells are used. It would be possible, however, to use rat, rabbit, sheep or goat cells, or cells from other animal species instead.

Specialized myeloma cell lines have been developed from lymphocytic tumors for use in hybridoma-producing fusion procedures (Kohler and Milsten supra 1976, Kozbor et al, Methods in Enzymology 121:140, 1986 and Volk et al., J. Virol. 42(1):220-227, 1982). These cell lines have been developed for at least three reasons. The first is to facilitate the selection of fused hybridomas from unfused and similarly indefinitely self-propagating myeloma cells. Usually, this is accomplished by using myelomas with enzyme deficiencies that render them incapable of growing in certain selective media that support the growth of hybridomas. The second reason arises from the inherent ability of lymphocytic tumour cells to produce their own antibodies. To eliminate the production of tumour cell antibodies by the hybridomas, myeloma cell lines incapable of producing endogenous light or heavy immunoglobulin chains are used. A third reason for selection of these cell lines is for their suitability and efficiency for fusion.

Many myeloma cell lines may be used for the production of fused cell hybrids, including, e.g. P3×63-Ag8, P3×63-AG8.653, P3/NS1-Ag-4-1 (NS-1), Sp2/0-Ag14 and S194/5.XXO.Bu.1. The P3×63-Ag8 and NS-1 cell lines have been described by Köhler and Milstein (Kohler et al., Eur. J. Immunol. 6(7):511-519, 1976). Shulman et al., Nature 276:269-270, 1978, developed the Sp2/0-Ag14 myeloma line. The S194/5.XXO.Bu.1 line was reported by Trowbridge, J. Exp. Med. 148(1):220-227, 1982.

Methods for generating hybrids of antibody-producing spleen or lymph node cells and myeloma cells usually involve mixing somatic cells with myeloma cells in a 10:1 proportion (although the proportion may vary from about 20:1 to about 1:1), respectively, in the presence of an agent or agents (chemical, viral or electrical) that promotes the fusion of cell membranes. Fusion methods have been described (Kohler et al., Nature 256:495-499, 1975, Kohler et al., Eur. J. Immunol. 6(7):511-519, 1976, Gefter et al., Somatic Cell Genet. 3:231-236, 1977 and Volk et al., J. Virol. 42(1):220-227, 1982). The fusion-promoting agents used by those investigators were Sendai virus and polyethylene glycol (EG).

Because fusion procedures produce viable hybrids at very low frequency (e.g. when spleens are used as a source of somatic cells, only one hybrid is obtained for roughly every 1×10⁵ spleen cells), it is preferable to have a means of selecting the fused cell hybrids from the remaining unfused cells, particularly the unfused myeloma cells. A means of detecting the desired antibody-producing hybridomas among other resulting fused cell hybrids is also necessary. Generally, the selection of fused cell hybrids is accomplished by culturing the cells in media that support the growth of hybridomas but prevent the growth of the unfused myeloma cells, which normally would go on dividing indefinitely. The-somatic cells used in the fusion do not maintain long-term viability in in vitro culture and hence do not pose a problem. In the example of the present invention, myeloma cells lacking hypoxanthine phosphoribosyl transferase (HPRT-negative) were used. Selection against these cells is made in hypoxanthine/aminopterin/thymidine (HAT) medium, a medium in which the fused cell hybrids survive due to the HPRT-positive genotype of the spleen cells. The use of myeloma cells with different genetic deficiencies (drug sensitivities, etc.) that can be selected against in media supporting the growth of genotypically competent hybrids is also possible.

Several weeks are required to selectively culture the fused cell hybrids. Early in this time period, it is necessary to identify those hybrids which produce the desired antibody, so that they may subsequently be cloned and propagated. Generally, around 10% of the hybrids obtained produce the desired antibody, although a range of from about 1 to about 30% is not uncommon. The detection of antibody-producing hybrids can be achieved by any one of several standard assay methods, including enzyme-linked immunoassay and radioimmunoassay techniques as, for example, described in Chou et al., U.S. Pat. No. 6,056,957.

Once the desired fused cell hybrids have been selected and cloned into individual antibody-producing cell lines, each cell line may be propagated in either of two standard ways. A suspension of the hybridoma cells can be injected into a histocompatible animal. The injected animal will then develop tumors that secrete the specific monoclonal antibody produced by the fused cell hybrid. The body fluids of the animal, such as serum or ascites fluid, can be tapped to provide monoclonal antibodies in high concentration. Alternatively, the individual cell lines may be propagated in vitro in laboratory culture vessels. The culture medium containing high concentrations of a single specific monoclonal antibody can be harvested by decantation, filtration or centrifugation, and subsequently purified.

The cell lines are tested for their specificity to detect the antigen of interest by any suitable immunodetection means. For example, cell lines can be aliquoted into a number of wells and incubated and the supernatant from each well is analyzed by enzyme-linked immunosorbent assay (ELISA), indirect fluorescent antibody technique, or the like. The cell line(s) producing a monoclonal antibody capable of recognizing the target antigen but which does not recognize non-target epitopes are identified and then directly cultured in vitro or injected into a histocompatible animal to form tumous and to produce, collect and purify the required antibodies.

Thus, the present invention provides in a first step monoclonal antibodies which specifically interact with Nox4 or an epitope thereof which is extracellularly exposed.

The monoclonal antibody is then generally subjected to deimmunization means. Such a process may take any of a number of forms including the preparation of chimeric antibodies which have the same or similar specificity as the monoclonal antibodies prepared according to the present invention. Chimeric antibodies are antibodies whose light and heavy chain genes have been constructed, typically by genetic engineering, from immunoglobulin variable and constant region genes belonging to different species. Thus, in accordance with the present invention, once a hybridoma producing the desired monoclonal antibody is obtained, techniques are used to produce interspecific monoclonal antibodies wherein the binding region of one species is combined with a non-binding region of the antibody of another species (Liu et al., Proc. Natl. Acad. Sci. USA 84:3439-3443, 1987). For example, the CDRs from a non-human (e.g. murine) monoclonal antibody can be grafted onto a human antibody, thereby “humanizing” the murine antibody (European Patent Publication No. 0 239 400, Jones et al., Nature 321:522-525, 1986, Verhoeyen et al., Science 239:1534-1536, 1988 and Richmann et al., Nature 332:323-327, 1988). In this case, the deimmunizing process is specific for humans. More particularly, the CDRs can be grafted onto a human antibody variable region with or without human constant regions. The non-human antibody providing the CDRs is typically referred to as the “donor” and the human antibody providing the framework is typically referred to as the “acceptor”. Constant regions need not be present, but if they are, they must be substantially identical to human immunoglobulin constant regions, i.e. at least about 85-90%, preferably about 95% or more identical. Hence, all parts of a humanized antibody, except possibly the CDRs, are substantially identical to corresponding parts of natural human immunoglobulin sequences. Thus, a “humanized antibody” is an antibody comprising a humanized light chain and a humanized heavy chain immunoglobulin. A donor antibody is said to be “humanized”, by the process of “humanization”, because the resultant humanized antibody is expected to bind to the same antigen as the donor antibody that provides the CDRs. Reference herein to “humanized” includes reference to an antibody deimmunized to a particular host, in this case, a human host.

It will be understood that the deimmunized antibodies may have additional conservative amino acid substitutions which have substantially no effect on antigen binding or other immunoglobulin functions.

Exemplary methods which may be employed to produce deimmunized antibodies according to the present invention are described, for example, in (Richmann et al., Nature 332:323-327, 1988, Chou et al. (U.S. Pat. No. 6,056,957), Queen et al. (U.S. Pat. No. 6,180,377), Morgan et al., (U.S. Pat. No. 6,180,377) and Chothia et al., J. Mol. Biol. 196:901, 1987).

In addition to their therapeutic value in inhibiting ROS, the antibodies may also be labelled with reporter molecules such as fluoroscent markers for use in determining the presence of NADPH oxidases with an extracellular proportion. Examples of suitable fluorescent markers include those listed in Table 5.

TABLE 5

| | | |
|---|---|---|
| Probe | Ex¹ (nm) | Em² (nm) |

Reactive and conjugated probes

| | | |
|---|---|---|
| Hydroxycoumarin | 325 | 386 |
| Aminocoumarin | 350 | 455 |
| Methoxycoumarin | 360 | 410 |
| Cascade Blue | 375; 400 | 423 |
| Lucifer Yellow | 425 | 528 |
| NBD | 466 | 539 |
| R-Phycoerythrin (PE) | 480; 565 | 578 |
| PE-Cy5 conjugates | 480; 565; 650 | 670 |
| PE-Cy7 conjugates | 480; 565; 743 | 767 |
| APC-Cy7 conjugates | 650; 755 | 767 |
| Red 613 | 480; 565 | 613 |
| Fluorescein | 495 | 519 |
| FluorX | 494 | 520 |
| BODIPY-FL | 503 | 512 |
| TRITC | 547 | 574 |
| X-Rhodamine | 570 | 576 |
| Lissamine Rhodamine B | 570 | 590 |
| PerCP | 490 | 675 |
| Texas Red | 589 | 615 |
| Allophycocyanin (APC) | 650 | 660 |
| TruRed | 490, 675 | 695 |
| Alexa Fluor 350 | 346 | 445 |
| Alexa Fluor 430 | 430 | 545 |
| Alexa Fluor 488 | 494 | 517 |
| Alexa Fluor 532 | 530 | 555 |
| Alexa Fluor 546 | 556 | 573 |
| Alexa Fluor 555 | 556 | 573 |
| Alexa Fluor 568 | 578 | 603 |
| Alexa Fluor 594 | 590 | 617 |
| Alexa Fluor 633 | 621 | 639 |
| Alexa Fluor 647 | 650 | 688 |
| Alexa Fluor 660 | 663 | 690 |
| Alexa Fluor 680 | 679 | 702 |
| Alexa Fluor 700 | 696 | 719 |
| Alexa Fluor 750 | 752 | 779 |
| Cy2 | 489 | 506 |
| Cy3 | (512); 550 | 570; (615) |
| Cy3,5 | 581 | 596; (640) |
| Cy5 | (625); 650 | 670 |
| Cy5,5 | 675 | 694 |
| Cy7 | 743 | 767 |

Nucleic acid probes

| | | |
|---|---|---|
| Hoeschst 33342 | 343 | 483 |
| DAPI | 345 | 455 |
| Hoechst 33258 | 345 | 478 |
| SYTOX Blue | 431 | 480 |
| Chromomycin A3 | 445 | 575 |
| Mithramycin | 445 | 575 |
| YOYO-1 | 491 | 509 |
| SYTOX Green | 504 | 523 |
| SYTOX Orange | 547 | 570 |
| Ethidium Bormide | 493 | 620 |
| 7-AAD | 546 | 647 |
| Acridine Orange | 503 | 530/640 |
| TOTO-1, TO-PRO-1 | 509 | 533 |
| Thiazole Orange | 510 | 530 |
| Propidium Iodide (PI) | 536 | 617 |
| TOTO-3, TO-PRO-3 | 642 | 661 |
| LDS 751 | 543; 590 | 712; 607 |

Cell function probes

| | | |
|---|---|---|
| Indo-1 | 361/330 | 490/405 |
| Fluo-3 | 506 | 526 |
| DCFH | 505 | 535 |
| DHR | 505 | 534 |
| SNARF | 548/579 | 587/635 |

Fluorescent Proteins

| | | |
|---|---|---|
| Y66F | 360 | 508 |
| Y66H | 360 | 442 |
| EBFP | 380 | 440 |
| Wild-type | 396, 475 | 50, 503 |
| GFPuv | 385 | 508 |
| ECFP | 434 | 477 |
| Y66W | 436 | 485 |
| S65A | 471 | 504 |
| S65C | 479 | 507 |
| S65L | 484 | 510 |
| S65T | 488 | 511 |
| EGFP | 489 | 508 |
| EYFP | 514 | 527 |
| DsRed | 558 | 583 |

Other probes

| | | |
|---|---|---|
| Monochlorobimane | 380 | 461 |
| Calcein | 496 | 517 |

¹Ex: Peak excitation wavelength (nm)
²Em: Peak emission wavelength (nm)

The compounds, agents, medicaments, nucleic acid molecules and other Nox4 antagonists of the present invention can be formulated in pharmaceutical compositions which are prepared according to conventional pharmaceutical compounding techniques. See, for example, Remington's Pharmaceutical Sciences, 18^{th} Ed. (1990, Mack Publishing, Company, Easton, Pa., U.S.A.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. intravenous, oral, intrathecal, epineural or parenteral.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, International Patent Publication No. WO 96/11698.

For parenteral administration, the compound may dissolved in a pharmaceutical carrier and administered as either a solution of a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered and the rate and time-course of administration will depend on the nature and severity of the condition being treated. Prescription of treatment, e.g. decisions on dosage, timing, etc. is within the responsibility of general practitioners or specialists and typically takes account of the disorder to be treated, the condition of the individual patient, the site of delivery, the method of administration and other factors known to practitioners. Examples of techniques and protocols can be found in Remington's Pharmaceutical Sciences, supra.

Alternatively, targeting therapies may be used to deliver the active agent more specifically to certain types of cell, by the use of targeting systems such as antibodies or cell specific ligands or specific nucleic acid molecules. Targeting may be desirable for a variety of reasons, e.g. if the agent is unacceptably toxic or if it would otherwise require too high a dosage or if it would not otherwise be able to enter the target cells.

Instead of administering these agents directly, they could be produced in the target cell, e.g. in a viral vector such as described above or in a cell based delivery system such as described in U.S. Pat. No. 5,550,050 and International Patent Publication Nos. WO 92/19195, WO 94/25503, WO 95/01203, WO 95/05452, WO 96/02286, WO 96/02646, WO 96/40871, WO 96/40959 and WO 97/12635. The vector could be targeted to the target cells. The cell based delivery system is designed to be implanted in a patient's body at the desired target site and contains a coding sequence for the target agent. Alternatively, the agent could be administered in a precursor form for conversion to the active form by an activating agent produced in, or targeted to, the cells to be treated. See, for example, European Patent Application No. 0 425 731A and International Patent Publication No. WO 90/07936.

In a preferred embodiment, once a subject experiences an event of the vasculature, an antagonist of vascular NADPH oxidase is immediately administered alone or in combination with other therapeutic agents such as blood clot inhibiting or dissolving agents or one or more cytokines. A pharmaceutical kit or multi-part (i.e. two or more component) pharmaceutical formulation is contemplated by the present invention for the treatment or prophylaxis of vascular disease and reperfusion injury. Such NADPH oxidase inhibitors are also useful in the treatment of cancer and to prevent ROS production in cancer cells as well as stem or progenitor cells especially during proliferation, differentiation and/or self-renewal.

The present invention further contemplates the use of a Nox4 antagonist, or inhibitor, in the manufacture of a medicament in the treatment or prophylaxis of an event or conditioning a mammalian or non-mammalian animal.

The present invention also provides the use of a benzamide and aryl sulphonates and derivative or analogs in the manufacture of a medicament for the treatment or prophylaxis of a condition or event in a mammalian or non-mammalian animal.

The present invention is further directed to the use of suramin or a derivative or analog thereof in the manufacture of a medicament for the treatment or prophylaxis of a condition or event in a mammalian or non-mammalian animal.

Although the present invention is particularly directed to Nox4, the present invention further contemplates homologs of Nox4 such as another Nox compound.

In addition, although antagonists of Nox4 or its homologs are particularly preferred, the present invention extends to agonists in cases where the promotion of ROS is desired such as to kill cancer cells.

The present invention also provides the use of tempol in the manufacture of a medicament for the treatment or prophylaxis of a condition or event in a mammalian or non-mammalian animal.

The present invention also provides the use of DPI in the manufacture of a medicament for the treatment or prophylaxis of a condition or event in a mammalian or non-mammalian animal.

The present invention is further described by the following non-limiting Examples.

The following examples investigate the role of Nox4 NADPH oxidase subunit in the development of vascular disease. Examples test the hypothesis that ROS, derived from a Nox4-containing NADPH oxidase, are major contributors to the pathogenesis of many diseases of the cardiovascular system including atherosclerosis and vascular remodeling such as restenosis, hypertension and subarachnoid hemorrhage. The Examples combine pharmacological approaches aimed at either scavenging superoxide (tempol) or specifically blocking its formation from NADPH oxidase (DPI, apocynin, suramin), with genetic strategies to directly suppress expression of the Nox4 subunit of NADPH oxidase in vivo (antisense, targeted gene-deletion). The effects of these interventions on atherogenesis is assessed using two short-term models of atherogenesis: one in rabbits (periarterial collars) and the other in mice (carotid artery ligation), as well as a longer-term model of genetic hypercholesterolemia-induced atherosclerosis in mice (apolipoprotein E-knockout mice). Also included are studies in models of subarachnoid hemorrhage (blood injection into the cisterna magna) and hypertension (angiotensin II-infusion) in rates. All of these models are widely used in the art.

### EXAMPLE 1

### Efficacy of a Superoxide Scavenging Compound and Specific NADPH Oxidase Inhibitors in Vascular Remodeling and Atherogenesis

To determine the role of NADPH oxidase-derived superoxide in atherogenesis, the effects of a superoxide scavenging compound with proven efficacy in vivo are compared with three structurally and mechanistically distinct NADPH oxidase inhibitors on ROS levels and neointima formation in rabbit and mouse models of vascular disease. The inhibitors are:

Tempol: Nitroxide molecules such as tempol (4-hydroxy-2,2,6,6-tetramethyl piperidinoxyl) have long been used as spin trapping agents for detection and quantitation of superoxide production in biological systems. However, recently these compounds have been recognized as powerful in vivo scavengers of superoxide that reduce oxidative damage in several experimental models of vascular disease including hypertension [Schnackenberg et al., Hypertension 33: 424-428, 1999; Beswick et al., Hypertension 37: 781-786, 2001], diabetes [Nassar et al., Eur. J. Pharmacol. 436: 111-118, 2002] and ischemia-reperfusion injury [Cuzzocrea et al., Shock 14: 150-160, 2000]. Since tempol acts, in part, by spin-trapping superoxide and thus obviating the formation of atherogenic downstream ROS such as H₂O₂ and OH^{●}, it is proposed herein that it should have therapeutic advantages for the treatment of atherosclerosis over conventional antioxidants such as vitamin E and flavonols.

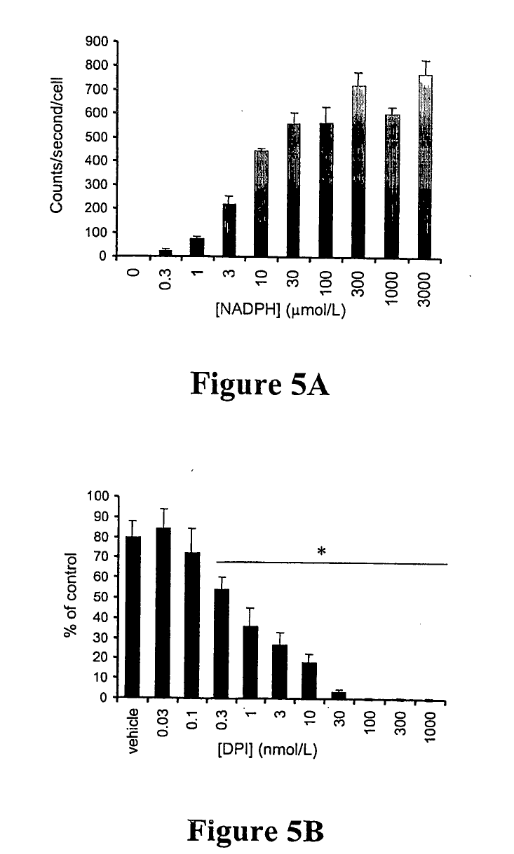

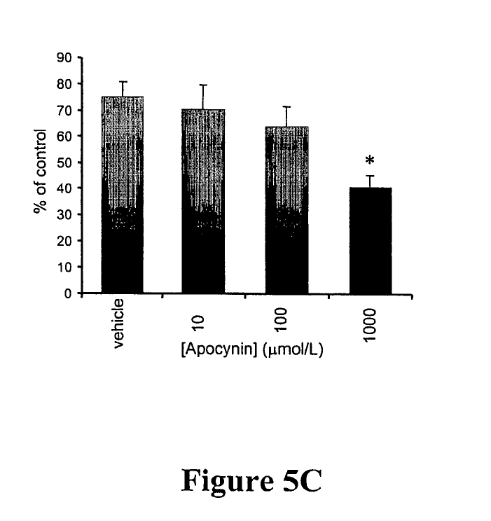

Diphenyleizeiodonium (DPI): DPI is an established flavin antagonist and inhibitor of vascular NADPH oxidase-dependent superoxide production [Paravicini et al. 2002, supra; Dusting et al., 1998, supra]. DPI is a powerful inhibitor of the Nox4-containing NADPH oxidase expressed in mouse VSMCs (FIG. 5). Importantly, the concentrations at which DPI inhibits NADPH oxidase (IC₅₀, 1 nM) are more than two orders of magnitude lower than those required to inhibit endothelial nitric oxide synthase (e.g. IC₅₀, 180-300 nM [Stuehr et al., Faseb J. 5: 98-103, 1991; Wang et al., Br. J. Pharmacol. 110: 1232-1238, 1993]). Thus, selectivity to inhibit vascular NADPH oxidase activity by local application in the periarterial collar can be achieved.

Apocynin: Apocynin is a methoxy-substituted catechol that inhibits NADPH oxidase activity by binding to its cytosolic p47phox subunit and thus preventing its association with the membrane-bound cytochrome reductase domain [Stolk et al., Am. J. Respir. Cell Mol. Biol. 11: 95-102, 1994]. It has been shown that apocynin inhibits the activity of the Nox4-containing NADPH oxidase expressed in cultured mouse VSMCs (FIG. 5). Likewise, apocynin attenuates NAD(P)H-stimulated superoxide production and increases NO bioavailability in isolated blood vessels from humans and rats [Hamilton et al., Hypertension 40: 755-762, 2002]. Importantly, administration of apocynin to DOCA-salt hypertensive rats via the drinking water has been shown to significantly reduce aortic superoxide production and blood pressure in these animals demonstrating its in vivo efficacy [Beswick et al., 2001, supra]. Whether or not apocynin inhibits vascular remodelling and atherosclerosis is assessed.

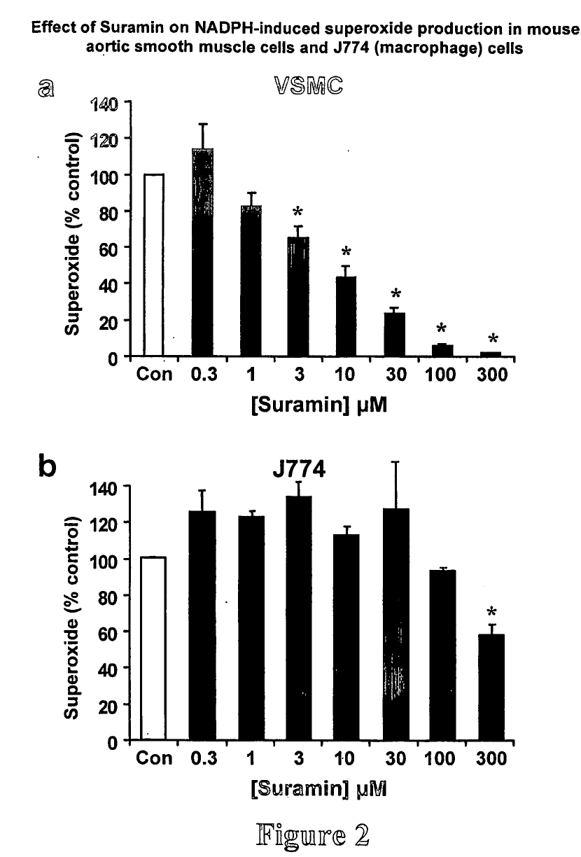

Suramin: Suramin and related sulphonated aryl compounds and/or benzamide derivates such as Reactive blue-2 and PPADS are cell-impermeable, NADPH analogs. These compounds are powerful inhibitors of the Nox4-containing NADPH oxidase in cultured mouse VSMCs (FIG. 2A). In contrast, suramin does not inhibit gp91phox-dependent NADPH oxidase activity in phagocytic cells (FIG. 2B) [Roilides et al., Antimicrob. Agents Chemother. 37: 495-500, 1993; Heyneman, Vet. Res. Commun. 11: 149-157, 1987]. Rather, these cells need to be permeabilized for suramin to exert its inhibitory effects on NADPH oxidase activity [Heyneman, 1987, supra]. It is proposed in accordance with the present invention that this selectivity derives from the fact that the NADPH binding domain of Nox4 is located extracellularly, as opposed to the intracellular location of the NADPH binding site of gp91phox. Thus, sulphonated benzamide and aryl sulphonates and derivatives or analogs represent a class of drugs which selectively inhibit vascular NADPH oxidase activity.

The experimental models are described below:

Rabbit periarterial collar: DPI is delivered periarterially to the site of injury via the collar [Gaspari et al., In: The Biology of Nitric Oxide, Part 7, Ed. S. Moncada, L. Gustafson, P. Wiklund and E. A. Higgs, Portland Press, London, pp. 72-73, 2000a] avoiding systemic side effects of DPI and ensuring that the local concentration is tightly controlled to avoid effects on eNOS. To directly compare the efficacy of all the pharmacological agents, tempol, apocynin and suramin are also delivered in this manner. In each rabbit, one artery receives either tempol (0.1 or 1 mM), DPI (10 or 100 μM), apocynin (0.1 or 1 mM) or suramin (10 or 100 μM). These concentrations are effective at inhibiting VSMC NADPH oxidase activity in vitro (FIGS. 2A and 5). The contralateral collared artery receives the appropriate vehicle to act as a within animal control and lesions will be allowed to develop over 14 days.

Mouse carotid artery ligation: 12 week-old male mice will receive either: tempol (0.1 or 1 mM in drinking water) [Beswick et al., 2001, supra], apocynin (0.15 or 1.5 mM in drinking water) [Beswick et al., 2001, supra], suramin (30 or 300 mg.kg⁻¹, i.p.) or appropriate vehicle. After one week of treatment, mice undergo ligation of one carotid artery and sham operation of the contralateral artery. Mice continue receiving appropriate treatments for a further four weeks.

ApoE^{-/-} mice: Immediately after weaning (i.e. four weeks of age), mice are assigned to receive tempol, apocynin, suramin or appropriate vehicle. Doses are those deemed most effective in the carotid artery ligation study above. All mice are maintained on a high-fat diet for six months and will continue vehicle-, apocynin- or suramin-treatment throughout this period.

Angiotensin II-induced experimental hypertension: On Day 0, rats are briefly anaesthetized (ketamine 80 mg/kg ip plus xylazine 10 mg/kg ip) and an osmotic minipump containing either saline or suramin is implanted subcutaneously. The dose rate of suramin is 300 mg/kg per 14 days. On Day 7, rats are again anaesthetized and another minipump containing saline or angiotensin II is implanted subcutaneously. The dose rate of angiotensin II is 5 mg/kg per 7 days. On Day 14, each rat is again anaesthetized and a cannula was inserted into a femoral artery for measurement of blood pressure. Angiotensin II causes a large increase in mean arterial pressure (of approx. 60-80 mmHg) in control rats.

Subarachnoid hemorrhage: On Day 0, rats are briefly anaesthetized (pentobarbital 50 mg/kg ip) and an osmotic minipump containing either saline or suramin is implanted subcutaneously. The dose rate of suramin is 300 mg/kg per 7 days. On Day 5, rats are again anaesthetized and 0.3 ml of blood is withdrawn from a femoral artery and injected into the cerebrospinal fluid around the ventral surface of the brain via the cisterna magna. In some control rats, saline is injected into the cerebrospinal fluid instead of arterial blood. The rat is allowed to recover for a further 2 days, and is then again anaesthetized on Day 7 for study.

Vascular remodeling and atherosclerosis are complex, multi-factorial processes and quantitation of their severity requires measuring multiple morphological, biochemical and molecular parameters in the blood vessel wall. The following assays are conducted:—

Vascular superoxide levels and oxidative stress: The first step in assessing the efficacy of each drug is to determine its effects on vascular superoxide levels and oxidative stress. Each compound attenuates vascular superoxide levels in all of the models above. Moreover, stoichiometric removal of superoxide (tempol) or blockade of its source (NADPH oxidase inhibitors) obviates the formation of H₂O₂ and its derivatives (HOCl⁻, OH^{●}), and, therefore, reduces overall oxidative stress in the vessel wall.

Endothelial dysfunction: This is an early clinical symptom of atherosclerosis and is manifest as a reduced capacity of arteries to dilate in response to endothelium-dependent relaxing agents (e.g. acetylcholine) (Cai and Harrison, Circ. Res. 87: 840-844, 2000]. A major cause of endothelial dysfunction is superoxide-mediated inactivation of endothelium-derived NO [Gryglewski et al., 1986, supra; Cai and Harrison, 2000, supra; Paravicini et al., 2002, supra; Dusting et al., 1998, supra]. This not only reduces the bioavailability of vasoprotective NO but also results in formation of the powerful oxidizing species peroxynitrite (ONOO⁻). Thus, by eliminating superoxide, the above interventions restore endothelial function in diseased arteries (vascular reactivity studies) and reduce peroxynitrite formation (reflected by a reduction in oxidative stress).

Inflammatory markers: Another early symptom of atherosclerosis is increased vascular expression of intercellular adhesion molecule-1 (ICAM-1), vascular cell adhesion molecule-1 (VCAM-1) and monocyte chemoattractant protein-1 (MCP-1). Up-regulation of these proteins underpins the attachment and migration of leukocytes into the subendothelial space. ROS, and particularly H₂O₂, enhances expression of ICAM-1, VCAM-1 and MCP-1 in blood vessels [Lo et al., 1993, supra]. In addition, NO normally suppresses expression of these inflammatory markers, and thus the reduced NO bioavailability in vascular disease likely contributes to their up-regulation. Restoration of the ROS/NO^{●} balance, either by scavenging superoxide or by blocking its formation, suppresses expression of inflammatory markers (real-time PCR, immunostaining) in all models of vascular disease.

VSMC proliferation: The neointimal lesions that form in the above models are largely composed of VSMCs that proliferate in the media and migrate across the internal elastic lamina (IEL). Superoxide and H₂O₂ are powerful VSMC mitogens and can stimulate migration of these cells [Griendling and Ushio-Fukai, 1998, supra]. ROS also activate matrix metalloproteinases, which degrade the IEL and facilitate the passage of VSMCs into the neointima [Belkhiri et al., Lab. Invest. 77: 533-539, 1997]. Since all of the above interventions inhibit total ROS levels (including H₂O₂) in the vascular wall, they also reduce proliferation and subsequent migration of VSMCs into the neointima [nuclear incorporation of bromodeoxyuridine (BrdU)].

Lesion size: By limiting the migration of leukocytes and VSMCs into the subendothelial space, inhibiting NADPH oxidase-derived superoxide has the overall effect of reducing lesion size in all of the above models of atherosclerosis.

Angiotensin II-induced experimental hypertension: On Day 0, rats are briefly anaesthetized ketamine 80 mg/kg ip plus xylazine 10 mg/kg ip) and an osmotic minipump containing either saline or suramin is implanted subcutaneously. The dose rate of suramin is 300 mg/kg per 14 days. On Day 7, rats are again anaesthetized and another minipump containing t saline or angiotensin II is implanted subcutaneously. The dose rate of angiotensin II is 5 mg/kg per 7 days. On Day 14, each rat is again anaesthetized and a cannula was inserted into a femoral artery for measurement of blood pressure. Angiotensin II causes a large increase in mean arterial pressure (of approx. 60-80 mmHg) in control rats.

Subarachnoid hemorrhage: On Day 0, rats are briefly anaesthetized (pentobarbital 50 mg/kg ip) and an osmotic minipump containing either saline or suramin is implanted subcutaneously. The dose rate of suramin is 300 mg/kg per 7 days. On Day 5, rats are again anaesthetized and 0.3 ml of blood is withdrawn from a femoral artery and injected into the cerebrospinal fluid around the ventral surface of the brain via the cisterna magna. In some control rats, saline is injected into the cerebrospinal fluid instead of arterial blood. The rat is allowed to recover for a further 2 days, and is then again anaesthetized on Day 7 for study.

Overall, the above measures indicate that removing excess superoxide from the artery wall reduces vascular remodelling and atherosclerosis.

### EXAMPLE 2

### Action of Antisense Against Nox4 on Superoxide Production and Neointima Development

Phosphorothioate-protected antisense oligonucleotides have previously been used in vivo to demonstrate the roles of a wide variety of genes (e.g. c-myb, c-myc, c-fos, c-jun, transforming growth factor-β, Ca²⁺-calmodulin-dependent protein kinase) in VSMC proliferation and restenosis after balloon catheter injury in rats and rabbits [Simons et al., Nature 359: 67-70, 1992; Bennett et al., J. Clin. Invest. 93:820-828, 1994; Merrilees et al., J. Vasc. Res. 31: 322-329, 1994; Villa et al., Circ. Res. 76: 505-513, 1995; Herbert et al., J. Cell Physiol. 170: 106-114, 1997]. In all of these studies, the antisense oligonucleotides are applied to the adventitial surface of the artery via pluronic gel. Importantly, adventitial application in vivo results in uniform distribution of the antisense across all layers of the blood vessel wall within 24 h [Merrilees et al., 1994, supra; Villa et al., 1995, supra]. Moreover, the antisense molecules markedly reduced expression of the target gene in injured arteries, whereas control oligonucleotide sequences (i.e. sense, non-sense) had no effect [Bennett et al., 1994, supra; Merrilees et al., 1994, supra; Herbert et al., 1997, supra].

The following experimental protocols are used:—

Mouse carotid artery ligation: 12 week-old male mice undergo surgery to ligate one carotid artery. The adventitial surface of the ligated artery will be coated with 0.25% F-127-pluronic gel containing either no additives, Nox4 antisense, or a mismatched control oligonucleotide. The effects of three antisense concentrations (0.3, 1 & 3 mg/ml are initially examined) on Nox4 mRNA expression (real-time PCR). These concentrations cover the range of antisense concentrations that have been shown previously to be effective at inhibiting gene expression in injured arteries in vivo Simons et al., 1992, supra; Bennett et al., 1994, supra; Merrilees et al., 1994, supra; Villa et al., 1995, supra; Herbert et al., 1997, supra]. The antisense concentration deemed to be most effective at inhibiting Nox4 expression will be used for all subsequent studies. After four weeks, mice are sacrificed and their ligated and sham-operated carotid arteries removed for the endpoint measurements.

Rabbit periarterial collar: The effects of Nox4 antisense on superoxide production and lesion development induced by periarterial collars in rabbits is examined. In these studies, the periarterial collar acts both as a stimulus to induce neointimal lesions, and as a vehicle for direct local application of antisense to the adventitial surface of the artery. To obtain an antisense that is effective against rabbit Nox4, a similar design strategy to that which is used in mouse VSMCs is used. Nox4 antisense is delivered to the left artery for 14 days, while the contra-lateral artery will receive vehicle (saline or Oligofectamine see below). Antisense molecules are established as being distributed across all layers of the artery wall and localized within the cytosolic and nuclear compartments of the vascular cells. This is achieved by fluorescence imaging of collared artery sections after in vivo treatment with FITC-labeled antisense molecules, as we have done previously in cultured VSMCs. If it is deemed that antisense molecules are not adequately taken up into cells, they are complexed to a transfection reagent (Oligofectamine) before being loaded into the mini-pump. Oligofectamine facilitates entry of Nox4 antisense into the cytosolic and nuclear compartments of mouse VSMCs. Optimization experiments are then performed to evaluate the antisense concentration required to inhibit Nox4 expression in collared arteries. Initially, the effects of three antisense concentrations (0.3 μM, 1 μM and 3 μM) are examined covering the range of concentrations shown to be effective at inhibiting gene expression in vitro [Drummond, In: Australian Health and Medical Research Congress, Melbourne, Australia, pp 335, 2002; Bengsston et al., In: Australian Health and Medical Research Congress, Melbourne, Australia, p 1203, 2002]. Nox4 mRNA expression in the antisense-treated collared arteries is compared to that in the upstream, non-collared segment of artery, as well as to the contra-lateral, vehicle-treated collared artery. The effect of Nox4 inhibition is then determined on the endpoint measurements. To ensure that any effects of oligonucleotide treatment are antisense specific, identical studies are performed in rabbits in which one collared artery is treated with a scrambled oligonucleotide.

### EXAMPLE 3

### Effects of Targeted Nox4 Gene Deletion Versus gp91phox Gene Deletion on Vascular Remodelling and Atherosclerosis in Mice

Targeted deletion of p47phox reduces atherosclerotic lesion area in the descending aorta of hypercholesterolemic ApoE^{-/-} mice [Barry-Lane et al., 2001, supra]. Since p47phox is an essential subunit of both the vascular and phagocytic isoforms of NADPH oxidase, it is unclear which of these enzymes (and which Nox subunit) is important in the development of atherosclerosis in mice. Therefore, the effects of targeted Nox4 gene deletion are compared with gp91phox gene deletion on atherogenesis in both the carotid artery ligation and ApoE-knockout models of atherosclerosis in mice.

The following mouse modes are used:—

gp91phox^{-/-}. The F1 generation of these mice were initially created by targeted deletion of the gp91phox gene in embryonic stem cells of 129/SvJ×C57BL/6 mice followed by homologous recombination [Pollock et al., Nat. Genet. 9: 202-209, 1995]. The mice were then backcrossed to the wild-type C57BL/6 strain for 10 generations [Pollock et al., 1995, supra].

Nox4^{-/-}: Homozygous Nox4-deficient mice (Nox4^{-/-}) are created commercially (Ozgene, Wash.) using a similar protocol to that used for gp91phox gene deletion [Pollock et al., 1995, supra]. Given that p47phox knockout mice, who display no NADPH oxidase activity, are viable [Barry-Lane et al., 2001, supra], disruption of the Nox4 gene should not affect embryo survival.

The following experimental protocols are used:—

Mouse carotid artery ligation: Carotid artery ligation is performed on age-matched wild-type, Nox4^{-/-} and gp91phox^{-/-} mice. After four weeks, mice are sacrificed and their ligated and sham-operated carotid arteries removed for measurements of Nox4 and gp91phox mRNA (real-time PCR) and protein (Western blot assay) expression. These studies are essential as they not only confirm that the appropriate gene has been silenced but will also provide information on whether gene deletion causes compensatory increases in the expression of other NADPH oxidase subunits. Finally, superoxide production will be measured in peritoneal macrophages isolated from each strain to determine the effect of gene deletion on phagocytic NADPH oxidase activity.

Nox4 knock out mice and carotid artery ligation are also used to confirm that any beneficial effects of suramin on vascular remodeling are truly the result of selective inhibition of the vascular Nox4-containing NADPH oxidase. In these studies, Nox4^{-/-} mice are treated with the dose of suramin found to be most effective in the studies. If suramin acts by inhibiting Nox4, no further effects of this drug on superoxide production, endothelial function and other endpoint measurements should be observed over that already seen in the Nox4^{-/-} mice.

ApoE^{-/-} mice: Nox4^{-/-} and gp91phox^{-/-} mice are crossed with homozygous ApoE^{-/-} mice to create two double-knock out strains (i.e. ApoE^{-/-}/Nox4^{-/-}& ApoE⁴/gp91phox^{-/-}). For these experiments, uncrossed ApoE^{-/-} mice serve as the control group. An extra group of ApoE^{-/-}/Nox4^{-/-} mice are included which are treated with suramin to confirm that any beneficial effects of this drug on atherogenesis are the result of selective inhibition of Nox4. Mice from each strain are fed a high-fat diet for six months after which time they will be sacrificed and their aortas removed for measurements of Nox4 and gp91phox mRNA (real-time PCR) and protein (Western blot assay) expression.

Vascular superoxide production is expected to be reduced in mice lacking the Nox4 gene. In contrast, deletion of gp91phox should have no effect on vascular superoxide production but will suppress phagocytic NADPH oxidase activity. Therefore, Nox4-deficient animals, but not gp91phox-deficient animals, will display lower levels of vascular oxidant stress leading to reduced vascular remodeling and atherosclerosis.

### EXAMPLE 4

### Role of Nox4 in Superoxide Production

This example investigates the role of the Nox4 subunit in NADPH oxidase-dependent superoxide production in unstimulated mouse VSMCs.

Mice

Thirteen week-old, male C57BL6/J mice, purchased from the Animal Resource Centre (Australia) and maintained on a normal chow diet, were used. For all experiments, mice were heparinized (250 IU, i.p.) and anaesthetized with Isoflo inhalation anaesthetic (Abbot), prior to being killed by decapitation.

VSMC Culture

For each culture, thoracic aortas from two mice were isolated and cleared of adhering fat and connective tissue, before being placed in digestion medium (i.e. DMEM containing 0.5 mg/ml elastase, 1.0 mg/ml collagenase and 1.25 mg/ml trypsin) and incubated at 37° C. for 5 mins. The adventitial layer of the blood vessels were then peeled off with fine forceps and digestion medium was flushed through the vessel lumen to dislodge endothelial cells. The remaining tube of medial smooth muscle cells was then cut into ring segments (2-3 mm) and transferred to a microcentrifuge tube containing 500 μL of digestion medium. After incubating for 90 mins at 37° C., VSMCs were dispersed by pipetting up and down with a P1000 pipette tip and plated onto a 60 mm culture dish containing 5 mL DMEM supplemented with 10% v/v heat inactivated foetal bovine serum (FBS, CSL), 2 mmol/L L-glutamine (CSL), 50 U/ml penicillin and 50 μg/ml streptomycin (CSL). The cells were maintained at 37° C. in a 5% v/v CO₂ humidified incubator and passaged in a 1:4 ratio weekly. Cells between passages 4 and 20 were used for experiments.

Antisense Design and Synthesis

Six antisense sequences were designed with the aid of Gene Runner Software (Hastings Software, Inc.) to complement various sites around the translation start codon of the native mouse Nox4 mRNA (GenBank Accession No. NM_{—}015760) (Table 6). For one of these antisense molecules, +13/+33, scrambled and mismatch control sequences were also designed. The scrambled sequence contained the same base composition as the antisense but in a random order, while the mismatch sequence differed from the antisense sequence in three base positions (Table 6). All phosphorothioated oligonucleotides were commercially synthesized and polyacrylamide gel purified (Sigma Genosys).

TABLE 6
Primer and probe sequences and concentrations for real-time PCR

| Gene | Primer sequence | Probe sequence | cDNAco |
|---|---|---|---|
| (NCBI Acc.) | (concentration in nmol/L) | (concentration in nmol/L) | nc. |
| Nox4 | Fwd: 5-TGTTGGGCCTAGGATTGTGTT | 5′-FAM-AAGCAGAGCATCTGCATCTGTCCTCAACC | 20 ng |
| (NM_015760) | (150) [SEQ ID NO:9] | (200) [SEQ ID NO:10] | |
| | Rev: 5′-AAAAGGATGAGGCTGCAGTTG | | |
| | (300) [SEQ ID NO:11] | | |
| Nox1 | Fwd: 5′-TGGTCATGCAGCATTAAACTTTG | Not applicable | 20 ng |
| | (300) [SEQ ID NO:12] | | |
| | Rev: 5′-CATTGTCCCACATTGGTCTCC | | |
| | (300) [SEQ ID NO:13] | | |
| 18s | Fwd: 5′-CGGCTACCACATCCAAGGAA | 5′-VIC-TGCTGGCACCAGACTTGCCCTC-TAMRA | |
| | (40) [SEQ ID NO:14] | (200) [SEQ ID NO:15] | |
| | Rev: 5′-GCTGGAATTACCGCGGCT | | |
| | (80) [SEQ ID NO:16] | | |

Antisense Transfection

Mouse VSMCs were plated sparsely onto 96-well ViewPlates (Packard Bioscience) (for superoxide measurements) or onto 35 mm culture dishes (for RNA extraction) such that they were 30-50% confluent at the time of transfection 24 h later. At the time of transfection, cells were washed with serum- and antibiotic free DMEM and were then incubated in serum- and antibiotic-free DMEM containing 8 μL/mL Oligofectamine Transfection Reagent (Invitrogen Life Technologies) complexed with the antisense (0-1000 nmol/L), mismatch (500 nmol/L) or scrambled (560 nmol/L) oligonucleotides. After 4 h incubation at 37° C., an equal volume of DMEM containing 10% v/v FBS was added (i.e. to give a final FBS concentration of 5% v/v) and the cells were incubated at 37° C. for 24 h before RNA extraction or for up to 72 h before being assayed for superoxide production.

Superoxide Measurements

Superoxide production in mouse VSMCs was assessed by lucigenin-enhanced chemiluminescence. To examine the effects of pharmacological agents on superoxide production, VSMCs were plated onto the wells of a 96-well ViewPlate and allowed to grow to confluence. Twenty-four h prior to assaying for superoxide, the regular cell culture media was exchanged for DMEM containing a reduced FBS concentration (5% v/v) along with L-glutamine and antibiotics. In some experiments, this media was further supplemented with the NADPH oxidase inhibitor, apocynin (10-1000 μmol/L), or vehicle (DMSO 0.1%). The following day, the cell culture media was exchanged for a Krebs-Hepes pre-incubation solution containing DETCA (3 mmol/L, inhibitor of Cu²⁺/Zn²⁺-superoxide dismutase) and one or more of the following drugs: NADPH (3-3000 μmol/L; substrate for NADPH oxidase); apocynin (10-1000 μmol/L); DPI (0.03-1000 nmol/L; inhibitor of flavoenzymes). After 45 minutes pre-incubation at 37° C. the pre-incubation solutions were replaced with 200 μL of a Krebs-HEPES assay solution containing lucigenin (5 μmol/L) and the appropriate drug treatment(s). Average photon emission per second per well was monitored over a 20-min period in a TopCount single photon counter (Packard Bioscience).

Cell Viability

After measuring superoxide production in VSMCs, cells were washed with 250 μL of Krebs-Hepes and incubated for 3 h in 100 μL of 20% CellTiter 96 (registered trademark) AQᵤₑₒᵤₛ One Solution Cell Proliferation Assay (Promega) dissolved in Krebs-Hepes as per the manufacturer's instructions. Cell viability was assessed by measuring the absorbance of the supernatant at 490 nm.

RNA Extraction

RNA was extracted from cultured and freshly isolated mouse VSMCs, and from freshly isolated whole aortas using RNAwiz (Ambion) according to the manufacturer's protocol. RNA concentrations were determined spectrophotometrically by measuring absorbance at 260 nm.

Reverse Transcription (RT) Reaction

RNA (100-500 ng) was reverse transcribed using TaqMan Reverse Transcription Reagents (PE applied Biosystems) according to the manufacturer's protocol. As a control for genomic DNA contamination in subsequent real-time PCR, parallel RT reaction mixtures containing all reagents except the Reverse Transcriptase were prepared for all RNA samples.

Real-Time PCR

Real-time PCR and the ΔΔCt method were used as previously described to examine mRNA expression of Nox4 and Nox1 relative to a “reference” sample. [Paravicini et al., 2002, supra; Winer et al., 1999, supra]. Primers and a 5′-FAM-labeled fluorescent probe for Nox4 were designed using Primer Express software (PE Biosystems) and the published sequence for the mouse homolog of the Nox4 gene (Table 7). For Nox1, where a mouse sequence has not been described, a region of high homology between the human and rat homologues of the gene was identified and used to design primers, again with Primer Express (Table 7). Since probe binding in real-time PCR will not tolerate a single base mismatch, SYBR (registered trademark) Green (PE Biosystems) was used in the Nox1 PCR mixture in place of a labeled probe. 18S ribosomal RNA was used as the internal standard for each reaction and was detected with commercially available rodent 18S primers and a 5′-VIC-labeled probe (PE Biosystems; Table 7).

Nox4 was amplified in duplex with 18S in PCR mixtures (25 μL final volume) containing 1× TaqMan (registered trademark). Universal PCR master-mix (PE Biosystems), cDNA template (5 ng) and optimized primer and probe concentrations for 18S and Nox4 (Table 7). The PCR mixture for Nox1 contained 1×SYBR (registered trademark). Green master-mix (PE Biosystems), cDNA template (20 ng) and optimized primer concentrations, in a final volume of 25 μL. PCR thermal cycle parameters were 2 min at 50° C., 10 min at 95° C. and 40 cycles of 95° C. for 30 s and 60° C. for 1 min. Reactions were performed and fluorescence monitored in the ABI Prism 7700 Sequence Detector (PE Biosystems).

TABLE 7

Nox4 antisense, mismatch and scrambled
oligonucleotide sequences

| | |
|---|---|
| Nox4 | Oligonucleotide sequence |

| | |
|---|---|
| +13/+33 antisense | TTGGCCAGCCAGCTCCTCCA |
| | [SEQ ID NO:17] |
| +13/+33 mismatch | TAGGCCAGCAAGCTCCTACA |
| | [SEQ ID NO:18] |
| +13/+33 scrambled | CGTCACGCTCAGCTCACCGT |
| | [SEQ ID NO:19] |

Statistical Analysis

Results are expressed as mean±standard error of the mean (SEM) of n independent experiments. Superoxide production is expressed as counts per second per well, normalized to cell viability and as a percentage of untreated or vehicle-treated control. The amount of mRNA is expressed as a fold-change relative to the “reference” sample. Statistical analyses were carried out by one-way repeated measures ANOVA followed by Tukey all pairwise multiple comparison procedures. Differences were considered statistically significant at P<0.05.

NADPH Oxidase Activity in Mouse VSMCs

Superoxide generation was barely detectable in unstimulated mouse cultured VSMCs.

However, incubation of these cells with NADPH, the preferred electron substrate for NADPH oxidase, caused a concentration-dependent increase in superoxide production (EC₅₀, 5.0±0.6 μmol/L; FIG. 5A). NADPH-driven superoxide production was inhibited in a concentration-dependent manner by the flavin antagonist and reputed NADPH oxidase inhibitor, diphenylene iodonium (IC₅₀, 1±0.4 nmol/L; FIG. 5B). A structurally unrelated inhibitor of NADPH oxidase, apocynin, had no effect on NADPH-driven superoxide production after 45 mins, but inhibited this response by ˜50% after 24 h (FIG. 5C). Collectively, these data provide strong functional evidence for the presence of NADPH oxidase in cultured mouse VSMCs.

Nox4 is Expressed in Mouse VSMCs

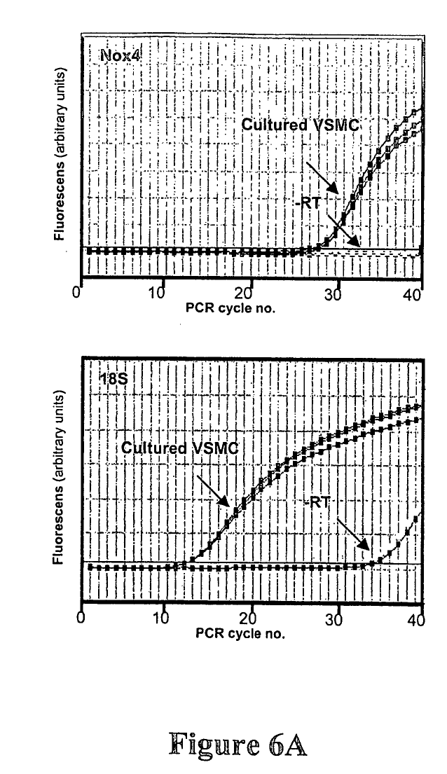

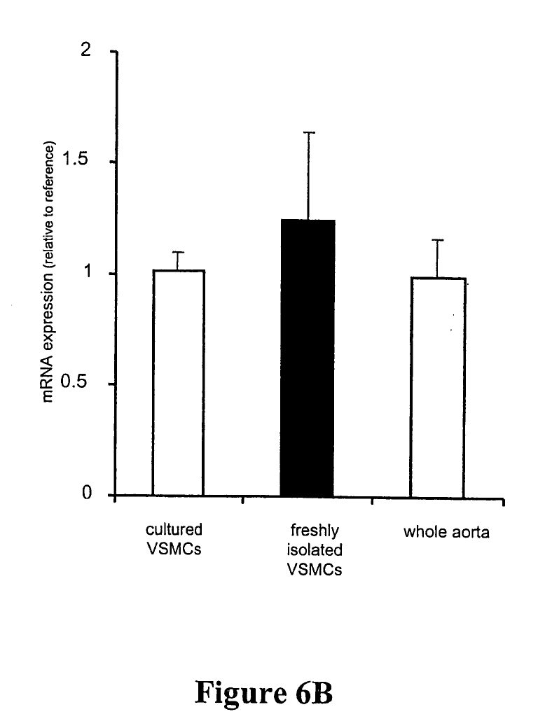

Having established that NADPH oxidase is present in cultured mouse VSMCs, the expression of Nox4 was examined in these cells using real-time RT-PCR. Nox4 mRNA appeared to be highly expressed in RNA extracts from cultured mouse VSMCs (ΔCt=12.3±0.2; FIG. 6A). Importantly, this level of Nox4 expression relative to 18S was similar to that observed in both whole aortas (ΔCt=12.2±0.5) and in VSMCs (ΔCt=12.4±0.3) freshly isolated from healthy, 13 week-old mice (FIG. 6B). In contrast to Nox4, Nox1 could not be detected in either cultured VSMCs or in freshly isolated VSMCs and whole aortas. Note that this latter negative finding was not due to ineffective primers since expression of Nox1 was readily detectable in RNA obtained from mouse colon, a tissue known to express high levels of Nox1.

Nox4 Antisense Inhibits NADPH-Driven Superoxide Production

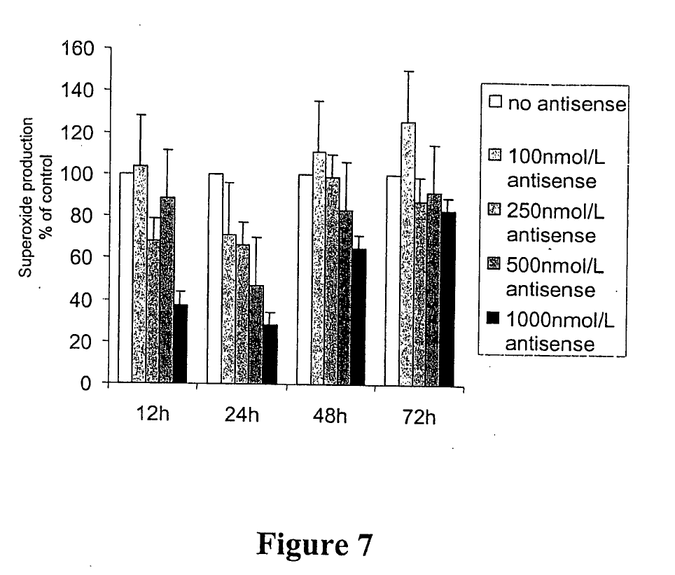

To directly assess the role of Nox4 in NADPH oxidase activity in mouse VSMCs, an antisense approach was employed. Six phosphorothioate antisense molecules designed to bind to various sites around the translation start codon of the native Nox4 mRNA were tested. Of the six antisense molecules screened, one sequence, +13/+33, caused a significant 45% reduction in NADPH-driven superoxide production (n—4). Further characterization of this effect demonstrated that the +13/+33 antisense caused both a time- and concentration-dependent inhibition of NADPH-driven superoxide production (FIG. 7). Although some inhibition was observed after 12 h, the greatest effect was seen after 24 h and with an antisense concentration of 1000 nM. By 48 h, the degree of inhibition at all antisense concentrations was markedly attenuated, while at 72 h it had completely disappeared.

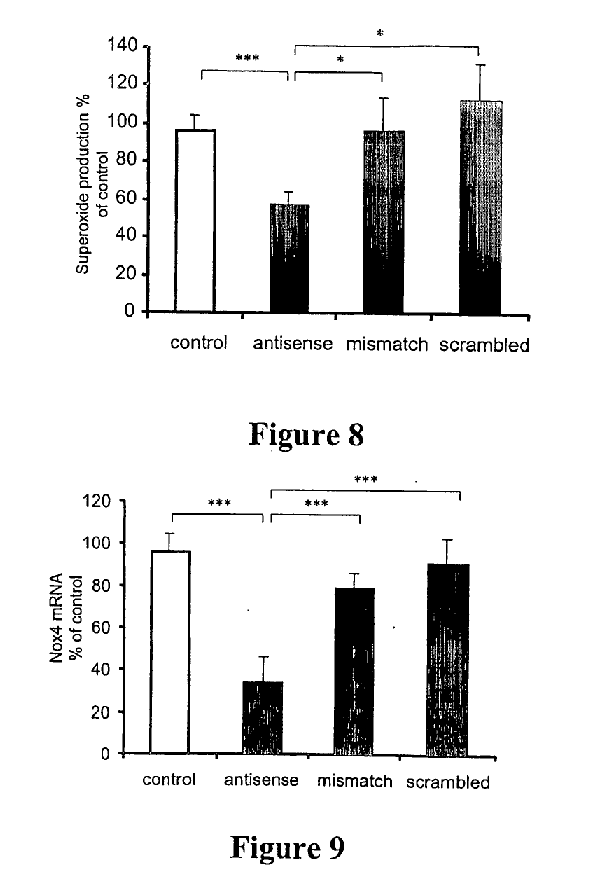

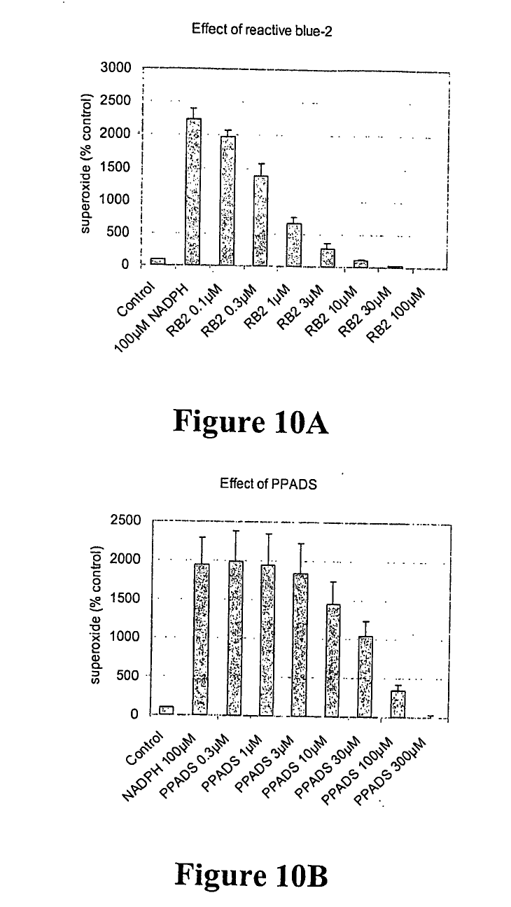

To exclude the possibility of a non-specific action of the +13/+33 antisense in the above studies, in a second series of experiments, its effect on NADPH-driven superoxide production with those of mismatch and scrambled oligonucleotide sequences was compared. While the antisense again caused a significant 41% reduction in NADPH-driven superoxide production, neither the mismatch nor the scrambled sequence had any effect (FIG. 8).

Downregulation of Nox4 in mRNA by Antisense

To establish if the inhibition of NADPH-driven superoxide production observed after Nox4 antisense treatment was reflected at the molecular level, real-time PCR was used to examine the effects of antisense on Nox4 mRNA expression. Cells incubated with antisense for 24 h displayed a 65% reduction in Nox4 mRNA expression whereas no effect was observed in cells transfected with mismatch or scrambled sequences (FIG. 9). Also, Nox4 antisense did not appear to cause a compensatory increase in mRNA expression of Nox1.

This Example provides direct evidence that Nox4 is a critical component of the superoxide generating NADPH oxidase complex in VSMCs. Nox4 was found to be expressed at high levels in both freshly isolated and cultured mouse VSMCs and down-regulation of Nox4 mRNA expression with sequence specific antisense markedly attenuated NADPH oxidase activity in these cells.

### EXAMPLE 5

### Animal Models and Methods

Rabbit peri-arterial collar model: This rabbit model of artery disease over 12 years ago [Dusting et al., J. Cardiovasc. Pharmacol. 16: 667-674, 1990; Arthur et al., J. Vasc. Res. 31: 187-194, 1994; Dusting et al., American Journal of Cardiology 76: 24E-27E, 1995; Arthur et al., Arteriosclerosis, Thrombosis and Vascular Biology 17: 737-740, 1997; Yin and Dusting, Clinical and Experimental Pharmacology and Physiology 24: 436-438, 1997; Yin et al., Journal of Vascular Research 35: 156-164, 1998; Gaspari et al., 2000a, supra; Gaspari et al., Clinical and Experimental Pharmacology and Physiology 27: 653-655, 2000b; Paravicini et al., 2002, supra]. Neointimal thickening develops after hollow, silastic collars are placed around the common carotid arteries. The advantages of this model are (1) lesion formation occurs within days, and (2) drugs can be administered locally to the site of vascular injury without systemic actions. The neointimal lesions display many characteristics of early-stage human atheroma including up-regulation of endothelial ICAM-1, VCAM-1 and MCP-1 expression within 48 h, followed by infiltration of leukocytes, accumulation of cholesterol esters, and deposition of collagen and fibronectin [Kock et al., Arterioscler. Thromb. 12: 1447-1457, 1992; Kockx et al., Arterioscler. Thromb. 13: 1874-1884, 1993]. The neointima contains mainly VSMCs that replicate in the media before migrating across the IEL [Kockx et al., 1993, supra]. In addition, collared arteries undergo functional changes reminiscent of human atheroma including hypersensitivity to the constrictor action of 5-hydroxytryptamine [Dusting et al., 1990, supra; Kockx et al., 1992, supra] and impaired relaxation to acetylcholine [Dusting et al., 1990, supra; Arthur et al., 1994, supra; De Meyer et al., J. Cardiovasc. Pharmacol. 29(12): S205-207, 1992; Yin et al., 1998, supra; Gaspari et al., 2000a, supra; Gaspari et al., 2000b, supra; Paravicini et al., 2002, supra]. Importantly, the inventors have shown that NADPH oxidase activity is increased in collared arteries and that this contributes to endothelial dysfunction.

Mouse carotid artery ligation: This is a widely used mouse model of arterial remodeling whereby neointimal lesions are induced over a short time period (weeks) by complete ligation of the carotid artery just proximal to its bifurcation [Kumar and Linder, Arterioscler. Thromb. Vasc. Biol. 17: 2238-2244, 1997]. Also, being a mouse model, it is amenable to studies aimed at determining the roles of specific genes in atherogenesis. Cessation of blood flow by ligation of one of the common carotid arteries results in VSMC proliferation and the formation of a VSMC-rich neointima proximal to the ligature [Kumar and Linder, 1997, supra]. Early local inflammation is evident with increased expression of adhesion molecules and accumulation of leukocytes throughout the vessel wall [McPherson et al., Arterioscler. Thromb. Vasc. Biol. 21: 791-796, 2001]. While it is well established that the endothelium remains intact throughout lesion development [Kumar and Linder, 1997, supra], no studies have examined whether endothelial function or NADPH oxidase activity are altered in this model.

Apolipoprotein E-deficient mice: NADPH oxidase is a major source of excess superoxide production in atherosclerotic vessels [Drummond et al., 2001, supra; Jiang et al., European Journal of Pharmacology 424: 141-149, 2001. Moreover, this contributes to failure of endothelium-dependent vasorelaxation in the aorta, even when there are minimal fatty lesions [Jiang et al., 2001, supra]. Lesions develop in the aortic arch, branch points of the carotid, subclavian, mesenteric, renal and iliac arteries, and in the coronary and pulmonary arteries from five weeks of age when monocytes attach to the endothelium and migrate into the subendothelial space [Breslow, Science 272: 685-688, 1996]. At 10-15 weeks, fatty streaks appear consisting of foam cells and VSMCs that divide in the medial layer before migrating across the IEL into the neointima [Breslow, 1996, supra]. By 20 weeks, lesions are similar to human fibrous plaques consisting of a necrotic core and a fibrous cap of VSMCs surrounded by elastic fibres and collagen [Breslow, 1996, supra]. The major advantages of this model are that (1) most stages of human atherosclerosis are represented, (2) atherosclerosis can be accelerated by a high fat diet, and (3) aortic segments display endothelial dysfunction in which excess NADPH oxidase-derived superoxide plays a causative role.

Luminescence detection of vascular superoxide and ROS: Lucigenin- and luminol-enhanced chemiluminescence is used as quantitative measures of vascular superoxide production and general oxidant stress, respectively, as previously described [Paravicini et al., 2002, supra; Dusting et al., 1998, supra]. Lucigenin is a validated technique for detecting superoxide in vascular tissues [Skatchkov et al., Biochem. Biophys. Res. Commun. 254: 319-324, 1999]. Moreover, using nitroblue-tetrazolium, superoxide signal generated from VSMCs exposed to 5 μM lucigenin is no higher than that in unexposed cells. Likewise, luminol has been used previously to measure vascular peroxynitrite and H₂O₂ formation [Laursen et al., Circulation 103: 1282-1288, 2001] and is thus a convenient indicator of vascular oxidant stress per se. Ring segments of artery for use in lucigenin assays are pre-incubated for 45 mins in diethyldithiocarbamate (DETCA; 3 mM) to inactivate endogenous Cu²⁺/Zn²⁺ superoxide dismutase. Some of these DETCA-treated arteries are further pre-incubated with NADPH (100 μM) to ensure adequate substrate availability for NADPH oxidase. Artery segments will then transferred to separate wells of an Opaque 96-well plate containing either lucigenin (5 μM) or luminol (100 μM), as well as the appropriate substrate treatment. Photon emission per second from each well will be measured using a Single Photon Counter and normalised to dry tissue weight to account for differences in blood vessel sizes.

Fluorescence detection of vascular superoxide and ROS: Dihydroethidium (DHE) and reduced 2′-7′-dichlorofluorescein diacetate (DCFH-DA) is used to confirm luminescence results and to localise vascular superoxide production and oxidant stress, respectively, as have previously described [Paravicini et al., 2002, supra; Dusting et al., 1998, supra; Tarpey and Fridovich, Cir. Res. 89: 224-236, 2001]. Blood vessel segments (carotid and aorta) are frozen in OCT, cut into 20 μm sections and mounted on gelatin-coated slides. Sections are treated with 10 μl of DHE (2 μM) or DCFH-DA (5), prior to coverslipping and incubating in the dark at 37° C. for 45 mins. Sections are then excited (568 nm for DHE; 498 nm for DCFH-DA) and the emitted light (585 nm for DHE; 522 nm for DCFH-DA) visualized and imaged using a confocal microscope.

Phagocytic NADPH oxidase activity: Mice are given an intraperitoneal injection of thioglycollate (1 mL of 4% w/v solution) 24 h prior to sacrifice, to recruit macrophages to the abdominal cavity. At the time of sacrifice, these cells are harvested by lavage and phorbol ester-stimulated superoxide production (i.e. phagocytic NADPH oxidase activity) measured by lucigenin-enhanced chemiluminescence (lucigenin) [Kirk et al., Arterioscler. Thromb. Vasc. Biol. 20: 1529-1535, 2000].

Real-time PCR measurement of mRNA expression: Real-time PCR and the ΔΔCt method is used to measure mRNA expression in vascular tissues (carotid artery and aorta) as previously described [Paravicini et al., 2002, supra; Dusting et al., 1998, supra; Winer et al., Anal. Biochem. 270: 41-49, 1999]. Primers and 5′-FAM labelled fluorescent probes for Nox4, gp91phox, ICAM-1, VCAM-1 and MCP-1 are designed with Primer Express Software from the published mRNA sequences for the rabbit and mouse homologs of each gene. 18s rRNA is used as an internal standard for each reaction using commercially available rodent 18s primers and a 5′-VIC-labeled probe. Nox4, gp91phox, ICAM-1, VCAM-1 and MCP-1 is each amplified in duplex with 18s in PCR mixtures containing Taqman Universal PCR master mix, cDNA template and optimised concentrations of primers and probes. Real-time PCR will be performed and fluorescence monitored in the ABI Prism 7700 Sequence Detector.

Western blot assays: This is used to quantify protein expression of Nox4, gp91phox, ICAM-1, VCAM-1 and MCP-1 in rabbit and mouse arteries as previously described [Sun et al., European Journal of Pharmacology 320: 29-35, 1997] using primary antibodies against each of the proteins (all publically available), secondary antibodies conjugated to horseradish peroxide and the ECL detection system.

Immunostaining: Localization and expression of ICAM-1, VCAM-1 and MCP-1 is examined by immunostaining fresh frozen sections of artery with mouse monoclonal anti-ICAM-1 (1:200 dilution), anti-VCAM-1 (1:200 dilution) and anti-MCP-1 (1:50 dilution) antibodies, respectively. A biotinylated goat anti-mouse secondary antibody (SantaCruz) and streptavidin-horseradish peroxide with 3,3′-diaminobenzamine as the color substrate, is used for all staining runs.

VSMC proliferation: Animals receive two subcutaneous injections of BrdU (rabbits 30 mg.kg⁻¹ i.p.; mice 0.1 mg.kg⁻¹ s.c.) 24 h and 6 h before euthanasia [Kumar and Lindner, 1997, supra]. Arteries are removed, snap frozen in OCT and cut into 4 μm sections for mounting on gelatin-coated slides. The extent of VSMC proliferation in the media and intima is determined by staining vessels with a mouse monoclonal antibody against BrdU (1:200 dilution) [Kumar and Lindner, 1997, supra]. The numbers of total and stained nuclei are counted separately in the media and intima to allow calculation of the BrdU labelling indices [i.e. (stained nuclei/total nuclei)×100] for each layer.

Lesion size was quantitated as follows:

Rabbit carotid artery: A 1 mm ring segment is isolated from the centre of all collared arteries, then fixed and slide-mounted to allow quantitation of neointima formation [expressed as an intima:media ratio (IMR)] as previously described [Dusting et al., 1990, supra; Arthur et al., 1994, supra; Dusting et al., 1995, supra; Arthur et al., 1997, supra; Yin and Dusting, 1997, supra; Yin et al., 1998, supra; Gaspari et al., 2000a, supra; Gaspari et al., 2000b, supra; Paravicini et al., 2002, supra].

Mouse carotid artery ligation: After euthanasia, mice are perfusion fixed with 4% v/v paraformaldehyde. Ligated and sham-operated carotid arteries are excised, immersion fixed in ethanol and embedded in the same paraffin block. The ligated carotid artery is cut into 4 μm sections starting from the ligature towards the aortic arch. A standardized reference point is set at the location where the ligature does not distort the vessel and where the elastic laminae remain intact (i.e. between 0.05 mm and 0.13 mm from the ligature). The IMRs of cross sections at 0.2, 0.3 and 0.4 mm from the reference point are measured using the MCID.

ApoE^{-/-} mice: Whole aortas are isolated and cut open via an incision along the ventral wall [Jiang et al., 2001, supra]. Tissues are rinsed with 60% v/v isopropanol and stained with oil red O (0.5%) for 10 minutes at room temperature. After staining, tissues are rinsed in 60% v/v sopropanol and preserved in 10% v/v neutral buffered formalin. The en face surface of the aorta is then imaged and lesion area (red-stained) quantified using MCID imaging analysis software.

In vitro vascular reactivity studies: This is used to quantify NO bioavailability in aortic and carotid artery ring segments (˜3 mm) as previously described [Yin et al., 1998, supra; Gaspari et al., 2000a, supra; Gaspari et al., 2000b, supra; Paravicini et al., 2002, supra; Drummond et al., Br. J. Pharmacol. 129: 811-819, 2000]. Rabbit carotid artery rings are set up in conventional organ baths, suspended by two stainless steel wire hooks (250 μm), one connected to an isometric force transducer and the other to a micrometer-adjustable support. Mouse carotid artery and aorta segments are suspended in a Mulvany-Halpern style myograph using 40 μm stainless steel wires. All vessels are equilibrated for 20 minutes, tensioned to an optimal resting diameter, and maximally contracted with an isotonic 125 mM K⁺ solution (KPSSₘₐₓ). To eliminate the potential contribution of prostacyclin and EDHF to vasorelaxation responses (i.e. to isolate NO-mediated responses) all rings are treated with indomethacin (3 μM), charybdotoxin (10 nM) and apamin (100 nM). Rings are then contracted to ˜50% KPSSₘₐₓ with U46619, and, to assess endothelial vasodilator function, relaxed with increasing concentrations of acetylcholine. To confirm that differences in ACh responses are due to alterations in NO bioavailability and not changes in receptor density/function, rings are re-contracted and relaxed a second time with the Ca²⁺ ionophore, A23187. Finally, rings are relaxed with the endothelium-independent relaxing agent, isoprenaline.

Statistical analysis: In rabbits, endpoint measures in collared arteries (drug- and vehicle-treated) is expressed relative to the same measures in the proximal non-collared section of the same artery. The effects of a particular drug versus vehicle on these measures will be compared within animal via Student's Paired t-test. To compare efficacies of different drugs and concentrations analyses are performed across animals via Tukey Kramer's test after one-way ANOVA. Likewise, in the ligation model, endpoint measures in the ligated artery will be expressed relative to the same measures in the contralateral sham operated artery. The effects of drugs or antisense on these measures are compared back to those in vehicle-treated animals via Tukey Kramer's test after one-way ANOVA. Finally, the effects of drugs or targeted gene deletion on endpoint measures in ApoE⁴ mice will also be compared across animals via Tukey Kramer's test after one-way ANOVA. Values of P<0.05 are considered significant.

Measurement of arterial blood pressure: Rats are briefly anaesthetized (ketamine 80 mg/kg ip plus xylazine 10 mg/kg ip) and a saline-filled cannula is inserted in a femoral artery. The cannula is connected to a pressure transducer and a chart recorder. When arterial pressure is observed to be stable (within 5 minutes) mean arterial pressure is calculated and recorded.

Angiotensin II-induced experimental hypertension: On Day 0, rats are briefly anaesthetized (ketamine 80 mg/kg ip plus xylazine 10 mg/kg ip) and an osmotic minipump containing either saline or suramin is implanted subcutaneously. The dose rate of suramin is 300 mg/kg per 14 days. On Day 7, rats are again anaesthetized and another minipump containing saline or angiotensin II is implanted subcutaneously. The dose rate of angiotensin II is 5 mg/kg per 7 days. On Day 14, each rat is again anaesthetized and a cannula was inserted into a femoral artery for measurement of blood pressure. Angiotensin II causes a large increase in mean arterial pressure (of approx. 60-80 mmHg) in control rats.

Subarachnoid hemorrhage: On Day 0, rats are briefly anaesthetized (pentobarbital 50 mg/kg ip) and an osmotic minipump containing either saline or suramin is implanted subcutaneously. The dose rate of suramin is 300 mg/kg per 7 days. On Day 5, rats are again anaesthetized and 0.3 ml of blood is withdrawn from a femoral artery and injected into the cerebrospinal fluid around the ventral surface of the brain via the cisterna magna. In some control rats, saline is injected into the cerebrospinal fluid instead of arterial blood. The rat is allowed to recover for a further 2 days, and is then again anaesthetized on Day 7 for study.

Measurement of cerebral artery responses in vivo: Rats are anaesthetized with pentbarbital (50 mg/kg ip) and anaesthesia is maintained with supplemental pentobarbital (10-20 mg/kg per h iv). The basilar artery on the ventral surface of the brainstem is surgically exposed using a cranial window approach. Basilar artery diameter is continuously measured using a computer-based image tracking device. The endothelium-dependent vasodilator acetylcholine is superfused over the basilar artery at a steady concentration of 1 micromolar for 3-5 minutes, and the increase in diameter is measured. The concentration of acetylcholine is then increased to 10, and then 100 micromolar in a similar manner and increases in diameter recorded. Finally, the maximum diameter capacity of the artery is recorded by measuring the response to the combination of 100 micromolar sodium nitroprusside plus 10 micromolar nimodipine. Responses to acetylcholine are then expressed as a percent of this maximum response. Impaired endothelial function, for example following experimental subarachnoid hemorrhage, will be confirmed by a weaker vasodilator response to acetylcholine in comparison to responses in control animals.

### EXAMPLE 6

### Inhibitory Effect of Chronic Suramin Treatment on Atherosclerotic Lesion Formation in Aortas of Fat-Fed ApoE Mutant Mice

Twelve-week-old ApoE mutant mice were fed a high fat diet for a further 4 months. During this 4 month period, the mice were given weekly subcutaneous injections of saline or suramin. Specifically, mice were initially dosed with two weekly injections of 300 mg/kg suramin. Four weeks later a 25 mg/kg dose of suramin was administered, and then weekly doses of 15 mg/kg suramin were administered for a further 11 weeks. At the end of the treatment period, mice were killed by anaesthetic overdose and the aorta was removed, cleaned of adherent fat on the adventitial surface, and then cut longitudinally along the entire length. The atherosclerotic lesions were stained with oil red 0, and vessels were fixed in formalin. The en face surface of the aorta was then imaged and lesion area (red-stained) quantified and expressed as a percent of total luminal surface area for each of: total aorta, thoracic aorta, abdominal aorta, and aortic arch. The findings indicate that aortas from mice treated with suramin contained markedly smaller lesion areas (whether considered as total aorta, thoracic aorta, or abdominal aorta) than vessels from saline-treated control mice. Consequently, chronic inhibition of Nox4-containing NADPH-oxidase by suramin inhibits atherosclerotic lesion formulation.

### EXAMPLE 7

### Protective Effect of Chronic Suramin Treatment on Acetylcholine-Induced Dilator Responses of the Rate Basilar Artery In Vivo after Subarachnoid Haemorrhage

On Day 0, rats were briefly anaesthetized and an osmotic minipump containing either saline or suramin was implanted subcutaneously. The dose rate of suramin was 300 mg/kg per 7 days. On Day 5, rats were again anaesthetized and 0.3 ml of blood was withdrawn from a femoral artery and injected into the cerebrospinal fluid around the ventral surface of the brain via the cisterna magna. In some rats, saline was injected into the cerebrospinal fluid instead of arterial blood. The rat was allowed to recover for a further 2 days, and then again anaesthetized on Day 7. The basilar artery on the ventral surface of the brainstem was then surgically exposed using a cranial window approach. Basilar artery diameter was continuously measured using a computer-based image tracking device. Acetylcholine was superfused over the basilar artery at a steady concentration of 1 micromolar for 3-5 minutes, and the increase in diameter was measured. The concentration of acetylcholine was then increased to 10, and then 100 micromolar in a similar manner and increases in diameter recorded. Finally, the maximum diameter capacity of the artery was recorded by measuring the response to the combination of 100 micromolar sodium nitroprusside plus 10 micromolar nimodipine. Responses to acetylcholine were then expressed as a percent of this maximum response. Concentration-response curves were graphed and statistically compared. It was found that, compared with responses in rats receiving saline only on Days 0 and 5, the response to acetylcholine was substantially reduced in animals pretreated with saline and subjected to subarachnoid hemorrhage. Importantly, responses to acetylcholine were not impaired after subarachnoid hemorrhage in animals pretreated with suramin. Chronic inhibition of Nox4-containing NADPH-oxidase, therefore, by suramin prevents impairment of endothelial function in cerebral arteries after subarachnoid hemorrhage.

### EXAMPLE 8

### Inhibitory Effect of Chronic Suramin Treatment on NADPH-Induced Superoxide Production by the Rat Basilar Artery In Vitro after Subarachnoid Haemorrhage

On Day 0, rats were briefly anaesthetized and an osmotic minipump containing either saline or suramin was implanted subcutaneously. The dose rate of suramin was 30 or 300 mg/kg per 7 days. On Day 5, rats were again anaesthetized and 0.3 ml of blood was withdrawn from a femoral artery and injected into the cerebrospinal fluid around the ventral surface of the brain via the cisterna magna. In some rats, saline was injected into the cerebrospinal fluid instead of arterial blood. The rat was allowed to recover for a further 2 days, and then was killed by anaesthetic overdose on Day 7.

The brain was removed and the basilar artery was isolated and then incubated with 5 micromolar lucigenin, 100 micromolar NADPH and 3 millimolar diethyldithiocarbamate. Superoxide production was measured using lucigenin-enhanced chemiluminescence. It was found that in rats that had been implanted with saline-containing minipumps, injection of blood into the cerebrospinal fluid resulted in higher superoxide production from the basilar artery. In contrast, superoxide production by basilar arteries from rats pretreated with either dose of suramin was not different from levels measured in non-operated control rats.

Accordingly, chronic inhibition of Nox4-containing NADPH-oxidase by suramin prevents excessive superoxide production by cerebral arteries after subarachnoid hemorrhage.

### EXAMPLE 9

### Inhibitory Effect of Chronic Suramin Treatment on Hypertension Caused by Infusion of Angiotensin II

On Day 0, rats were briefly anaesthetized and an osmotic minipump containing either saline or suramin was implanted subcutaneously. The dose rate of suramin was 300 mg/kg per 14 days. On Day 7, rats were again anaesthetized and another minipump containing saline or angiotensin II was implanted subcutaneously. The dose rate of angiotensin II was 5 mg/kg per 7 days. On Day 14, each rat was again anaesthetized and a cannula was inserted into a femoral artery for measurement of blood pressure. Angiotensin II caused a large increase in blood pressure in rats pretreated with saline. In contrast, the increase in blood pressure by angiotensin II was prevented by approximately 60% in rats pretreated with suramin.

Accordingly, chronic inhibition of Nox4-containing NADPH-oxidase by suramin inhibits hypertension caused by angiotensin II.

### EXAMPLE 10

### Prediction of Transmembrane Regions and Topology of Exramembrane Regions

This example utilizes PSORT software (http://psort.nibb.ac.jp/) to predict both the transmembrane domains and the topology of mouse Nox4 based on its amino acid sequence (genebank accession no. NP_{—}056575). This example shows that the NADPH binding site of Nox4 is extracellularly located.

Protein Sorting Signals and Localisation Sites (PSORT) software program was used to predict the topology of the C-terminal tail of both gp91phox and Nox4 which contains the NADPH binding cleft (Lambeth et al., Trends Biochem Sci., 25:459-61, 2000). Amino acid sequences of mouse homologues of the NADPH oxidase subunits gp91phox (genebank accession no. AAB05997) and Nox4 (genebank accession no. NP_{—}056575) were obtained from the NCBI website, (http://www.ncbi.nlm.nih.gov/entrez/).

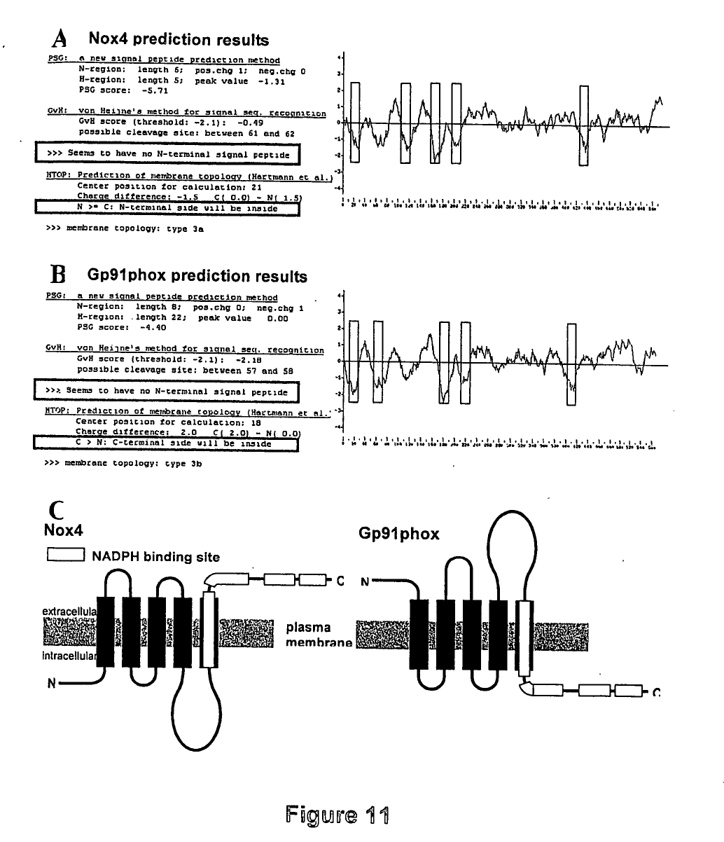

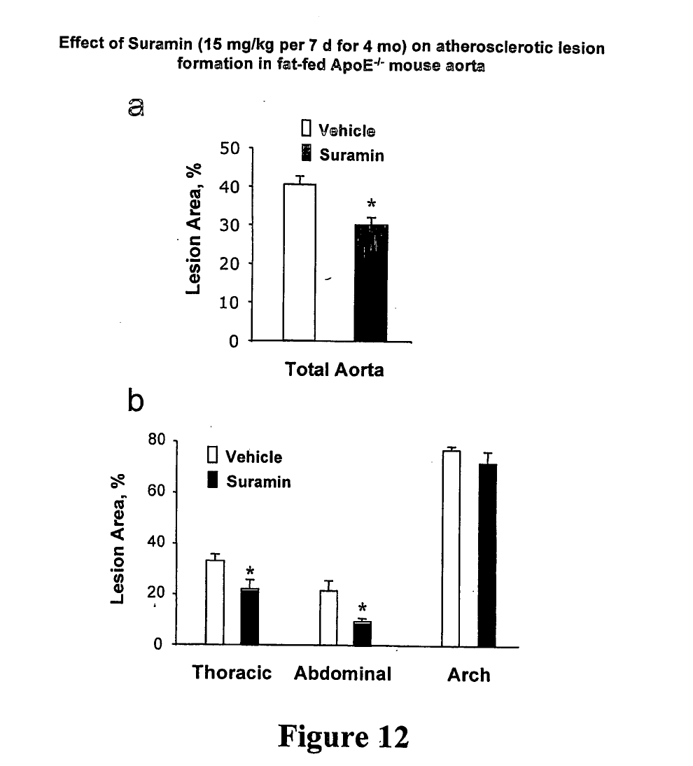

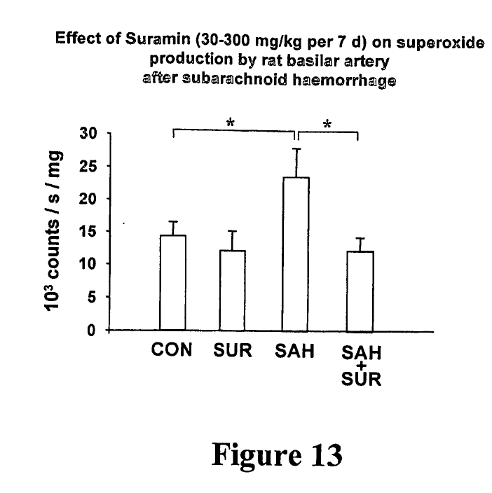

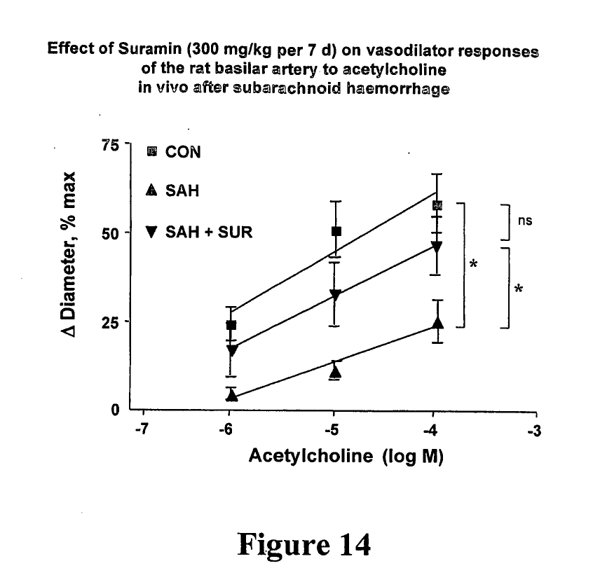

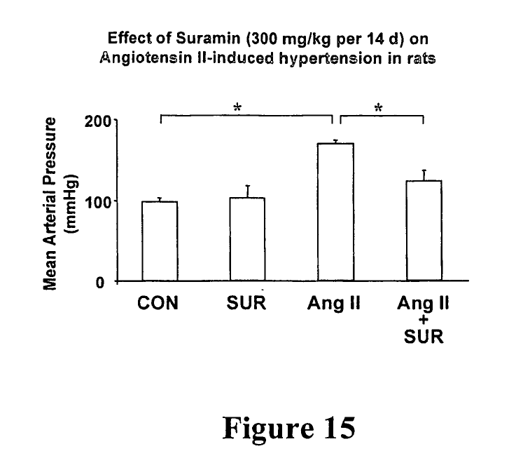

Nox4 was found to have 5 hydrophobic regions that are predicted to be transmembrane domains (Table 8; FIG. 11A). PSORT was also unable to detect any N-terminal signal peptide on Nox4. Moreover, based on the “positive inside rule” which states the more positive end (N or C-terminal) of the first transmembrane domain almost always resides on the cytosolic side (Hartman et al., Proc. Natl. Acad. Sci, USA, 86:5786-90, 1989), the N-terminus of Nox4 is predicted to be intracellular while the C-terminal tail hangs extracellularly (FIG. 11A). The NADPH binding site of Nox4 is predicted to reside at amino acid positions 425-442, 459-468, 515-534 and 541-552 (Lambeth supra). This places the majority of the NADPH binding site on the C-terminal tail beyond the 5^{th} transmembrane domain, thus suggesting that it is located extracellularly (FIG. 11C). For comparison, we also analysed the membrane topology of mouse gp91phox which was predicted to also have S transmembrane domains (Table 8; FIG. 11B). However, unlike Nox4, the C-terminal tail of gp91phox, which contains the NADPH binding site (amino acids positions 403-420, 441-450, 505-514, and 531-542¹), is predicted to be intracellular (FIG. 11C).

The experiments outlined in this example and the other examples provide evidence that the NADPH binding site on Nox4 is located on the extracellular side of the plasma membrane.

The topology predictions using PSORT software indicates that the NADPH binding site of Nox4 is located extracellularly. PSORT predicted that Nox4 contains an uncleavable signal anchor sequence which represents the 1^{st} transmembrane domain. Based on the “positive inside rule” (Hartmann supra), the N-terminal side of the 1^{st} transmembrane which is mole positively charged than its C-terminal side is predicted to be inside the cell. Given that PSORT also predicted Nox4 to have a total of 5 transmembrane spanning regions, the C-terminus containing the NADPH binding site would then be located extracellularly. In contrast, gp91phox, whose C-terminal side of the 1^{st} transmembrane domain is more positively charged than the N-terminal side, is predicted to have its N-terminus on the outside of the cell. Thus, with 5 transmembrane spanning regions predicted, the C-terminus of gp91phox containing the NADPH binding site should be intracellular. An intracellular NADPH binding site for gp91phox provides an explanation for why suramin and reactive blue-2 were ineffective at inhibiting NADPH oxidase in intact J774 mouse macrophages.

TABLE 8
Predictions of transmembrane domains on Nox4 and
gp91phox catalytic subunits by PSORT.

| Transmembrane | 1. Amino Acid Positions | |
|---|---|---|
| domain | Nox4 | Gp91phox |
| 1 | 14-30 | 11-27 |
| 2 | 106-122 | 56-72 |
| 3 | 159-175 | 174-190 |
| 4 | 196-212 | 212-228 |
| 5 | 425-441 | 403-419 |

### EXAMPLE 11

### Demonstration that Suramin does not Penetrate the Plasma Membrane of Mouse Vascular Smooth Muscle Cells

Mouse vascular smooth muscle cells (VSMCs), previously grown to confluence in 60 mm diameter culture dishes in Dulbecco's Modified Eagles Medium (DMEM) supplemented with 10% v/v foetal bovine serum are trypsinized and plated in a 1:4 ratio into 60 mm culture dishes containing Thermanox (Reg. Trademark) (Nunc, II, USA) or gelatin-coated glass tissue culture cover slips. VSMCs are allowed to grow for 3 days (i.e. until they are approximately 50% confluent). Cells are washed once with Krebs-Hepes buffer to remove all traces of phenol red (present in DMEM), and then incubated for ˜2 hours in the same buffer at 37° C. Next, VSMCs are treated with suramin (100 μM) for 45 minutes, as at this concentration and duration of incubation it is sufficient to abolish NADPH-driven superoxide production in VSMCs (eg. see FIG. 2A). The coverslip-containing cells are then removed from the culture dish and placed on a microscope slide in an inverted position. To keep the cells moist, a 20 μl droplet of suramin (100 μM)-containing Krebs-Hepes are added to the slide prior to coverslipping. The slide is then placed on the stage of either a confocal microscope coupled to a UV laser, or a fluorescent microscope coupled to a mercury lamp. The intrinsic fluorescent properties of suramin are used to visualize its compartmentalization in the VSMC preparation. Suramin are excited with light of wavelength 315 or 330 nm and emission measured in the range of 350-450 nm (Fleck et al., J. Biol. Chem. 278: 47670-77, 2000). By imaging a planar-(Z-) section through the VSMCs, intense fluorescence will be demonstrated around the plasma membrane indicating that suramin is bound to an extracellular binding site(s), presumably Nox4. A ‘dull glow’ around each cell (i.e. suramin in the bathing solution) is indicative of extracellular location. By contrast the intracellular compartment of the cells devoid of both ‘the dull glow’ and any spots of intense fluorescence indicates that suramin does not achieve sufficient intracellular penetration to significantly interact with any intracellular binding sites.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Ago et al., Circulation 109(2):227-233, 2004
Altschul et al., Nucl. Acids Res. 25:3389-3402, 1997;
Arthur et al., Arteriosclerosis, Thrombosis and Vascular Biology 17: 737-740, 1997;
Arthur et al., J. Vasc. Res. 31: 187-194, 1994;
Ausubel et al., “Current Protocols in Molecular Biology” John Wiley & Sons Inc, 1994-1998, Chapter 15;
Babior, Blood 93: 1464-1476, 1999;
Bandyopadhyay and Temin, Mol. Cell. Biol. 4: 749-754, 1984;
Barry-Lane et al., J. Clin. Invest. 108: 1513-1522, 2001;
Belkhiri et al., Lab. Invest. 77: 533-539, 1997;
Bengsston et al., In: Australian Health and Medical Research Congress, Melbourne, Australia, p 1203, 2002;
Bennett et al., J. Clin. Invest. 93:820-828, 1994;
Berglund et al., Biotechnology 11: 916-920, 1993;
Berkner et al., BioTechniques 6; 616-629, 1988;
Berkner, Curr. Top. Microbiol. Immunol. 158: 39-66, 1992;
Beswick et al., Hypertension 37: 781-786, 2001;
Beswick et al., Hypertension 38: 1107-1111, 2001;
Bonner and Laskey, Eur. J. Biochem. 46: 83, 1974;
Breakefield and Geller, Mol. Neurobiol. 1: 339-371, 1987;
Breslow, Science 272: 685-688, 1996;
Buchschacher and Panganiban, J. Virol. 66: 2731-2739, 1982;
Cai and Harrison, Circ. Res. 87: 840-844, 2000;
Chothia et al., J. Mol. Biol. 196:901, 1987
Coligan et al., Current Protocols in Immunology, 1991-1997
Cuzzocrea et al., Shock 14: 150-160, 2000;
De Meyer et al., J. Cardiovasc. Pharmacol. 29(12): S205-207, 1992;
Drummond et al., Br. J. Pharmacol. 129: 811-819, 2000;
Drummond et al., Circulation 104: II-71, 2001;
Drummond, In: Australian Health and Medical Research Congress, Melbourne, Australia, pp 335, 2002;
Dusting et al., American Journal of Cardiology 76: 24E-27E, 1995;
Dusting et al., Clinical and Experimental Pharmacology and Physiology 25: S34-41, 1998;
Dusting et al., J. Cardiovasc. Pharmacol. 16: 667-674, 1990;
Erickson et al., Science 249: 527-533, 1990;
European Patent Publication No. 0 239 400
Fink et al., Ann. Rev. Neurosci. 19: 265-287, 1996;
Fink et al., Hum. Gene Ther. 3: 11-19, 1992;
Fleck et al., J. Biol. Chem. 278: 47670-77, 2000
Freese et al., Biochem. Pharmacol. 40: 2189-2199, 1990;
Gaspari et al., Clinical and Experimental Pharmacology and Physiology 27: 653-655, 2000b;
Gaspari et al., In: The Biology of Nitric Oxide, Part 7, Ed. S. Moncada, L. Gustafson, P. Wiklund and E. A. Higgs, Portland Press, London, pp. 72-73, 2000a;
Gefter et al., Somatic Cell Genet. 3:231-236, 1977
Gorziglia and Kapikian, J. Virol. 66: 4407-4412, 1992;
Griendling et al., Cir. Res. 86: 494-501, 2000;
Griendling and Ushio-Fukai, J. Lab. Clin. Med. 132: 9-15, 1998;
Gryglewski et al., Nature 320: 454-456, 1986;
Guzik et al., Cir. Res. 86: E85-90, 2000;
Hamilton et al., Hypertension 40: 755-762, 2002;
Hartman et al., Proc. Natl. Acad. Sci, USA, 86:5786-90, 1989
Helseth et al., J. Virol. 64: 2416-2420, 1990;
Herbert et al., J. Cell Physiol. 170: 106-114, 1997;
Hodgson, Bio/Technology 9: 19-21, 1991;
Holzmann et al., Biomedical Mass Spectrophotometry 12: 659-663, 1985;
Jentsch et al., J. Gen. Virol. 68: 2183-2192, 1987;
Jiang et al., European Journal of Pharmacology 424: 141-149, 2001;
Johnson et al., “Peptide Turn Mimetics” in Biotechnology and Pharmacy, Pezzuto et al., Eds., Chapman and Hall, New York, 1993;
Johnson et al., J. Virol. 66: 2952-2965, 1992;
Jones et al., Nature 321:522-525, 1986
Kockx et al., Arterioscler. Thromb. 12: 1447-1457, 1992;
Kockx et al., Arterioscler. Thromb. 13: 1874-1884, 1993;
Kohler et al., Nature 256: 495-499, 1975
Kohler et al., Eur. J. Immunol. 6(7):511-519, 1976
Kozbor et al., Methods in Enzymology 121:140, 1986
Kumar and Linder, Arterioscler. Thromb. Vasc. Biol. 17: 2238-2244, 1997;
Kyte and Doolittle, J. Mol. Biol. 157: 105-132, 1982;
Lambeth et al., Trends Biochem. Sci. 25: 459-461, 2000;
Lassegue et al., Circ. Res. 88: 888-894, 2001;
Laursen et al., Circulation 103: 1282-1288, 2001;
Liu et al., Proc. Natl. Acad. Sci. USA 84:3439-3443, 1987
Lo et al., Am. J. Physiol. 264: L406-412, 1993;
Lynch and Frei, J. Lipid Res. 34: 1745-1753, 1993;
Madzak et al., J. Gen. Virol. 73: 1533-1536, 1992;
Mann and Baltimore, J. Virol. 54: 401-407, 1985;
Margolskee, Curr. Top., Microbiol. Immunol. 158: 67-95, 1992;
Marmur and Doty, J. Mol. Biol. 5: 109, 1962;
McPherson et al., Arterioscler. Thromb. Vasc. Biol. 21: 791-796, 2001;
Merrilees et al., J. Vasc. Res. 31: 322-329, 1994;
Miller et al., J. Virol. 62: 4337-4345, 1988;
Miller et al., Mol. Cell. Biol. 5: 431-437, 1985;
Miller, Curr. Top. Microbiol. Immunol. 158: 1-24, 1992;
Morgan et al., (U.S. Pat. No. 6,180,377)
Moss, Curr. Top. Microbiol. Immunol. 158: 25-38, 1992;
Moss, Proc. Natl. Acad. Sci. USA 93: 11341-11348, 1996;
Munder et al., Appl. Microbiol. Biotechnol. 52(3): 311-320, 1999;
Muzyczka, Curr. Top. Microbiol. Immunol. 158: 97-129, 1992;
Naldini et al., Science 272: 263-267, 1996;
Nassar et al., Eur. J. Pharmacol. 436: 111-118, 2002;
Nickel et al., Arzneimittel-Forschung 36: 1153-57, 1986;
Ohi et al., Gene 89: 279-282, 1990;
Page et al., J. Virol. 64: 5270-5276, 1990;
Paravicini et al., Circulation Research 91: 54-61, 2002;
Petropoulos et al., J. Viol. 66: 3391-3397, 1992;
Quantin et al., Proc. Natl. Acad. Sci. USA 89: 2581-2584, 1992;
Queen et al. (U.S. Pat. No. 6,180,377)
Remington's Pharmaceutical Sciences, 18^{th} Ed., Mack Publishing, Company, Easton, Pa., U.S.A., 1990;
Richmann et al., Nature 332:323-327, 1988
Ritchie et al., European Journal of Pharmacology 461: 171-179, 2003;
Rosenfeld et al., Cell 68: 143-155, 1992;
Russell and Hirata, Nature Genetics 18: 323-328, 1998;
Schnackenberg et al., Hypertension 33: 424-428, 1999;
Schneider et al., Nature Genetics 18: 180-183, 1998;
Shi et al., Arterioscler Thromb. Vasc. Biol. 21: 739-745, 2001;
Shimada et al., J. Clin. Invest. 88:1043-1047, 1991;
Shulman et al., Nature 276:269-270, 1978
Simons et al., Nature 359: 67-70, 1992;
Skatchkov et al., Biochem. Biophys. Res. Commun. 254: 319-324, 1999;
Sorge et al., Mol. Cell. Biol. 4: 1730-1737, 1984;
Souza et al., Am. J. Physiol. Heart Circ. Physiol. 280: H658-667, 2001;
Stolk et al., Am. J: Respir. Cell Mol. Biol. 11: 95-102, 1994;
Stratford-Perricaudet et al., Hum. Gene Ther. 1: 241-256, 1990;
Stuehr et al., Faseb J. 5: 98-103, 1991;
Summerton and Weller, Antisense and Nucleic Acid Drug Development 7: 187-195, 1997;
Sun et al., European Journal of Pharmacology 320: 29-35, 1997;
Tarpey and Fridovich, Cir. Res. 89: 224-236, 2001;
Toyama et al., Monoclonal Antibody, Experiment Manual, published by Kodansha Scientific, 1987
Trowbridge, J. Exp. Med. 148(1):220-227, 1982
Ushio-Fukai et al., J. Biol. Chem. 271: 23317-23321, 1996;
Verhoeyen et al., Science 239:1534-1536, 1988
Villa et al., Circ. Res. 76: 505-513, 1995;
Volk et al., J. Virol. 42(1):220-227, 1982
Wang et al., Br. J: Pharmacol. 110: 1232-1238, 1993;
Wells, Methods Enzymol. 202: 2699-2705, 1991;
West et al., Arterioscler Thromb. Vasc. Biol. 21: 189-194, 2001;
Wilkinson et al., Nucleic Acids Res. 20: 2233-2239, 1992;
Winer et al., Anal. Biochem. 270: 41-49, 1999;
Yin and Dusting, Clinical and Experimental Pharmacology and Physilogy 24: 436-438, 1997;
Yin et al., Journal of Vascular Research 35: 156-164, 1998;
Young et al., Nat. Biotechnol. 16(10): 946-950, 1998;
Yusuf et al., N. Engl. J. Med. 342: 154-160, 2000.

1. An isolated cell-impermeable compound which inhibits an NADPH oxidase activity in a particular cell wherein said NADPH oxidase comprises an extracellular NADPH binding domain.

2. The compound of claim 1 wherein the compound inhibits or reduces the generation of superoxide, hypochlorite, lipid peroxides, peroxynitrite, hydrogen peroxide and/or hydroxyl radicals.

3. The compound of claim 1 wherein the compound interacts or binds to an extracellularly exposed NADPH-binding β-subunit of the NADPH oxidase.

4. The compound of claim 3 wherein the NADPH-binding β-subunit is Nox4.

5. The compound of claim 4 wherein all or a portion of the Nox4 is present on the outside of the cell membrane.

6. The compound of claim 1 wherein the cell is selected from a cell of the smooth muscle-containing vasculature, endothelial cell-containing vasculature, adventitial fibroblast-containing vasculature and a non-vasculature system.

7. The compound of claim 4 wherein the compound is a benzamide or a derivative or analog thereof.

8. The compound of claim 4 wherein the compound is an aryl sulphonate or a derivative or analog thereof.

9. The compound of claim 8 wherein the aryl sulphonate or derivative or analog is suramin or a derivative or analog thereof.

10. The compound of claim 8 wherein the derivative of suramin is selected from a derivative disclosed in FIG. 1.

11. The compound of claim 4 wherein the compound is diphenyleneiodonium (DPI) or 4-hydroxy-2,2,6,6-tetramethyl piperidinixyl (tempol) or a derviative or analogue or homolog.

12. The compound of claim 4 wherein the compound is apocynin, Reactive blue-2 or PPADS or analogs or derivatives thereof.

13. The compound of claim 4 wherein the compound is a superoxide scavenger.

14. The compound of claim 1 wherein the compound binds to the amino acid sequence set forth in SEQ ID NO:2 or SEQ ID NO:4 or SEQ ID NO:6 or SEQ ID NO:8 or an amino acid sequence having at least about 60% identity to SEQ ID NO:2 or SEQ ID NO:4 or SEQ ID NO:6 or SEQ ID NO:8.

15. The compound of claim 1 wherein the compound is a peptide, polypeptide or protein, non-proteinaceous chemical molecule or synthetic molecule.

16. The compound of claim 14 wherein the compound binds to a nucleic acid molecule comprising a nucleotide sequence set forth in SEQ ID NO:1 or SEQ ID NO:3 or SEQ ID NO:5 or SEQ ID NO:7 or a nucleotide sequence having at least about 60% identity thereto or its complementary form or a nucleotide sequence capable of hybridizing to SEQ ID NO:1 or SEQ ID NO:3 or SEQ ID NO:5 or SEQ ID NO:7 or a complementary form thereof under low stringency conditions.

17. The compound of claim 16 wherein the compound is a nucleotide antisense molecule or a chemically modified form or analog thereof.

18. The compound of claim 17 wherein the compound is a nucleotide sense molecule or a chemically modified form or analog thereof.

19. A composition comprising a compound of claims 1 and one or more pharmaceutically acceptable carriers, diluents and/or excipients.

20. A method for the treatment or prophylaxis of a condition or event in a mammal, said method comprising administering to said mammal an effective amount of a compound of claim 1.

21. The method of claim 20 wherein the mammal is a human.

22. The method of claim 20 wherein the mammal is a laboratory test animal.

23. The method of claim 20 wherein the condition or event includes pathologies such as atherosclerosis and arteriosclerosis, cadiovascular complications of Type I and II diabetes, intimal hyperplasia, coronary heart disease, cerebral, coronary or arterial vasospasm, endothelial dysfunction, heart failure including congestive heart failure, sepsis, peripheral artery disease, restenosis and restenosis after angioplasty, stroke, vascular complications after organ transplantation, cardiovascular complications arising from viral and bacterial infections as well as any conditions which may be independent or secondary to another condition including mycardial infarction, hypertension, formation of atherosclerotic plaques, platelet aggregations, angina, aneurysm, transient ischemic attack, abnormal oxygen flow and/or delivery, atrophy or organ damage, pulmonary embolus, thrombotic or a generalized arterial or venous condition including endothelial dysfunction, a thrombotic event including deep vein thrombosis or damage to vessels of the circulatory system or stent failure or trauma caused by a stent, pacemaker or other prosthetic device as well as reperfusion injury including any injury caused after ischemia by restoration of blood flow and oxygen delivery, gangrene, (cancer and/or abnormal tumor), stem or progenitor cell proliferation, respiratory disease (eg. asthma, bronchitis, allergic rhinits and adult respiratory distress syndrome), skin disease (psoriasis, eczema and dermatitis), and various disorders of bone metabolisms (oestoporosis, hyperparathyroidism, oestosclorosis, oestoporasis and periodontits) and renal failure.

24. The method of claim 23 wherein the cancer is selected from ABL1 protooncogene, AIDS Related Cancers, Acoustic Neuroma, Acute Lymphocytic Leukaemia, Acute Myeloid Leukaemia, Adenocystic carcinoma, Adrenocortical Cancer, Agnogenic myeloid metaplasia, Alopecia, Alveolar soft-part sarcoma, Anal cancer, Angiosarcoma, Aplastic Anaemia, Astrocytoma, Ataxia-telangiectasia, Basal Cell Carcinoma (Skin), Bladder Cancer, Bone Cancers, Bowel cancer, Brain Stem Glioma, Brain and CNS Tumors, Breast Cancer, CNS tumors, Carcinoid Tumors, Cervical Cancer, Childhood Brain Tumors, Childhood Cancer, Childhood Leukaemia, Childhood Soft Tissue Sarcoma, Chondrosarcoma, Choriocarcinoma, Chronic Lymphocytic Leukaemia, Chronic Myeloid Leukaemia, Colorectal Cancers, Cutaneous T-Cell Lymphoma, Dermatofibrosarcoma-protuberans, Desmoplastic-Small-Round-Cell-Tumour, Ductal Carcinoma, Endocrine Cancers, Endometrial Cancer, Ependymoma, Esophageal Cancer, Ewing's Sarcoma, Extra-Hepatic Bile Duct Cancer, Eye Cancer, Eye: Melanoma, Retinoblastoma, Fallopian Tube cancer, Fanconi Anaemia, Fibrosarcoma, Gall Bladder Cancer, Gastric Cancer, Gastrointestinal Cancers, Gastrointestinal-Carcinoid-Tumour, Genitourinary Cancers, Germ Cell Tumors, Gestational-Trophoblastic-Disease, Glioma, Gynaecological Cancers, Haematological Malignancies, Hairy Cell Leukaemia, Head and Neck Cancer, Hepatocellular Cancer, Hereditary Breast Cancer, Histiocytosis, Hodgkin's Disease, Human Papillomavirus, Hydatidiform mole, Hypercalcemia, Hypopharynx Cancer, IntraOcular Melanoma, Islet cell cancer, Kaposi's sarcoma, Kidney Cancer, Langerhan's-Cell-Histiocytosis, Laryngeal Cancer, Leiomyosarcoma, Leukaemia, Li-Fraumeni Syndrome, Lip Cancer, Liposarcoma, Liver Cancer, Lung Cancer, Lymphedema, Lymphoma, Hodgkin's Lymphoma, Non-Hodgkin's Lymphoma, Male Breast Cancer, Malignant-Rhabdoid-Tumour-of-Kidney, Medulloblastoma, Melanoma, Merkel Cell Cancer, Mesothelioma, Metastatic Cancer, Mouth Cancer, Multiple Endocrine Neoplasia, Mycosis Fungoides, Myelodysplastic Syndromes, Myeloma, Myeloproliferative Disorders, Nasal Cancer, Nasopharyngeal Cancer, Nephroblastoma, Neuroblastoma, Neurofibromatosis, Nijmegen Breakage Syndrome, Non-Melanoma Skin Cancer, Non-Small-Cell-Lung-Cancer-(NSCLC), Ocular Cancers, Oesophageal Cancer, Oral cavity Cancer, Oropharynx Cancer, Osteosarcoma, Ostomy Ovarian Cancer, Pancreas Cancer, Paranasal Cancer, Parathyroid Cancer, Parotid Gland Cancer, Penile Cancer, Peripheral-Neuroectodermal-Tumors, Pituitary Cancer, Polycythemia vera, Prostate Cancer, Rare-cancers-and-associated-disorders, Renal Cell Carcinoma, Retinoblastoma, Rhabdomyosarcoma, Rothmund-Thomson Syndrome, Salivary Gland Cancer, Sarcoma, Schwannoma, Sezary syndrome, Skin Cancer, Small Cell Lung Cancer (SCLC), Small Intestine Cancer, Soft Tissue Sarcoma, Spinal Cord Tumors, Squamous-Cell-Carcinoma-(skin), Stomach Cancer, Synovial sarcoma, Testicular Cancer, Thymus Cancer, Thyroid Cancer, Transitional-Cell-Cancer-(bladder), Transitional-Cell-Cancer-(renal-pelvis-/-ureter), Trophoblastic Cancer, Urethral Cancer, Urinary System Cancer, Uroplakins, Uterine sarcoma, Uterus Cancer, Vaginal Cancer, Vulva Cancer, Waldenstrom's-Macroglobulinemia and Wilms' Tumour.

25. The method of claim 23 wherein the condition or event of the systemic vasculature is atherosclerosis or endothelial dysfunction.

26. The method of claim 20 wherein the amount of compound administered is effective to inhibit or reduce formation of superoxide and/or downstream ROS from VSMCs and/or endothelial cell-containing vasculature and/or adventital fibroblast-containing vasculature and/or non-vascular systems

27. The method of claim 20 wherein the amount of compound administered is effective to inhibit or reduced formation of superoxide, hypochlorite, lipid peroxides, peroxynitrite, hydrogen peroxide and/or hydroxyl radicals in or by VSMCs and/or endothelial cell-containing vasculature and/or adventitial fibroblast-containing vasculature and/or non-vascular system.

28. The method of claim 20 wherein the compound is a nucleic acid molecule or a chemically modified or analog form thereof.

29. The method of claim 20 wherein the compound is suramin or a derivative or analog thereof.

30. The method of claim 20 wherein the compound is tempol or DPI or a derivative or analog thereof.

31. The method of claim 20 wherein the compound is Reactive blue-2 or PPADS or a derivative or analog thereof.

32. Use of a benzamide and/or aryl sulphonate and derivative or analog in the manufacture of a medicament for the treatment or prophylaxis of a condition or event in a mammalian or non-mammalian animal.

33. Use of a Nox4 inhibitor in the manufacture of a medicament for the treatment or prophylaxis of a condition or event in a mammalian or non-mammalian animal wherein all or a portion of the Nox4 is extracellularly exposed.

34. Use of suramin or a derivative or analog thereof in the manufacture of a medicament for the treatment or prophylaxis of a condition or event in a mammalian or non-mammalian animal.

35. Use of tempol in the manufacture of a medicament for the treatment or prophylaxis of a condition or event in a mammalian or non-mammalian animal.

36. Use of DPI in the manufacture of a medicament for the treatment or prophylaxis of a condition or event in a mammalian or non-mammalian animal.

37. A non-human animal model comprising a mutation in or flanking a genetic locus encoding Nox4.

38. The animal model of claim 37 wherein the mutation is an insertion, deletion, substitution or addition to the Nox4 coding sequence or its 5′ or 3′ untranslated region.

39. The animal model of claim 37 wherein the mutation is a loxP insertion flanking the Nox4 gene.

40. A multi-part pharmaceutical pack comprising a first part comprising a compound of claim 1 or at least a second or more parts comprising one or more therapeutic agents useful in ameliorating the symptoms or effects of a condition or event in a mammalian or non-mammalian animal.

41. The multi-part pharmaceutical pack of claim 40 further comprising a part comprising an agonist of Nox4-inhibitor interaction.
